(19) United States
(12) Patent Application Publication
(10) Pub. No.: US 2011/0269779 A1
(43) Pub. Date: Nov. 3, 2011

(54) METHODS AND COMPOSITIONS FOR TREATMENT OF OPHTHALMIC CONDITIONS

(75) Inventors: Troy Edward Wilson, San Marino, CA (US); Christian Rommel, La Jolla, CA (US); Yi Liu, San Diego, CA (US); Pingda Ren, San Diego, CA (US)

(73) Assignee: INTELLIKINE, INC., La Jolla, CA (US)

(21) Appl. No.: 13/127,973

(22) PCT Filed: Nov. 17, 2009
(86) PCT No.: PCT/US2009/064717

§ 371 Date: Jul. 12, 2011

Related U.S. Application Data
(60) Provisional application No. 61/115,930, filed on Nov. 18, 2008.

(51) Int. Cl. A61K 31/519; A61K 31/436; A61P 27/02; C12N 5/02

(52) U.S. Cl. 514/262.1; 435/375; 514/291; 514/264.1

(57) ABSTRACT

The present invention provides chemical entities or compounds and pharmaceutical compositions thereof that are capable of modulating signal transduction by certain protein kinases such as mTor, tyrosine kinases, and/or lipid kinases such as PB kinase in an ocular tissue. Also provided in the present invention are methods of using these compositions to modulate activities of one or more of these kinases, especially for therapeutic applications.

### BACKGROUND OF THE INVENTION

Aberrant neovascularization or vascular permeability in the eye is a major cause of many ocular disorders including age-related macular degeneration (AMD), retinopathy of prematurity (ROP), ischemic retinal vein occlusion, diabetic retinopathy (DR), and neovascular glaucoma (NVG). AMD and DR are amongst the most common causes of severe, irreversible vision loss in humans. In these angiogenesis-related ocular diseases, central vision loss is secondary to angiogenesis, a process by which new blood vessels from pre-existing vasculature are developed and/or vascular permeability properties are altered.

Inflammation is another cause of many eye diseases. In the case of dry-type age-related macular degeneration, activation of the complement cascade can lead to death of photoreceptor and retinal pigment epithelial cells and eventually loss of the visual field. Another cause of ocular disorders is an elevated intraocular pressure, which can lead to diseases such as glaucoma.

The angiogenic process generally involves the activation of quiescent endothelial cells in pre-existing blood vessels. The normal retinal vasculature is resistant to neovascular stimuli, and little endothelial cell proliferation typically takes place there. Neovascular stimuli include tissue hypoxia, inflammatory cell infiltration and penetration barrier breakdown which can increase cytokine production, expression of cell adhesion molecules such as integrins, and upregulation of proteinase activity. These stimuli in turn result in angiogenesis, which can disrupt the organizational structure of the neural retina, damage the integrity of the inner limiting membranes surrounding the vitreous, and disrupt endothelial tight junctions leading to increased vascular leakage and retinal edema.

One potential mechanism of an improper inflammatory process in the eye involves activation of T cells, which activates the complement cascade and produces reactive oxygen species, which facilitate lipid peroxidation, which in turn, is toxic to neuronal cells such as photoreceptor and retinal pigment epithelial cells.

Currently, there is no cure for these ocular neovascularization diseases. Current treatment procedures for AMD include laser photocoagulation and photodynamic therapy (PDT). The effects of photocoagulation on AMD are achieved through the thermal destruction of retinal cells. PDT usually requires slow infusion of a specific wavelength absorbing dye, followed by application of non-thermal laser light that is absorbed by the dye which leads to localized destruction or perturbation of cells that have absorbed a significant fraction of the dye. PDT treatment is often repeated several times a year and is associated with side effects including headaches, blurring, decreased visual acuity, and gaps in vision in approximately 4% of patients. Subjects receiving PDT must also avoid direct sunlight for up to 5 days to avoid sunburn. Refinements of PDT include the use of verteporfin or other drugs in combination with PDT. Newer treatments include VEGF and VEGFR antagonists such as monoclonal antibodies (ranibizumab) or antibody fragments (bevacizumab) and nucleic acid aptamers (pegaptanib) that bind to and inhibit the interaction between VEGF and its receptor.

There are also few treatments for reducing either the local inflammatory response or reducing the intraocular pressure in the eye. Currently, one of the only treatments for glaucoma is Xalatan, a prostaglandin FP2 agonist, which facilitates drainage of the intraocular fluid through the uveoscleral route. However, uveoscleral outflow thought to represent only 20% of the total outflow, with the remaining 80% draining through the trabecular meshwork.

Thus, patients who have been diagnosed with an ocular neovascularization disease such as AMD continue to experience deterioration of visual acuity over time. New methods of treatment that can either reduce neovascularization, lessen the local inflammatory response or lower the intraocular pressure to slow, decrease, or prevent deterioration of visual acuity are needed.

### SUMMARY OF THE INVENTION

Signaling proteins involved in these pathological processes include vascular endothelial growth factor (VEGF), PDGF, FGF, TNF, and IGF. In one embodiment, the present invention provides methods and compounds for modulating the vascular endothelial growth factor receptor (VEGF-R) signaling pathway in an eye, or cells derived from an eye. In some embodiment, an agent that can reduce T cell activation, thereby curbing the local inflammatory response, is provided for treatment of dry-type AMD. In some cases, the methods includes administering to a subject one or more antagonists of a PI3K or pharmaceutically acceptable salt thereof. In some cases, the one or more antagonists of PI3K inhibit one or more of PI3Kα, PI3Kδ, PI3Kγ, or PI3Kβ with an IC50 less than about 1 μM, 750 nM, 500 nM, 250 nM, 150 nM, 100 nM, 50 nM, 25 nM, 10 nM or about 1 nM. In other cases, the one or more antagonists of PI3K inhibit two or three or more of PI3Kα, PI3Kδ, PI3Kγ, or PI3Kβ with an IC50 less than about 1μM, 750 nM, 500 nM, 250 nM, 150 nM, 100 nM, 50 nM, 25 nM, 10 nM or about 1 nM.

In another embodiment, the present invention provides a method for ameliorating, treating, or mitigating an ophthalmic disease by administering one or more antagonists of mTOR and a PI3K antagonists or pharmaceutically acceptable salt thereof to the eye. In some cases, mTOR antagonist inhibits both mTORC1 and mTORC2. In some cases, the mTOR antagonist and the PI3K antagonist inhibit their target kinases with an IC50 of less than about 1 μM, 750 nM, 500 nM, 250 nM, 150 nM, 100 nM, 50 nM, 25 nM, 10 nM or about 1 nM.

In another embodiment, the present invention provides methods and compositions for inhibiting white blood cells by administering one or more pyrazolopyrimidine compounds or pharmaceutically acceptable salts thereof to the eye. In some cases, the pyrazolopyrimidine compound inhibits white blood cell proliferation (e.g. B-cells, T-cells, macrophages, neutrophils, or microlia) with an IC50 less than about 1 μM, 750 nM, 500 nM, 250 nM, 150 nM, 100 nM, 50 nM, 25 nM, 10 nM or about 1 nM.

In another embodiment, the present invention provides methods and compositions for facilitating drainage of the intraocular fluid through the trabecular meshwork, thereby lowering the intraocular pressure and alleviating the symptoms of glaucoma.

In another embodiment of the present invention methods are provided for treating ophthalmic disease by administering one or more of a pyrazolopyrimidine PI3K or mTOR antagonists to the eye of a subject.

In any one of the embodiments provided herein methods are further provided for administering the compounds of the present invention via eye drop, intraocular injection, intravitreal injection, topically, or through the use of a drug eluting device, microcapsule, implant, or microfluidic device. In some cases, the compounds of the present invention are administered with a carrier or excipient that increases the intraocular penetrance of the compound such as an oil and water emulsion with colloid particles having an oily core surrounded by an interfacial film.

In some cases, the colloid particles include at least one cationic agent and at least one non-ionic sufactant such as a poloxamer, tyloxapol, a polysorbate, a polyoxyethylene castor oil derivative, a sorbitan ester, or a polyoxyl stearate. In some cases, the cationic agent is an alkylamine, a tertiary alkyl amine, a quarternary ammonium compound, a cationic lipid, an amino alcohol, a biguanidine salt, a cationic compound or a mixture thereof. In some cases the cationic agent is a biguanidine salt such as chlorhexidine, polyaminopropyl biguanidine, phenformin, alkylbiguanidine, or a mixture thereof. In some cases, the quaternary ammonium compound is a benzalkonium halide, lauralkonium halide, cetrimide, hexadecyltrimethylammonium halide, tetradecyltrimethylammonium halide, dodecyltrimethylammonium halide, cetrimonium halide, benzethonium halide, behenalkonium halide, cetalkonium halide, cetethyldimonium halide, cetylpyridinium halide, benzododecinium halide, chlorallyl methenamine halide, rnyristylalkonium halide, stearalkonium halide or a mixture of two or more thereof. In some cases, cationic agent is a benzalkonium chloride, lauralkonium chloride, benzododecinium bromide, benzethenium chloride, hexadecyltrimethylammonium bromide, tetradecyltrimethylammonium bromide, dodecyltrimethylammonium bromide or a mixture of two or more thereof. In some cases, the oil phase is mineral oil and light mineral oil, medium chain triglycerides (MCT), coconut oil; hydrogenated oils comprising hydrogenated cottonseed oil, hydrogenated palm oil, hydrogenate castor oil or hydrogenated soybean oil; polyoxyethylene hydrogenated castor oil derivatives comprising poluoxyl-40 hydrogenated castor oil, polyoxyl-60 hydrogenated castor oil or polyoxyl-100 hydrogenated castor oil.

In another embodiment, the present invention provides a drug eluting device for treatment of ophthalmic disease. In some cases, the drug eluting device includes one or more compounds of the present invention for slow release. In some cases, the drug eluting device further includes a non-biodegradable polymer from which the one or more compounds are slowly released non-biodegradable compounds include silicone, acrylates, polyethylenes, polyurethane, polyester, polypropylene, polytetrafluoroethylene, poly ether ketone, nylon, collagen, polyethylene terepthalate, polycarbonate, or polyimide. In other cases, the drug eluting device includes a biodegradable polymer from which one or more compounds are slowly released biodegradable polymers include polyglycolide, polylactide, poly ε-caprolactone, polyglyconate, polyhydroxybutyrate, polyhydroxyvalerate, and polydioxanone. In other cases, the drug eluting device is composed of biodegradable and non-biodegradable components. In still other cases, the drug eluting device is composed of a non-ferrous metal suitable for implant into the eye of a subject.

In some of the embodiments of the methods of the invention, the one or more antagonists are of Formula I:

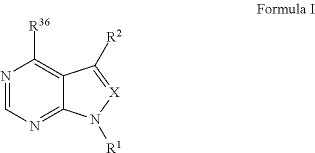

wherein X is ═N— or ═C(H)—; R¹ is hydrogen, R³-substituted or unsubstituted alkyl, R³-substituted or unsubstituted heteroalkyl, R³-substituted or unsubstituted cycloalkyl, R³-substituted or unsubstituted heterocycloalkyl, R³-substituted or unsubstituted aryl, or R³-substituted or unsubstituted heteroaryl; R² is halogen, R⁴-substituted aryl, or substituted or unsubstituted heteroaryl; R³ is halogen, —CN, —OR⁵, —S(O)ₙR⁶, —NR⁷R⁸, —C(O)R⁹, ═N—NH₂, —NR¹⁰—C(O)R¹¹, —NR¹²—C(O)—OR¹³, —C(O)NR¹⁴R¹⁵, —NR¹⁶S(O)₂R¹⁷, —S(O)₂NR¹⁸, R¹⁹-substituted or unsubstituted alkyl, R¹⁹-substituted or unsubstituted heteroalkyl, R¹⁹-substituted or unsubstituted cycloalkyl, R¹⁹-substituted or unsubstituted heterocycloalkyl, R¹⁹-substituted or unsubstituted aryl, or R¹⁹-substituted or unsubstituted heteroaryl, wherein n is an integer from 0 to 2; R³⁶ is —NR³⁷R³⁸; R⁴ is halogen, —CN, —OR²⁰, —S(O)_{q}R²¹, —NR²²R²³, —C(O)R²⁴, ═N—NH₂, —NR²⁵—C(O)R²⁶, —NR²⁷—C(O)—OR²⁸, —C(O)NR²⁹R³⁰, —NR³¹S(O)₂R³², —S(O)₂NR³³, R³⁴-substituted or unsubstituted alkyl, R³⁴substituted or unsubstituted heteroalkyl, R³⁴-substituted or unsubstituted cycloalkyl, R³⁴-substituted or unsubstituted heterocycloalkyl, R³⁴-substituted or unsubstituted aryl, or R³⁴-substituted or unsubstituted heteroaryl, wherein q is an integer from 0 to 2; R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², and R³³ are independently hydrogen, R³⁵-substituted or unsubstituted alkyl, R³⁵-substituted or unsubstituted heteroalkyl, unsubstituted cycloalkyl, R³⁵-substituted or unsubstituted heterocycloalkyl, R³⁵-substituted or unsubstituted aryl, or R³⁵-substituted or unsubstituted heteroaryl; R¹⁹, R³⁴ and R³⁵ are independently hydrogen, halogen, unsubstituted alkyl, unsubstituted heteroalkyl, unsubstituted cycloalkyl, unsubstituted heterocycloalkyl, unsubstituted aryl, or unsubstituted heteroaryl; and R³⁷and R³⁸ are hydrogen, halogen, or unsubstituted alkyl.

In some of the embodiments of the methods of the invention, said one or more antagonists are of Formula XV

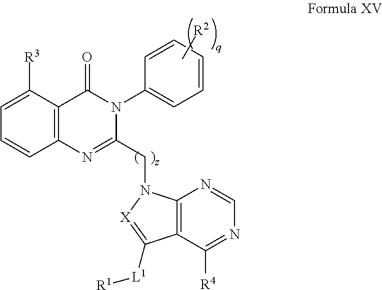

wherein q is an integer from 0 to 5; z is an integer from 0 to 10; X is ═CH— or ═N—; L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; R¹ and R² are independently halogen, —CN, —OR⁵, —S(O)ₙR⁶, —NR⁷R⁸, —C(O)R⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein n is independently an integer from 0 to 2; R³, and R⁴ are independently hydrogen, halogen, —CN, —OR⁵, —S(O)ₙR⁶, —NR⁷R⁸, —C(O)R⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁵ is independently hydrogen, —C(O)R¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶ is independently hydrogen, —NR¹¹R¹², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein if n is 1 or 2 then R⁶ is other than hydrogen; R⁷ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁸ is independently hydrogen, —S(O)ₙR¹³, —C(O)R¹⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁹ is independently —NR¹⁵R¹⁶, hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁰ is independently hydrogen, —NR¹⁷R¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁴ is independently hydrogen, —NR¹⁹R²⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R¹¹, R¹², R¹³, R¹⁵, R¹⁶, R¹⁷, R¹⁸, R¹⁹, and R²⁰ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the PI3K/mTOR pathway.

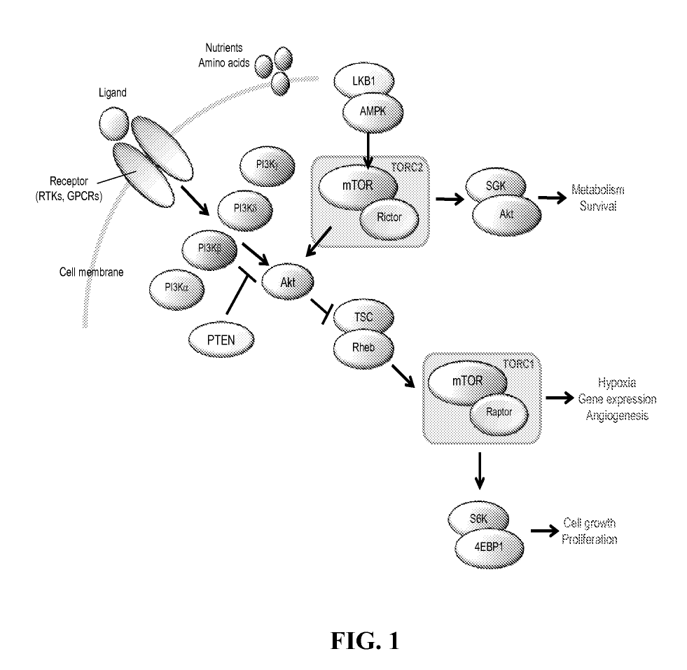

FIG. 2 illustrates a mechanism for angiogenesis involving mTOR activation.

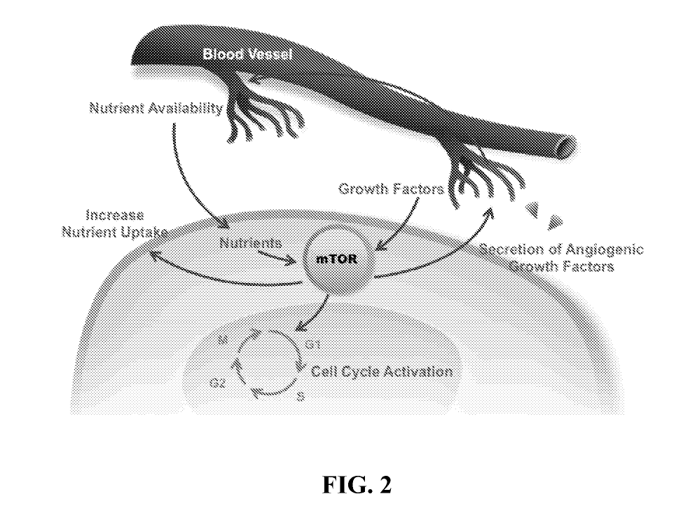

### DETAILED DESCRIPTION OF THE INVENTION

### I. Definitions

Abbreviations used herein have their conventional meaning within the chemical and biological arts.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the appended claims define the scope of the invention and that methods and structures within the scope of these claims and their equivalents be covered thereby.

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” include plural references unless the context clearly dictates otherwise.

The term “effective amount” or “therapeutically effective amount” refers to that amount of a compound described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g. reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating,” or “palliating” or “ameliorating” is used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. The treatment or amelioration of symptoms can be based on objective or subjective parameters; including the results of a physical examination, functional (self) evaluation, and/or any form of vision evaluation. For example, the certain methods presented herein successfully treat vision loss due to aberrant neovascularization or vascular permeability in the eye by decreasing the incidence of angiogenesis and or preventing the incidence of angiogenesis.

The “therapeutic effect” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “co-administration,” “administered in combination with,” and their grammatical equivalents, as used herein, encompass administration of two or more agents to an animal so that both agents and/or their metabolites are present in the animal at the same time. Co-administration includes simultaneous administration in separate compositions, administration at different times in separate compositions, or administration in a composition in which both agents are present.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The terms “antagonist” and “inhibitor” are used interchangeably, and they refer to a compound having the ability to inhibit a biological function of a target protein, whether by inhibiting the activity or expression of the target protein. Accordingly, the terms “antagonist” and “inhibitors” are defined in the context of the biological role of the target protein. While preferred antagonists herein specifically interact with (e.g. bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g. bind to) the target, compounds that initiate or enhance a biological activity of the target polypeptide by interacting with other members of the signal transduction pathway of which the target polypeptide is a member are also specifically included within this definition.

As used herein, “agent” or “biologically active agent” refers to a biological, pharmaceutical, or chemical compound or other moiety. Non-limiting examples include simple or complex organic or inorganic molecule, a peptide, a protein, an oligonucleotide, an antibody, an antibody derivative, antibody fragment, a vitamin derivative, a carbohydrate, a toxin, or a chemotherapeutic compound. Various compounds can be synthesized, for example, small molecules and oligomers (e.g., oligopeptides and oligonucleotides), and synthetic organic compounds based on various core structures. In addition, various natural sources can provide compounds for screening, such as plant or animal extracts, and the like. A skilled artisan can readily recognize that there is no limit as to the structural nature of the agents of the present invention.

“Signal transduction” is a process during which stimulatory or inhibitory signals are transmitted into and within a cell to elicit an intracellular response. A modulator of a signal transduction pathway refers to a compound which modulates the activity of one or more cellular proteins mapped to the same specific signal transduction pathway. A modulator may augment (agonist) or suppress (antagonist) the activity of a signaling molecule.

An “anti-angiogenesis agent”, “anti-angiogenetic agent”, “antiangiogenetic agent”, “anti-angiogenetic drug”, or “anti-angiogenesis drug” refers to any agent useful in the treatment of a condition characterized by excessive or abnormal angiogenesis. “Antiangiogenetic therapy” means the administration of one or more antiangiogenetic drugs and/or other agents to a patient suffering from a disorder characterized by excessive or abnormal angiogenesis by various methods, including intravenous, oral, intramuscular, intraperitoneal, intravesical, subcutaneous, transdermal, buccal, instillation, continuous release, sustained release, ocular inserts, inhalation or in the form of a suppository.

The term “cell proliferation” refers to a phenomenon by which the cell number has changed as a result of division. This term also encompasses cell growth by which the cell morphology has changed (e.g., increased in size) consistent with a proliferative signal.

The term “selective inhibition” or “selectively inhibit” as applied to a biologically active agent refers to the agent's ability to preferentially reduce the target signaling activity as compared to off-target signaling activity, via direct or interact interaction with the target.

As used herein, the term “mTORC1/mTORC2 inhibitor” refers to such compounds that inhibit both mTORC1 and mTORC2. These compounds are distinguished from mTOR inhibitors such as rapamycin and rapamcyin analogues (i.e. rapalogues) due to their ability to inhibit mTORC1 and mTORC2. In some embodiments, the inhibition of both mTORC1 and mTORC2 can be measured by assaying the effects of the compounds of the present invention on the phosphorylation of Akt.

“Subject” refers to an animal, such as a mammal, for example a human. The methods described herein can be useful in both human therapeutics and veterinary applications. In some embodiments, the patient is a mammal, and in some embodiments, the patient is human.

The term “pharmaceutically acceptable salts” is meant to include salts of active compounds which are prepared with relatively nontoxic acids or bases, depending on the particular substituent moieties found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

The term “in vivo” refers to an event that takes place in a subject's body.

The term “in vitro” refers to an event that takes places outside of a subject's body. For example, an in vitro assay encompasses any assay run outside of a subject assay. In vitro assays encompass cell-based assays in which cells alive or dead are employed. In vitro assays also encompass a cell-free assay in which no intact cells are employed.

### Exemplary Compounds Useful in the Methods of the Invention

Description of compounds of the present invention is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

“Acyl” refers to a —(C═O)R radical wherein “R” is alkyl, aryl, heteroaryl, heteroalkyl, or heterocyclyl, which are as described herein. In some embodiments, it is a C₁-C₁₀ acyl radical which refers to the total number of chain or ring atoms of the alkyl, aryl, heteroaryl or heterocyclyl portion of the acyloxy group plus the carbonyl carbon of acyl, i.e three other ring or chain atoms plus carbonyl. If the R radical is heteroaryl or heterocyclyl, the hetero ring or chain atoms contribute to the total number of chain or ring atoms. Unless stated otherwise specifically in the specification, the “R” of an acyloxy group is optionally substituted by one or more substituents which independently are: alkyl, heteroalkyl, alkenyl, alkynyl, cycloalkyl, heterocyclyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Acyloxy” refers to a R(C═O)O— radical wherein “R” is alkyl, aryl, heteroaryl, heteroalkyl, or heterocyclyl, which are as described herein. In some embodiments, it is a C₁-C₄ acyloxy radical which refers to the total number of chain or ring atoms of the alkyl, aryl, heteroaryl or heterocyclyl portion of the acyloxy group plus the carbonyl carbon of acyl, i.e three other ring or chain atoms plus carbonyl. If the R radical is heteroaryl or heterocyclyl, the hetero ring or chain atoms contribute to the total number of chain or ring atoms. Unless stated otherwise specifically in the specification, the “R” of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2 —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Aralkyl” or “arylalkyl” refers to an (aryl)alkyl—radical where aryl and alkyl are as disclosed herein and which are optionally substituted by one or more of the subsituents described as suitable substituents for aryl and alkyl respectively.

“Alkoxy” refers to a (alkyl)O—radical, where alkyl is as described herein and contains 1 to 10 carbons (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms. In some embodiments, it is a C₁-C₄ alkoxy group. A alkoxy moiety may be substituted by one or more of the substituents described as suitable substituents for an alkyl radical.

“Alkyl” refers to a straight or branched hydrocarbon chain radical consisting solely of carbon and hydrogen atoms, containing no unsaturation, having from one to ten carbon atoms (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated. In some embodiments, it is a C₁-C₄ alkyl group. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, and the like. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group is optionally substituted by one or more of substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

The term “alkylene” by itself or as part of another substituent means a divalent radical derived from an alkyl, as exemplified, but not limited, by —CH₂CH₂CH₂CH₂—, —CH₂CH═CHCH₂—, —CH₂C≡CCH₂—, —CH₂CH₂CH(CH₂CH₂CH₃)CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

“Alkylaryl” refers to an -(alkyl)aryl radical where aryl and alkyl are as disclosed herein and which are optionally substituted by one or more of the subsituents described as suitable substituents for aryl and alkyl respectively.

“Alkylhetaryl” refers to an -(alkyl)hetaryl radical where hetaryl and alkyl are as disclosed herein and which are optionally substituted by one or more of the subsituents described as suitable substituents for aryl and alkyl respectively.

“Alkylheterocyclyl” refers to an -(alkyl) heterocycyl radical where alkyl and heterocyclyl are as disclosed herein and which are optionally substituted by one or more of the subsituents described as suitable substituents for heterocyclyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (ie. C₂-C₁₀ alkenyl). Whenever it appears herein, a numerical range such as “2 to 10” refers to each integer in the given range; e.g., “2 to 10 carbon atoms” means that the alkenyl group may consist of 2 carbon atoms, 3 carbon atoms, etc., up to and including 10 carbon atoms. In certain embodiments, an alkenyl comprises two to eight carbon atoms. In other embodiments, an alkenyl comprises two to five carbon atoms (e.g., C₂-C₅ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkenyl-cycloalkyl” refers to an -(alkenyl)cycloalkyl radical where alkenyl and cyclo alkyl are as disclosed herein and which are optionally substituted by one or more of the subsituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (ie. C₂-C₁₀ alkynyl). Whenever it appears herein, a numerical range such as “2 to 10” refers to each integer in the given range; e.g., “2 to 10 carbon atoms” means that the alkynyl group may consist of 2 carbon atoms, 3 carbon atoms, etc., up to and including 10 carbon atoms. In certain embodiments, an alkynyl comprises two to eight carbon atoms. In other embodiments, an alkynyl has two to five carbon atoms (e.g., C₂-C₅ alkynyl). The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkynyl-cycloalkyl” refers to refers to an -(alkynyl)cycloalkyl radical where alkynyl and cyclo alkyl are as disclosed herein and which are optionally substituted by one or more of the subsituents described as suitable substituents for alkynyl and cycloalkyl respectively.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl, unless stated otherwise specifically in the specification. When a —N(R^{a})₂ group has two Ra other than hydrogen they can be combined with the nitrogen atom to form a 4-, 5-, 6-, or 7-membered ring. For example, —N(R^{a})₂ is meant to include, but not be limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon). In some embodiments it is a C₁-C₄ amido or amide radical, which includes the amide carbonyl in the total number of carbons in the radical. The R₂ of —N(R)₂ of the amide may optionally be taken together with the nitrogen to which it is attached to form a 4-, 5-, 6-, or 7-membered ring. Unless stated otherwise specifically in the specification, an amido group is optionally substituted independently by one or more of the substituents as described herein for alkyl, cycloalkyl, aryl, heteroaryl, or heterocyclyl. An amide may be an amino acid or a peptide molecule attached to a compound of Formula (I), thereby forming a prodrug. Any amine, hydroxy, or carboxyl side chain on the compounds described herein can be amidified. The procedures and specific groups to make such amides are known to those of skill in the art and can readily be found in reference sources such as Greene and Wuts, Protective Groups in Organic Synthesis, 3.sup.rd Ed., John Wiley & Sons, New York, N.Y., 1999, which is incorporated herein by reference in its entirety.

“Aromatic” or “aryl” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (ie. C₂-C₁₀ cycloalkyl). Whenever it appears herein, a numerical range such as “3 to 10” refers to each integer in the given range; e.g., “3 to 10 carbon atoms” means that the cycloalkyl group may consist of 3 carbon atoms, etc., up to and including 10 carbon atoms. In some embodiments, it is a C₃-C₈ cycloalkyl radical. In some embodiments, it is a C₃-0₅ cycloalkyl radical. Illustrative examples of cycloalkyl groups include, but are not limited to the following moieties: cyclopropyl, cyclobutyl, cyclopentyl, cyclohexyl, cycloseptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Cycloalkyl-alkenyl” refers to a -(cycloalkyl) alkenyl radical where cycloalkyl and heterocyclyl are as disclosed herein and which are optionally substituted by one or more of the subsituents described as suitable substituents for heterocyclyl and cycloalkyl respectively.

“Cycloalkyl-heterocyclyl” refers to a -(cycloalkyl) heterocycyl radical where cycloalkyl and heterocyclyl are as disclosed herein and which are optionally substituted by one or more of the subsituents described as suitable substituents for heterocyclyl and cycloalkyl respectively.

“Cycloalkyl-heteroaryl” refers to a -(cycloalkyl) heteroaryl radical where cycloalkyl and heterocyclyl are as disclosed herein and which are optionally substituted by one or more of the subsituents described as suitable substituents for heterocyclyl and cycloalkyl respectively

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). Any amine, hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3.sup.rd Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—Ra, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given, e.g. C₁-C₄ heteroalkyl which refers to the chain length in total, which in this example is 4 atoms long. For example, a —CH₂OCH₂CH₃ radical is referred to as a “C₄” heteroalkyl, which includes the heteroatom center in the atom chain length description. Connection to the rest of the molecule may be through either a heteroatom or a carbon in the heteroalkyl chain. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Heteroalkylaryl” refers “to an -(heteroalkyl)aryl radical where heteroalkyl and aryl are as disclosed herein and which are optionally substituted by one or more of the subsituents described as suitable substituents for heteroalkyl and aryl respectively.

“Heteroalkylheteroaryl” refers “to an -(heteroalkyl)heteroaryl radical where heteroalkyl and heteroaryl are as disclosed herein and which are optionally substituted by one or more of the subsituents described as suitable substituents for heteroalkyl and heteroaryl respectively

“Heteroalkylheterocyclyl” refers “to an -(heteroalkyl)heterocyclyl radical where heteroalkyl and heteroaryl are as disclosed herein and which are optionally substituted by one or more of the subsituents described as suitable substituents for heteroalkyl and heterocyclyl respectively

“Heteroalkylcycloalkyl” refers “to an -(heteroalkyl) cycloalkyl radical where heteroalkyl and cycloalkyl are as disclosed herein and which are optionally substituted by one or more of the subsituents described as suitable substituents for heteroalkyl and cycloalkyl respectively

“Heteroaryl” or, alternatively, “heteroaromatic” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range; e.g., “5 to 18 ring atoms”, means that the heteroaryl group may consist of 5 ring atoms, 6 ring atoms, etc., up to and including 18 ring atoms. An N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b] [1,4]dioxepinyl, benzo[b] [1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl,isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, a heteraryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Heteroarylalkyl” or “hetarylalkyl” refers to an (heteroaryl)alkyl—radical where heteroaryl and alkyl are as disclosed herein and which are optionally substituted by one or more of the subsituents described as suitable substituents for aryl and alkyl respectively.

“ Heterocycloalkyl” or “heterocyclylalkyl” refers to a radical of the formula —R^{c}-heterocyclyl where R^{c} is an alkyl chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclyl” refers to a stable 3- to 18-membered non-aromatic ring (e.g., C₃-C₁₈ heterocyclyl) radical that comprises two to twelve carbon atoms and from one to six heteroatoms selected from nitrogen, oxygen and sulfur. Whenever it appears herein, a numerical range such as “3 to 18” refers to each integer in the given range; e.g., “3 to 18 ring atoms” means that the heteroaryl group may consist of 3 ring atoms, 4 ring atoms, etc., up to and including 18 ring atoms. In some embodiments, it is a C₅-C₁₀ heterocyclyl. In some embodiments, it is a C₄-C₁₀heterocyclyl. In some embodiments, it is a C₃-C₁₀heterocyclyl. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocylyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a}C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(Rₐ)₂, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Heteroalicyclic” refers to a cycloalkyl radical that includes at least one heteroatom selected from nitrogen, oxygen and sulfur. The radicals may be fused with an aryl or heteroaryl. The term heteroalicyclic also includes all ring forms of the carbohydrates, including but not limited to the monosaccharides, the disaccharides and the oligosaccharides. Unless stated otherwise specifically in the specification, a heteroalicyclic group is optionally substituted by one or more of substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —n(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Imino” refers to the ═N—H radical.

“Isocyanato” refers to a —NCO radical.

“Isothiocyanato” refers to a —NCS radical.

“Mercaptyl” refers to a (alkyl)S or (H)S radical.

“Moiety” refers to a specific segment or functional group of a molecule. Chemical moieties are often recognized chemical entities embedded in or appended to a molecule.

“Nitro” refers to the —NO₂ radical.

“Oxa” refers to the —O— radical.

“Oxo” refers to the ═O radical.

“Sulfinyl” refers to a —S(═O)—R radical, where R is selected from the group consisting of alkyl, cycloalkyl, aryl, heteroaryl (bonded through a ring carbon) and heteroalicyclic (bonded through a ring carbon).

“Sulfonyl” refers to a —S(═O)₂—R radical, where R is selected from the group consisting of alkyl, cycloalkyl, aryl, heteroaryl (bonded through a ring carbon) and heteroalicyclic (bonded through a ring carbon).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6-, or 7-membered ring. In some embodiments, it is a C₁-C₁₀ sulfonamido, wherein each R in sulfonamido contains 1 carbon, 2 carbons, 3 carbons, or 4 carbons total. A sulfonamido group is optionally substituted by one or more of the subsituents described for alkyl, cycloalkyl, aryl, heteroaryl respectively

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl respectively.

“Thiocyanato” refers to a —CNS radical.

“Thioxo” refers to the ═S radical.

“Substituted” means that the referenced group may be substituted with one or more additional group(s) individually and independently selected from acyl, alkyl, alkylaryl, cycloalkyl, aralkyl, aryl, carbohydrate, heteroaryl, heterocyclic, hydroxy, alkoxy, aryloxy, mercapto, alkylthio, arylthio, cyano, halo, carbonyl, ester, thiocarbonyl, isocyanato, thiocyanato, isothiocyanato, nitro, perhaloalkyl, perfluoroalkyl, phosphate, silyl, sulfonyl, sulfonyl, sulfonamidyl, sulfoxyl, sulfonate, and amino, including mono- and di-substituted amino groups, and the protected derivatives thereof. The subsituents themselves may be substituted, for example, a cycloakyl substituent may have a halide substituted at one or more ring carbons, and the like. The protecting groups that may form the protective derivatives of the above substituents are known to those of skill in the art and may be found in references such as Greene and Wuts, above.

A “size-limited substituent” or “ size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “ lower substituent group,” as used herein means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The compounds presented herein may possess one or more chiral centers and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of compounds having the structure of Formula (I), as well as active metabolites of these compounds having the same type of activity. In addition, the compounds described herein can exist in unsolvated as well as solvated forms with pharmaceutically acceptable solvents such as water, ethanol, and the like. The solvated forms of the compounds presented herein are also considered to be disclosed herein.

Compounds described can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formula I is shown without a definitive stereochemistry at certain positions. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The present invention includes all manner of rotamers and conformationally restricted states of a compound of the invention.

This invention pertains to the use of a distinct class of biologically active agents that exhibit selective inhibition of certain protein kinases, and the uses of these agents for treatment of diseases mediated by such protein kinases. In various embodiments, the present invention provides methods of treating ocular disorders by inhibiting angiogenesis. In one embodiment, the invention provides a method for inhibiting angiogenesis by administering an inhibitor of one or more type I phosphatidylinositol 3 -kinases (PI3-kinase), wherein the one or more type I PI3-kinase is selected from the group consisting of PI3-kinase α, PI3-kinase β, PI3-kinase γ, and PI3-kinase δ. In another embodiment, the method for inhibiting angiogenesis includes administration of a selective inhibitor of one or more type I PI3-kinase selected from the group consisting of PI3-kinase α, PI3-kinase β, PI3-kinase γ, and PI3-kinase δ. In another embodiment, the present invention provides a method for inhibiting angiogenesis comprising contacting a cell with a biologically active agent that selectively inhibits mTorC1 and/or mTorC2 activity relative to one or more type I phosphatidylinositol 3-kinases (PI3-kinase), wherein the one or more type I PI3-kinase is selected from the group consisting of PI3-kinase α, PI3-kinase β, PI3-kinase γ, and PI3-kinase δ. In another embodiment, the present invention provides a method for inhibiting angiogenesis comprising contacting a cell with a biologically active agent that inhibits mTorC1 and/or mTorC2 activity as well as one or more type I phosphatidylinositol 3-kinases (PI3-kinase), wherein the one or more type I PI3-kinase is selected from the group consisting of PI3-kinase α, PI3-kinase β, PI3-kinase γ, and PI3-kinase δ.

In one aspect, the invention provides a kinase antagonist which is a compound of Formula I, or a pharmaceutically acceptable salt thereof:

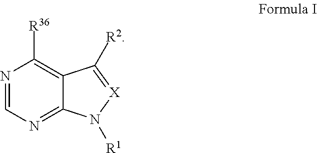

In Formula (I), X is ═N— or ═C(H)—.

R¹ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, when R¹ is substituted alkyl, substituted heteroalkyl, substituted cycloalkyl, substituted heterocycloalkyl, substituted aryl, or substituted heteroaryl, it is substituted by R³.

R² is halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, when R² is substituted aryl or substituted heteroaryl, it is R⁴-substituted aryl, or R⁴-substituted or unsubstituted heteroaryl. In some embodiments, when R² is substituted aryl, it is R⁴-substituted aryl.

R³ is halogen, —CN, —OR⁵, —S(O)ₙR⁶, —NR⁷R⁸, —C(O)R⁹, ═N—NH₂, —NR¹⁰—C(O)R¹¹, —NR¹²—C(O)—OR¹³, —C(O)NR¹⁴R¹⁵, —NR¹⁶S(O)2R17, —S(O)₂NR¹⁸, R¹⁹-substituted or unsubstituted alkyl, R¹⁹-substituted or unsubstituted heteroalkyl, R¹⁹-substituted or unsubstituted cycloalkyl, R¹⁹-substituted or unsubstituted heterocycloalkyl, R¹⁹-substituted or unsubstituted aryl, or R¹⁹-substituted or unsubstituted heteroaryl. The symbol n is an integer from 0 to 2.

R⁴ is halogen, —CN, —OR²⁰, —S(O)_{q}R²¹, —NR²²R²³, —C(O)R²⁴, —C(O)OR²⁴, ═N—NH₂, —NR²⁵—C(O)R²⁶, —NR²⁷—C(O)—OR²⁸, —C(O)NR²⁹R³⁰, —NR³¹S(O)₂R³², —S(O)₂NR³³, R³⁴-substituted or unsubstituted alkyl, R³⁴-substituted or unsubstituted heteroalkyl, R³⁴-substituted or unsubstituted cycloalkyl, R³⁴-substituted or unsubstituted heterocycloalkyl, R³⁴-substituted or unsubstituted aryl, or R³⁴-substituted or unsubstituted heteroaryl. The symbol q represents an integer from 0 to 2.

R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², and R³³ are independently hydrogen, R³⁵-substituted or unsubstituted alkyl, R³⁵-substituted or unsubstituted heteroalkyl, unsubstituted cycloalkyl, R³⁵-substituted or unsubstituted heterocycloalkyl, R³⁵-substituted or unsubstituted aryl, or R³⁵-substituted or unsubstituted heteroaryl.

R¹⁹, R³⁴ and R³⁵ are independently hydrogen, halogen, unsubstituted alkyl, unsubstituted heteroalkyl, unsubstituted cycloalkyl, unsubstituted heterocycloalkyl, unsubstituted aryl, or unsubstituted heteroaryl.

R³⁶ is halogen, —NR³⁷R³⁸, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³⁷ and R³⁸ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments X is ═N—. In some embodiments X is ═C(H)—.

In some embodiments, R¹ is hydrogen. In some embodiments, R¹ is halogen. In some embodiments, R¹ is unsubstituted alkyl. In some embodiments, R¹ is substituted alkyl. In some embodiments, R¹ is unsubstituted heteroalkyl. In some embodiments, R¹ is substituted heteroalkyl. In some embodiments, R¹ is unsubstituted cycloalkyl. In some embodiments, R¹ is substituted cycloalkyl. In some embodiments, R¹ is unsubstituted heterocycloalkyl. In some embodiments, R¹ is substituted heterocycloalkyl. In some embodiments, R¹ is unsubstituted aryl. In some embodiments, R¹ is substituted aryl. In some embodiments, R¹ is unsubstituted heteroaryl. In some embodiments, R¹ is substituted heteroaryl.

In some embodiments, when R¹ is substituted alkyl, it is substituted by halogen. In some embodiments, when R¹ is substituted alkyl, it is substituted by —CN. In some embodiments, when R¹ is substituted alkyl, it is substituted by —OR⁵. In some embodiments, when R¹ is substituted alkyl, it is substituted by —S(O),_{I}R⁶. In some embodiments, when R¹ is substituted alkyl, it is substituted by —NR⁷R⁸. In some embodiments, when R¹ is substituted alkyl, it is substituted by —C(O)R⁹. In some embodiments, when R¹ is substituted alkyl, it is substituted by —NR⁷R⁸. In some embodiments, when R¹ is substituted alkyl, it is substituted by ═N—NH₂. In some embodiments, when R ¹ is substituted alkyl, it is substituted by —NR¹⁰—C(O)R¹¹. In some embodiments, when R¹ is substituted alkyl, it is substituted by —NR¹²—C(O)—OR¹³. In some embodiments, when R¹ is substituted alkyl, it is substituted by —C(O)NR¹⁴R¹⁵. In some embodiments, when R¹ is substituted alkyl, it is substituted by —NR¹⁶S(O)₂R¹⁷. In some embodiments, when R¹ is substituted alkyl, it is substituted by —S(O)₂NR¹⁸. In some embodiments, when R¹ is substituted alkyl, it is substituted by R¹⁹-substituted alkyl. In some embodiments, when R¹ is substituted alkyl, it is substituted by unsubstituted alkyl. In some embodiments, when R¹ is substituted alkyl, it is substituted by R¹⁹-substituted heteroalkyl. In some embodiments, when R¹ is substituted alkyl, it is substituted by unsubstituted heteroalkyl. In some embodiments, when R¹ is substituted alkyl, it is substituted by R¹⁹-substituted cycloalkyl. In some embodiments, when R¹ is substituted alkyl, it is substituted by unsubstituted cycloalkyl. In some embodiments, when R¹ is substituted alkyl, it is substituted by R¹⁹-substituted heterocycloalkyl. In some embodiments, when R¹ is substituted alkyl, it is substituted by unsubstituted heterocycloalkyl. In some embodiments, when R¹ is substituted alkyl, it is substituted by R¹⁹-substituted aryl. In some embodiments, when R¹ is substituted alkyl, it is substituted by unsubstituted aryl. In some embodiments, when R¹ is substituted alkyl, it is substituted by R¹⁹-substituted heteroaryl. In some embodiments, when R¹ is substituted alkyl, it is substituted by unsubstituted heteroaryl.

In some embodiments, when R¹ is substituted heteroalkyl, it is substituted by halogen. In some embodiments, when R¹ is substituted heteroalkyl, it is substituted by —CN. In some embodiments, when R¹ is substituted heteroalkyl, it is substituted by —OR⁵. In some embodiments, when R¹ is substituted heteroalkyl, it is substituted by —S(O)ₙR⁶. In some embodiments, when R¹ is substituted heteroalkyl, it is substituted by —NR⁷R⁸. In some embodiments, when R¹ is substituted heteroalkyl, it is substituted by —C(O)R⁹. In some embodiments, when R¹ is substituted heteroalkyl, it is substituted by —NR⁷R⁸. In some embodiments, when R¹ is substituted heteroalkyl, it is substituted by ═N—NH₂. In some embodiments, when R¹ is substituted heteroalkyl, it is substituted by —NR¹⁰—C(O)R¹¹. In some embodiments, when R¹ is substituted heteroalkyl, it is substituted by —NR¹²—C(O)—OR¹³. In some embodiments, when R¹ is substituted heteroalkyl, it is substituted by —C(O)NR¹⁴R¹⁵. In some embodiments, when R¹ is substituted heteroalkyl, it is substituted by —NR¹⁶S(O)₂R¹⁷. In some embodiments, when R¹ is substituted heteroalkyl, it is substituted by —S(O)₂NR¹⁸. In some embodiments, when R¹ is substituted heteroalkyl, it is substituted by R¹⁹-substituted alkyl. In some embodiments, when R¹ is substituted heteroalkyl, it is substituted by unsubstituted alkyl. In some embodiments, when R¹ is substituted heteroalkyl, it is substituted by R¹⁹-substituted heteroalkyl. In some embodiments, when R¹ is substituted heteroalkyl, it is substituted by unsubstituted heteroalkyl. In some embodiments, when R¹ is substituted heteroalkyl, it is substituted by R¹⁹-substituted cycloalkyl. In some embodiments, when R¹ is substituted heteroalkyl, it is substituted by unsubstituted cycloalkyl. In some embodiments, when R¹ is substituted heteroalkyl, it is substituted by R¹⁹-substituted heterocycloalkyl. In some embodiments, when R¹ is substituted heteroalkyl, it is substituted by unsubstituted heterocycloalkyl. In some embodiments, when R¹ is substituted heteroalkyl, it is substituted by R¹⁹-substituted aryl. In some embodiments, when R¹ is substituted heteroalkyl, it is substituted by unsubstituted aryl. In some embodiments, when R¹ is substituted heteroalkyl, it is substituted by R¹⁹-substituted heteroaryl. In some embodiments, when R¹ is substituted heteroalkyl, it is substituted by unsubstituted heteroaryl.

In some embodiments, when R¹ is substituted cycloalkyl, it is substituted by halogen. In some embodiments, when R¹ is substituted cycloalkyl, it is substituted by —CN. In some embodiments, when R¹ is substituted cycloalkyl, it is substituted by —OR⁵. In some embodiments, when R¹ is substituted cycloalkyl, it is substituted by —S(O),_{I}R⁶. In some embodiments, when R¹ is substituted cycloalkyl, it is substituted by —NR⁷R⁸. In some embodiments, when R¹ is substituted cycloalkyl, it is substituted by —C(O)R⁹. In some embodiments, when R¹ is substituted cycloalkyl, it is substituted by —NR⁷R⁸. In some embodiments, when R¹ is substituted cycloalkyl, it is substituted by ═N—NH₂. In some embodiments, when R¹ is substituted cycloalkyl, it is substituted by —NR¹⁰—C(O)R¹¹. In some embodiments, when R¹ is substituted cycloalkyl, it is substituted by —NR¹²—C(O)—OR¹³. In some embodiments, when R¹ is substituted cycloalkyl 1, it is substituted by —C(O)NR¹⁴R¹⁵. In some embodiments, when R¹ is substituted cycloalkyl, it is substituted by —NR¹⁶S(O)₂R¹⁷. In some embodiments, when R¹ is substituted cycloalkyl, it is substituted by —S(O)₂NR¹⁸. In some embodiments, when R¹ is substituted cycloalkyl, it is substituted by R¹⁹-substituted alkyl. In some embodiments, when R¹ is substituted cycloalkyl, it is substituted by unsubstituted alkyl. In some embodiments, when R¹ is substituted cycloalkyl, it is substituted by R¹⁹-substituted heteroalkyl. In some embodiments, when R¹ is substituted cycloalkyl, it is substituted by unsubstituted heteroalkyl. In some embodiments, when R¹ is substituted cycloalkyl, it is substituted by R¹⁹-substituted cycloalkyl. In some embodiments, when R¹ is substituted cycloalkyl, it is substituted by unsubstituted cycloalkyl. In some embodiments, when R¹ is substituted cycloalkyl, it is substituted by R¹⁹-substituted heterocycloalkyl. In some embodiments, when R¹ is substituted cycloalkyl, it is substituted by unsubstituted heterocycloalkyl. In some embodiments, when R¹ is substituted cycloalkyl, it is substituted by R¹⁹-substituted aryl. In some embodiments, when R¹ is substituted cycloalkyl, it is substituted by unsubstituted aryl. In some embodiments, when R¹ is substituted cycloalkyl, it is substituted by R¹⁹-substituted heteroaryl. In some embodiments, when R¹ is substituted cycloalkyl, it is substituted by unsubstituted heteroaryl.

In some embodiments, when R¹ is substituted heterocycloalkyl, it is substituted by halogen. In some embodiments, when R¹ is substituted heterocycloalkyl, it is substituted by CN. In some embodiments, when R¹ is substituted heterocycloalkyl, it is substituted by —OR⁵. In some embodiments, when R¹ is substituted heterocycloalkyl, it is substituted by —S(O)ₙR⁶. In some embodiments, when R¹ is substituted heterocycloalkyl, it is substituted by —NR⁷R⁸. In some embodiments, when R¹ is substituted heterocycloalkyl, it is substituted by —C(O)R⁹. In some embodiments, when R¹ is substituted heterocycloalkyl, it is substituted by —NR⁷R⁸. In some embodiments, when R¹ is substituted heterocycloalkyl, it is substituted by ═N—NH₂. In some embodiments, when R¹ is substituted heterocycloalkyl, it is substituted by —NR¹⁰—C(O)R¹¹. In some embodiments, when R¹ is substituted heterocycloalkyl, it is substituted by —NR¹²—C(O)—OR¹³. In some embodiments, when R¹ is substituted heterocycloalkyl, it is substituted by —C(O)NR¹⁴R¹⁵. In some embodiments, when R¹ is substituted heterocycloalkyl, it is substituted by —NR¹⁶S(O)₂R¹⁷. In some embodiments, when R¹ is substituted heterocycloalkyl, it is substituted by —S(O)₂NR¹⁸. In some embodiments, when R¹ is substituted heterocycloalkyl, it is substituted by R¹⁹-substituted alkyl. In some embodiments, when R¹ is substituted heterocycloalkyl, it is substituted by unsubstituted alkyl. In some embodiments, when R¹ is substituted heterocycloalkyl, it is substituted by R¹⁹-substituted heteroalkyl. In some embodiments, when R¹ is substituted heterocycloalkyl, it is substituted by unsubstituted heteroalkyl. In some embodiments, when R¹ is substituted heterocycloalkyl, it is substituted by R¹⁹-substituted cycloalkyl. In some embodiments, when R¹ is substituted heterocycloalkyl, it is substituted by unsubstituted cycloalkyl. In some embodiments, when R¹ is substituted heterocycloalkyl, it is substituted by R¹⁹-substituted heterocycloalkyl. In some embodiments, when R¹ is substituted heterocycloalkyl, it is substituted by unsubstituted heterocycloalkyl. In some embodiments, when R¹ is substituted heterocycloalkyl, it is substituted by R¹⁹-substituted aryl. In some embodiments, when R¹ is substituted heterocycloalkyl, it is substituted by unsubstituted aryl. In some embodiments, when R¹ is substituted heterocycloalkyl, it is substituted by R¹⁹-substituted heteroaryl. In some embodiments, when R¹ is substituted heterocycloalkyl, it is substituted by unsubstituted heteroaryl.

In some embodiments, when R¹ is substituted aryl, it is substituted by halogen. In some embodiments, when R¹ is substituted aryl, it is substituted by —CN. In some embodiments, when R¹ is substituted aryl, it is substituted by —OR⁵. In some embodiments, when R¹ is substituted aryl, it is substituted by —S(O)ₙR⁶. In some embodiments, when R¹ is substituted aryl, it is substituted by —NR⁷R⁸. In some embodiments, when R¹ is substituted aryl, it is substituted by —C(O)R⁹. In some embodiments, when R¹ is substituted aryl, it is substituted by —NR⁷R⁸. In some embodiments, when R¹ is substituted aryl, it is substituted by ═N—NH₂. In some embodiments, when R¹ is substituted aryl, it is substituted by —NR¹⁰—C(O)R¹¹. In some embodiments, when R¹ is substituted aryl, it is substituted by —NR¹²—C(O)—OR¹³. In some embodiments, when R¹ is substituted aryl, it is substituted by —C(O)NR¹⁴R¹⁵. In some embodiments, when R¹ is substituted aryl, substituted by —NR¹⁶S(O)₂R¹⁷. In some embodiments, when R¹ is substituted aryl, it is substituted by —S(O)₂NR¹⁸. In some embodiments, when R¹ is substituted aryl, it is substituted by R¹⁹-substituted alkyl. In some embodiments, when R¹ is substituted aryl, it is substituted by unsubstituted alkyl. In some embodiments, when R¹ is substituted aryl, it is substituted by R¹⁹-substituted heteroalkyl. In some embodiments, when R¹ is substituted aryl, it is substituted by unsubstituted heteroalkyl. In some embodiments, when R¹ is substituted aryl, it is substituted by R¹⁹-substituted cycloalkyl. In some embodiments, when R¹ is substituted aryl, it is substituted by unsubstituted cycloalkyl. In some embodiments, when R¹ is substituted aryl, it is substituted by R¹⁹-substituted heterocycloalkyl. In some embodiments, when R¹ is substituted aryl, it is substituted by unsubstituted heterocycloalkyl. In some embodiments, when R¹ is substituted aryl, it is substituted by R¹⁹-substituted aryl. In some embodiments, when R¹ is substituted aryl, it is substituted by unsubstituted aryl. In some embodiments, when R¹ is substituted aryl, it is substituted by R¹⁹-substituted heteroaryl. In some embodiments, when R¹ is substituted aryl, it is substituted by unsubstituted heteroaryl.

In some embodiments, when R¹ is substituted heteroaryl, it is substituted by halogen. In some embodiments, when R¹ is substituted heteroaryl, it is substituted by —CN. In some embodiments, when R¹ is substituted heteroaryl, it is substituted by —OR⁵. In some embodiments, when R¹ is substituted heteroaryl, it is substituted by —S(O)ₙR⁶. In some embodiments, when R¹ is substituted heteroaryl, it is substituted by —NR⁷R⁸. In some embodiments, when R¹ is substituted heteroaryl, it is substituted by —C(O)R⁹. In some embodiments, when R¹ is substituted heteroaryl 1, it is substituted by —NR⁷R⁸. In some embodiments, when R¹ is substituted heteroaryl, it is substituted by ═N—NH₂. In some embodiments, when R¹ is substituted heteroaryl, it is substituted by —NR¹⁰—C(O)R¹¹. In some embodiments, when R¹ is substituted heteroaryl, it is substituted by —NR¹²—C(O)—OR¹³. In some embodiments, when R¹ is substituted heteroaryl, it is substituted by —C(O)NR¹⁴R¹⁵In some embodiments, when R¹ is substituted heteroaryl, it is substituted by —NR¹⁶S(O)₂R¹⁷. In some embodiments, when R¹ is substituted heteroaryl, it is substituted by —S(O)₂NR¹⁸. In some embodiments, when R¹ is substituted heteroaryl, it is substituted by R¹⁹-substituted alkyl. In some embodiments, when R¹ is substituted heteroaryl, it is substituted by unsubstituted alkyl. In some embodiments, when R¹ is substituted heteroaryl, it is substituted by R¹⁹-substituted heteroalkyl. In some embodiments, when R¹ is substituted heteroaryl, it is substituted by unsubstituted heteroalkyl. In some embodiments, when R¹ is substituted heteroaryl, it is substituted by R¹⁹-substituted cycloalkyl. In some embodiments, when R¹ is substituted heteroaryl, it is substituted by unsubstituted cycloalkyl. In some embodiments, when R¹ is substituted heteroaryl, it is substituted by R¹⁹-substituted heterocycloalkyl. In some embodiments, when R¹ is substituted heteroaryl, it is substituted by unsubstituted heterocycloalkyl. In some embodiments, when R¹ is substituted heteroaryl, it is substituted by R¹⁹-substituted aryl. In some embodiments, when R¹ is substituted heteroaryl, it is substituted by unsubstituted aryl. In some embodiments, when R¹ is substituted heteroaryl, it is substituted by R¹⁹-substituted heteroaryl. In some embodiments, when R¹ is substituted heteroaryl, it is substituted by unsubstituted heteroaryl.

In some embodiments, R² is halogen. In some embodiments, R² is unsubstituted alkyl. In some embodiments, R² is substituted alkyl. In some embodiments, R² is unsubstituted heteroalkyl. In some embodiments, R² is substituted heteroalkyl. In some embodiments, R² is unsubstituted cycloalkyl. In some embodiments, R² is substituted cycloalkyl. In some embodiments, R² is unsubstituted heterocycloalkyl. In some embodiments, R² is substituted heterocycloalkyl. In some embodiments, R² is unsubstituted aryl. In some embodiments, R² is substituted aryl. In some embodiments, R² is unsubstituted heteroaryl. In some embodiments, R² is substituted heteroaryl.

In some embodiments, when R² is substituted aryl, it is substituted by halogen, In some embodiments, when R² is substituted aryl, it is substituted by -CN. In some embodiments, when R² is substituted aryl, it is substituted by —OR²⁰. In some embodiments, when R² is substituted aryl, it is substituted by —S(O)_{q}R²¹, where the symbol q represents an integer from 0 to 2 In some embodiments, when R² is substituted aryl, it is substituted by —NR²²R²³. In some embodiments, when R² is substituted aryl, it is substituted by —C(O)R²⁴. In some embodiments, when R² is substituted aryl, it is substituted by ═N—NH₂. In some embodiments, when R² is substituted aryl, it is substituted by —NR²⁵—C(O)R²⁶. In some embodiments, when R² is substituted aryl, it is substituted by —NR²⁷—C(O)—OR²⁸. In some embodiments, when R² is subs aryl, it is substituted by —C(O)NR²⁹R³⁰. In some embodiments, when R² is substituted aryl, it is substituted by —NR³¹S(O)₂R³². In some embodiments, when R² is substituted aryl, it is substituted by —S(O)₂NR³³. In some embodiments, when R² is substituted aryl, it is substituted by R³⁴-substituted alkyl. In some embodiments, when R² is substituted aryl, it is substituted by unsubstituted alkyl. In some embodiments, when R² is substituted aryl, it is substituted by R³⁴-substituted or unsubstituted heteroalkyl. In some embodiments, when R² is substituted aryl, it is substituted by R³⁴-substituted heteroalkyl. In some embodiments, when R² is substituted aryl, it is substituted by unsubstituted heteroalkyl. In some embodiments, when R² is substituted aryl, it is substituted by R³⁴-substituted cycloalkyl. In some embodiments, when R² is substituted aryl, it is substituted by unsubstituted cycloalkyl. In some embodiments, when R² is substituted aryl, it is substituted by R³⁴-substituted heterocycloalkyl. In some embodiments, when R² is substituted aryl, it is substituted by unsubstituted heterocycloalkyl. In some embodiments, when R² is substituted aryl, it is substituted by R³⁴-substituted aryl. In some embodiments, when R² is substituted aryl, it is substituted by unsubstituted aryl. In some embodiments, when R² is substituted aryl, it is substituted by R³⁴-substituted heteroaryl. In some embodiments, when R² is substituted aryl, it is substituted by unsubstituted heteroaryl.

In some embodiments, R³⁶ is halogen. In some embodiments, R³⁶ is —NR³⁷R³⁸. In some embodiments, R³⁶ is unsubstituted alkyl. In some embodiments, R³⁶ is substituted alkyl. In some embodiments, R³⁶ is unsubstituted heteroalkyl. In some embodiments, R³⁶ is substituted heteroalkyl. In some embodiments, R³⁶ is unsubstituted cycloalkyl. In some embodiments, R³⁶ is substituted cycloalkyl. In some embodiments, R³⁶ is unsubstituted heterocycloalkyl. In some embodiments, R³⁶ is substituted heterocycloalkyl. In some embodiments, R³⁶ is unsubstituted aryl. In some embodiments, R³⁶ is substituted aryl. In some embodiments, R³⁶ is unsubstituted heteroaryl. In some embodiments, R³⁶ is substituted heteroaryl.

In some embodiments, R³⁷ and R³⁸ are independently hydrogen, or unsubstituted alkyl.

In some embodiments, R³⁶ is —NH₂, and the kinase antagonist is of the formula:

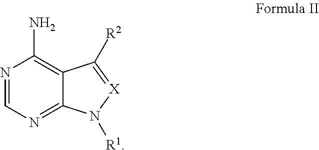

In some embodiments, R¹, R², and X are as defined above in Formula I. In various embodiments, X is ═N—.

In some embodiments of Formulae I and II, R¹ is hydrogen, R³-substituted or unsubstituted alkyl, R³-substituted or unsubstituted heteroalkyl, R³-substituted or unsubstituted cycloalkyl, R³-substituted or unsubstituted heterocycloalkyl, R³-substituted or unsubstituted aryl, or R³-substituted or unsubstituted heteroaryl. R² is halogen, R⁴-substituted aryl, or substituted or unsubstituted heteroaryl.

R³ is halogen, —CN, —OR⁵, —S(O)ₙR⁶, —NR⁷R⁸, —C(O)R⁹, ═N—NH₂, —NR¹⁰—C(O)R¹¹, —NR¹²—C(O)—OR¹³, —C(O)NR¹⁴R¹⁵, —NR¹⁶S(O)₂R¹⁷, —S(O)₂NR¹⁸, R¹⁹-substituted or unsubstituted alkyl, R¹⁹-substituted or unsubstituted heteroalkyl, R¹⁹-substituted or unsubstituted cycloalkyl, R¹⁹-substituted or unsubstituted heterocycloalkyl, R¹⁹-substituted or unsubstituted aryl, or R¹⁹-substituted or unsubstituted heteroaryl. Then symbol n is an integer from 0 to 2.

R⁴ is halogen, —CN, —OR²⁰, —S(O)_{q}R²¹, —NR²²R²³, —C(O)R²⁴, —C(O)OR²⁴, ═N—NH₂, —NR²⁵—C(O)R²⁶, —NR²⁷—C(O)—OR²⁸, —C(O)NR²⁹R³⁰, —NR³¹S(O)₂R³², —S(O)₂NR³³, R³⁴-substituted or unsubstituted alkyl, R³⁴-substituted or unsubstituted heteroalkyl, R³⁴-substituted or unsubstituted cycloalkyl, R³⁴-substituted or unsubstituted heterocycloalkyl, R³⁴-substituted or unsubstituted aryl, or R³⁴-substituted or unsubstituted heteroaryl. The symbol q represents an integer from 0 to 2.

R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², and R³³ are independently hydrogen, R³⁵-substituted or unsubstituted alkyl, R³⁵-substituted or unsubstituted heteroalkyl, unsubstituted cycloalkyl, R³⁵-substituted or unsubstituted heterocycloalkyl, R³⁵-substituted or unsubstituted aryl, or R³⁵-substituted or unsubstituted heteroaryl.

R¹⁹, R³⁴ and R³⁵ are independently hydrogen, halogen, unsubstituted alkyl, unsubstituted heteroalkyl, unsubstituted cycloalkyl, unsubstituted heterocycloalkyl, unsubstituted aryl, or unsubstituted heteroaryl.

In some embodiments, R⁵, R⁶, R⁷, R⁸, R⁹, R¹⁰, R¹¹, R¹², R¹³, R¹⁴, R¹⁵, R¹⁶, R¹⁷, R¹⁸, R²⁰, R²¹, R²², R²³, R²⁴, R²⁵, R²⁶, R²⁷, R²⁸, R²⁹, R³⁰, R³¹, R³², and R³³ are independently hydrogen, unsubstituted alkyl, unsubstituted heteroalkyl, unsubstituted cycloalkyl, unsubstituted heterocycloalkyl, unsubstituted aryl, or unsubstituted heteroaryl. R²⁰, R²¹, R²², R²³, R²⁴, R²⁵, R²⁶, R²⁷, R²⁸, R²⁹, R³⁰, R³¹, R³², and R³³ are independently hydrogen, unsubstituted alkyl, or unsubstituted heteroalkyl.

In some embodiments, R¹ is R³-substituted or unsubstituted alkyl, R³-substituted or unsubstituted cycloalkyl, or R³-substituted or unsubstituted aryl. In other embodiments, R¹ is R³-substituted or unsubstituted alkyl, or R³-substituted or unsubstituted cycloalkyl. In some embodiments, R¹ is R³-substituted or unsubstituted C₁-C₄ alkyl, or R³-substituted or unsubstituted C₃-C₆ cycloalkyl. In other embodiments, R¹ is R³-substituted or unsubstituted C₁-C₄ alkyl, or R³-substituted or unsubstituted cyclopentyl. In yet other embodiments, R¹ is methyl or unsubstituted C₃-C₆ branched alkyl (e.g. isopropyl, isobutyl, etc.).

In certain embodiments, R³ is R¹⁹-substituted or unsubstituted alkyl, R¹⁹-substituted or unsubstituted cycloalkyl, or R¹⁹-substituted or unsubstituted aryl. In other embodiments, R³ is R¹⁹-substituted or unsubstituted alkyl, R¹⁹-substituted or unsubstituted cycloalkyl, or R¹⁹-substituted or unsubstituted aryl. In some embodiments, R³ is R¹⁹-substituted or unsubstituted alkyl, or R¹⁹-substituted or unsubstituted cycloalkyl.

In various embodiments, R¹⁹ is unsubstituted alkyl or unsubstituted cycloalkyl. In some embodiments, R¹⁹ is unsubstituted C₁-C₄ alkyl or unsubstituted cyclopentyl.

In some embodiments, R² is R⁴-substituted aryl, or R⁴-substituted or unsubstituted heteroaryl. R² may be R⁴-substituted phenyl, R⁴-substituted or unsubstituted naphthyl, R⁴-substituted or unsubstituted pyridinyl, R⁴-substituted or unsubstituted pyrimidinyl, R⁴-substituted or unsubstituted thiophenyl, R⁴-substituted or unsubstituted furanyl, R⁴-substituted or unsubstituted indolyl, R⁴-substituted or unsubstituted benzoxadiazolyl, R⁴-substituted or unsubstituted benzodioxolyl, R⁴-substituted or unsubstituted benzodioxanyl, R⁴-substituted or unsubstituted thianaphthanyl, R⁴-substituted or unsubstituted pyrrolopyridinyl, R⁴-substituted or unsubstituted indazolyl, R⁴-substituted or unsubstituted tetrahydronaphthalenyl, R⁴-substituted or unsubstituted quinolinyl, R⁴-substituted or unsubstituted quinoxalinyl, R⁴-substituted or unsubstituted pyridopyrazinyl, R⁴-substituted or unsubstituted quinazolinonyl, R⁴-substituted or unsubstituted chromenonyl, R⁴-substituted or unsubstituted benzoisoxazolyl, R⁴-substituted or unsubstituted imidazopyridinyl, R⁴-substituted or unsubstituted benzofuranyl, R⁴-substituted or unsubstituted dihydro-benzofuranyl, R⁴-substituted or unsubstituted dihydro-benzodioxinyl, R⁴-substituted or unsubstituted benzoimidazolonyl, or R⁴-substituted or unsubstituted benzothiophenyl.

In certain embodiments, R² is R⁴-substituted phenyl, R⁴-substituted or unsubstituted pyrrolepyridinyl, R⁴-substituted or unsubstituted quinolinyl, R⁴-substituted or unsubstituted indazolyl, R⁴-substituted or unsubstituted quinolinyl, R⁴-substituted or unsubstituted indolyl, or R⁴-substituted or unsubstituted naphthyl. In some embodiments, R⁴ is halogen, —CN, —OR²⁰, —NR²²R²³. In other embodiments, R⁴ is halogen, or OR²⁰. In some embodiments, R²⁰ is hydrogen, alkyl or aryl. In various embodiments, R²⁰ is hydrogen. In some embodiments, R⁴ is —OH.

In some embodiments, R² is a moiety of one of the following structures:

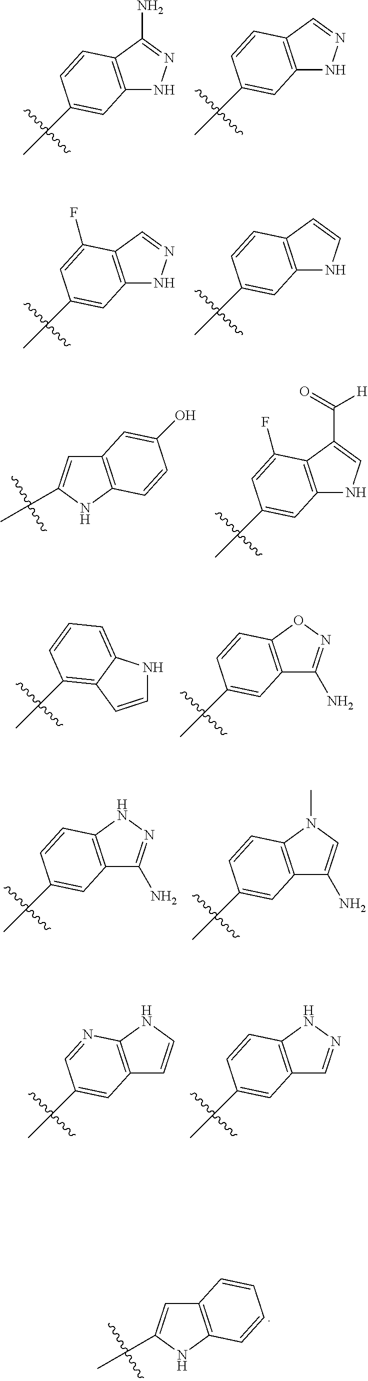

In some embodiments, R² is 5-hydroxy indol-2-yl. In some embodiments, R² is indol-2-yl.

In some embodiments, R² is a moiety of Formula IV:

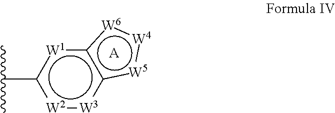

where ring A is aromatic; W¹, W², and W³ are independently CR⁴ or N; W⁴ is CH, N, or O, W⁵ is N, O, NH or NR^{a}; W⁶ is CR⁴ or N; no two adjacent ring atoms are oxygen and/or sulfur and no more than two adjacent ring atoms are nitrogen. In some embodiments, ring A comprises an oxygen ring atom and a nitrogen ring atom.

In some embodiments, W⁴ is CH, W⁵ is NH and W⁶ is N or CR⁴; W⁴ is CH, W⁵ is NR^{a} and W⁶ is N or CR⁴; W⁴ is CH or N, W⁵ is O and W⁶ is N or CR⁴; W⁴ is N, W⁵ is NH and W⁶ is CR⁴; W⁴ is O and W⁶ is N or CR⁴; or W⁴ is O, W⁵ is N and W⁶ is N or CR⁴. In some embodiments, W⁴ is N, W⁵ is O and W⁶ is CR⁴.

In some embodiments, R² is a moiety of Formula V:

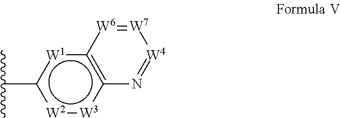

wherein W¹, W², and W³ are independently CH, CR⁴ or N; W⁴ and W⁶ are independently CH, CR⁴, or N; and W⁷ is CH or N; and wherein no more than two adjacent ring atoms are N.

In some embodiments, R¹ is R³-substituted or unsubstituted alkyl, or R³-substituted or unsubstituted heterocycloalkyl, and R² is a moiety of Formula VI:

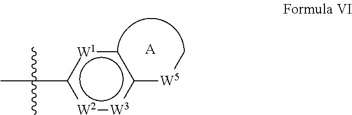

wherein ring A is a five membered aromatic ring; W¹, W², and W³ are independently CR⁴ or N; W⁵ is N, O, NH or NR⁴; and wherein R⁴ is —C(O)Oalkyl, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, ring A further comprises no more than three heteroatoms. In some embodiments, R¹ is alkyl. In other embodiments, R¹ is isopropyl. In yet other embodiments, R¹ is substituted heterocycloalkyl.

In various embodiments, R² is a moiety of the following structure:

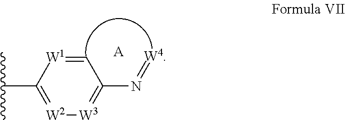

In Formula VII, W¹, W², W³, and W⁴ are independently ═CH—, ═CR⁴—, or ═N—. Each R⁴ is as defined above in the description of Formulae I and II. Ring A is a substituted or unsubstituted heteroaryl or substituted or unsubstituted heterocycloalkyl. In some embodiments, ring A is a 6 to 7 membered heterocycloalkyl or 6 to 7 membered heteroaryl. Thus, in some embodiments, ring A is partially or fully unsaturated 6- or 7-membered ring.

R²⁰ may be hydrogen or unsubstituted C₁-C₁₀ alkyl. In some embodiments, R²⁰ is hydrogen or unsubstituted C₁-C₄ alkyl. R²⁰ may also simply be hydrogen or methyl.

In some embodiments, R² is of one of the following formulae:

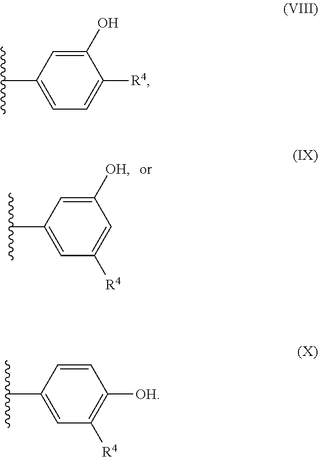

In Formulae VIII, IX and X, R⁴ is absent, halogen, unsubstituted C₁-C₄ alkyl, or —OR²⁰. The halogen may be F, Cl, or Br. In some embodiments, the halogen is F or Cl. In other embodiments, the halogen is F. In some embodiments, R²⁰ is hydrogen or unsubstituted C₁-C₄ alkyl.

In some embodiments, R² is 6-hydroxynaphthyl, unsubstituted 7-azaindole, unsubstituted indolyl, unsubstituted indazolyl, or unsubstituted quinolinyl.

In some embodiments, R² is one of the following formulae:

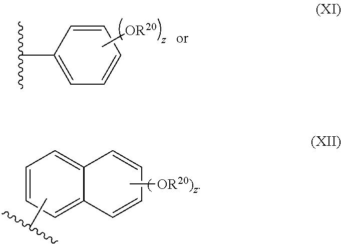

In Formulae XI and XII, R²° is as defined above. It is noted that, in accordance with the description of R² above, each R² is optionally different. The symbol z is an integer from 1 to 5 (e.g. 1 or 2). In some embodiments, R²⁰ is hydrogen or unsubstituted C₁-C₁₀ alkyl (e.g. C₁-C₅ alkyl such as methyl or ethyl).

In some embodiments, R² is a moiety of one of the following formulae:

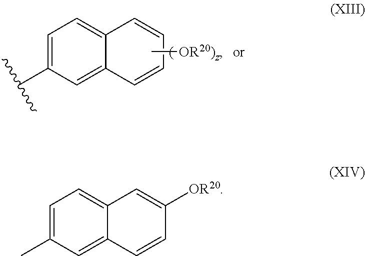

In Formulae XIII and XIV, above, R²⁰ is as defined above, for example, in the description of Formulae I, II, X, and XI above.

In some embodiments, each substituted group described above for the compounds of the present invention is substituted with at least one substituent group. More specifically, in some embodiments, each substituted alkyl, substituted heteroalkyl, substituted cycloalkyl, substituted heterocycloalkyl, substituted aryl, substituted heteroaryl, aryl(C₁-C₆)alkyl, and heteroaryl(C₁-C₆)alkyl described above is substituted with at least one substituent group. In other embodiments, at least one or all of these groups are substituted with at least one size-limited substituent group. Alternatively, at least one or all of these groups are substituted with at least one lower substituent group.

In other embodiments of the compounds described above, each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, each substituted or unsubstituted heterocycloalkyl is a substituted or unsubstituted 4 to 8 membered heterocycloalkyl.

Alternatively, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

In another embodiment, the present invention provides compound of Formula Ia or Ib for use in the methods of the invention:

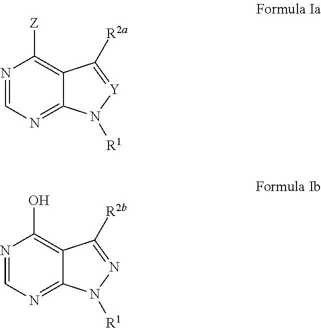

or a pharmaceutically-acceptable salt or prodrug thereof; wherein:

Y is N or CR₅;

Z is NR₃R₄, halo, H, OH, alkyl, alkyloxy, or haloalkyl;

R¹ is C₁-C₆ alkyl or C₄-C₇ cycloalkyl, wherein said alkyl or said cycloalkyl is optionally substituted with mono- or di-alkoxy, mono- or di-halophenyl, mono- or di-(C₁₋₄)alkoxy phenyl, mono- or di-(C₁₋₄)alkyl phenyl, perhalo(C₁₋₄)alkyl phenyl, carboxyl, tert-butyl carboxyl, phosphoryl, (C₁₋₆)alkyl, (C₄₋₇)cycloalkyl, indolyl, isoindolyl, pyridyl, naphthyl, pyrrolyl, imidazolyl, pyrazolyl, pyridyl, pyrimidinyl, pyrazinyl, pyridazinyl, furyl, thienyl, or alkylmorpholino;

R^{2a} is indolyl, thiazolyl, benzyl, biphenylyl, thiophenyl, pyrrolyl, or phenyl, wherein said phenyl is substituted with at least one of OH, —NR₃R₄, —C(═O)NR₆R₇, —CN, NO_{2—}C(═O)OH, —C(═O)O-alkyl, (C₁₋C₄)alkyl, halo, haloalkyl or haloaryl; and wherein said indolyl, thiazolyl, benzyl, biphenylyl, thiophenyl, or pyrrolyl is optionally substituted with OH, —NR₃R₄, —C(═O)NR₆R₇, —CN, NO₂, —C(═O)O—R₃, (C₁₋C₄)alkyl, halo, haloalkyl or haloaryl;

R^{2b} is indolyl, thiazolyl, benzyl, biphenylyl, thiophenyl, pyrrolyl, or phenyl wherein said indoyl, thiazolyl, benzyl, biphenylyl, thiophenyl, pyrrolyl, phenyl is optionally substituted with —OH, —NR₃R₄, —C(═O)NR₆R₇, —CN, NO₂, —C(═O)O—R₃, (C₁-C₄)alkyl, halo, haloalkyl, or haloaryl;

R₃ and R₄ are independently H, C₁-C₆ alkyl, t-Boc, morpholino(C₁-C₄)alkyl, carboxy(C₁-C₃)alkyl, (C₁-C₄)alkoxycarbonyl(C₁-C₃)alkyl, aryl, heteroaryl, aryloxy, heterocycle, cycloalkyl, alkenyl with the proviso that the double bond of the alkenyl is not present at the carbon atom that is directly linked to N, alkynyl with the proviso that the triple bond of the alkynyl is not present at the carbon atom that is directly linked to N, perfluoroalkyl, —S(O)₂alkyl, —S(O)₂aryl, —(C═O)heteroaryl, —(C═O)aryl, —(C═O)(C₁-C₆)alkyl, —(C═O)cycloalkyl, —(C═O)heterocycle, alkyl-heterocycle, aralkyl, arylalkenyl, —CON R₆R₇, —SO₂R₆R₇, arylalkoxyalkyl, arylalkylalkoxy, heteroarylalkylalkoxy, aryloxyalkyl, heteroaryloxyalkyl, aryloxyaryl, aryloxyheteroaryl, alkylaryloxyaryl, alkylaryloxyheteroaryl, alkylaryloxyalkyamine, alkoxycarbonyl, aryloxycarbonyl, or heteroaryloxycarbonyl;

R₅ are independently H, —OH, halo, optionally monosubstituted (C₁-C₆)alkyl, optionally monosubstituted (C₁-C₄)alkoxycarbonyl, optionally monosubstituted (C₁-C₄)alkanoyl, carbamoyl, optionally monosubstituted (C₁-C₄)alkyl carbamoyl, phenyl, halophenyl, optionally monosubstituted (C₁-C₄)alkylphenyl, optionally monosubstituted (C₁-C₄)alkoxyphenyl, or optionally monosubstituted perhalo(C₁-C₄)alkylphenyl, wherein said optional substitution is (C₁-C₄)alkyl, OH, or halogen;

R₆ and R₇ are independently H, alkyl, aryl, heteroaryl, alkylaryl, arylalkyl, heteroarylalkyl, or alkylheteroaryl;

In various embodiments, Y is N.

In one embodiment, R^{2a} or R^{2b} is phenyl substituted with mono, di or tri-OH. In a further embodiment, the phenyl is further substituted with a halo. In a further detailed embodiment, the halo is F. In another embodiment, R^{2a} or R^{2b} is indolyl. In some embodiments, R^{2a} or R^{2b} is 2-indolyl.

In some embodiments, R¹ is 2-methyl-propane. In a detailed embodiment, R₃ and R₄ are H. In a detailed embodiment, R₅ is H. In a detailed embodiment, R₆ is H and R₇ is methyl.

In certain embodiments, R^{2a} is, independently, phenyl substituted at a meta position with —CH₃, tert-butyl, —CF₃ or halo. In a detailed embodiment, R^{2a} is, independently, phenyl substituted at a meta position with halo, alkyl, haloalkyl, haloaryl, aryl, O-alkyl, CN, NO₂, CO—O—R₃, CO—N(R₃)₂. In a detailed embodiment, Z is F, Br Cl, or I.

In some embodiments, the compounds of Formula Ia or Formula Ib include: 3-(4-amino-7-isopropyl-7H-pyrrolo[2,3-d]pyrimidin-5-yl)-phenol; 3-(7-isopropyl-4-methylamino-7H-pyrrolo[2,3-d]pyrimidin-5-yl)-phenol; [5-(3-amino-phenyl)-7-isopropyl-7H-pyrrolo[2,3-d]pyrimidin-4-yl]-methyl-a-mine; 3-(4-benzylamino-7-isopropyl-7H-pyrrolo[2,3-d]pyrimidin-5-yl)-phenol- ; 3-(4-dibenzylamino-7-isopropyl-7H-pyrrolo[2,3-d]pyrimidin-5-yl)-phenol; 3-[5-(3-hydroxy-phenyl)-4-methylamino-pyrrolo[2,3-d]pyrinidin-7-yl]-propi-onic acid tert-butyl ester; 3-[5-(3-hydroxy-phenyl)-4-methylamino-pyrrolo[2,3-d]pyrimidin-7-yl]-propi-onic acid; 3-bromo-5-(7-isopropyl-4-methylamino-7H-pyrrolo[2,3-d]pyrimidin- -5-yl)-phenol; 3-(7-isopropyl-4-methylamino-7H-pyrrolo[2,3-d]pyrimidin-5-yl)-5-methyl-ph-enol; 3-tert-Butyl-5-(7-isopropyl-4-methylamino-7H-pyrrolo[2,3-d]pyrimidin-5-yl)-phenol; 3-(7-Isopropyl-4-methylamino-7H-pyrrolo[2,3-d]pyrimidin-5-yl)-5-trifluoro-methyl-phenol; 3-bromo-5-(4-chloro-7-isopropyl-7H-pyrrolo[2,3-d]pyrimidin-5-yl)-phenol; 3-(4-chloro-7-isopropyl-7H-pyrrolo[2,3-d]pyrimidin-5-yl)-5-methyl-phenol; 3-tert-butyl-5-(4-chloro-7-isopropyl-7H-pyrrolo[2,3-d]pyrimidin-5-yl)-phenol; 3-(4-Chloro-7-isopropyl-7H-pyrrolo[2,3-d]pyrimidin-5-yl)-5-trifluoromethyl-phenol or a pharmaceutically-acceptable salt or prodrug thereof.

In another aspect, the invention provides a PI3-kinase antagonists which is a compound of Formula XV, or a pharmaceutically acceptable salt thereof:

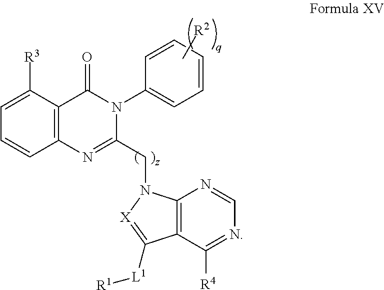

In Formula XV above, q is an integer of 0, 1, 2, 3, 4, or 5; z is an integer of 0, 1, 2, 3, 4, 5, 6, 7, 8, 9, or 10; and X is ═CH— or ═N—. L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹ and R² are independently halogen, —CN, —OR⁵, —S(O)ₙR⁶, —NR⁷R⁸, —C(O)R⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, where n is an integer from 0 to 2. R³ and R⁴ are independently hydrogen, halogen, —CN, —OR⁵, —S(O)ₙR⁶, —NR⁷R⁸, —C(O)R⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, where n is an integer from 0 to 2.

R⁵ is independently hydrogen, —C(O)R¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶ is independently hydrogen, —NR¹¹R¹², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. Where n is 1 or 2, R⁶ is other than hydrogen.

R⁷ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁸ is independently hydrogen, —S(O)ₙR¹³, —C(O)R¹⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R⁹ is independently —NR¹⁵R¹⁶, hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹⁰ is independently hydrogen, —NR¹⁷R¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹⁴ is independently hydrogen, —NR¹⁹R^{20,} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

R¹¹, R¹², R¹³, R¹⁵, R¹⁶, R¹⁷, R¹⁸, R¹⁹, and R²⁰ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, R¹ is halogen, substituted or unsubstituted halo(C₁-C₆)alkyl, substituted or unsubstituted aryl, substituted or unsubstituted heteroaryl, substituted or unsubstituted aryl(C₁-C₆)alkyl, or substituted or unsubstituted heteroaryl(C₁-C₆)alkyl. In other embodiments, R¹ is halogen, substituted or unsubstituted phenyl, substituted or unsubstituted furanyl, substituted or unsubstituted pyrrolyl, substituted or unsubstituted thiophenyl, or substituted or unsubstituted benzothiophenyl, substituted or unsubstituted indolyl, substituted or unsubstituted quinolinyl, substituted or unsubstituted pyridinyl, substituted or unsubstituted 1H-pyrrolo[2,3-c]pyridinyl, substituted or unsubstituted 1H-pyrrolo[2,3-b]pyridinyl, substituted or unsubstituted thiazolyl, substituted or unsubstituted imidazolyl, substituted or unsubstituted oxazolyl, substituted or unsubstituted isoxazolyl, substituted or unsubstituted pyrazolyl, substituted or unsubstituted isothiazolyl, substituted or unsubstituted cylcohexyl, substituted or unsubstituted morpholino, substituted or unsubstituted piperidinyl, or substituted or unsubstituted tetrahydropyridinyl.

In other embodiments, R¹ is phenyl, furanyl, pyrrolyl, thiophenyl, or benzothiophenyl, each of which are optionally substituted with one or more R²¹ substituent(s). R²¹ is independently (1) or (2) as defined in this paragraph. In various embodiments, R²¹ is halogen, —CN, —OR²², —C(O)R²³, —NR²⁴R²⁵, —S(O)_{w}NR²⁶R²⁷, or —S(O)_{w}R²⁸. The symbol w is an integer form 0 to 2. R²², R²³, R²⁴, R²⁵, R²⁶, R²⁷, and R²⁸ are independently hydrogen, alkyl, heteroalkyl, cycloalkyl, heterocycloalkyl, aryl, heteroaryl, cycloalkyl-alkyl, heterocycloalkyl-alkyl, arylalkyl, or heteroarylalkyl, optionally substituted with unsubstituted alkyl, unsubstituted heteroalkyl, unsubstituted cycloalkyl, unsubstituted heterocycloalkyl, unsubstituted aryl, unsubstituted heteroaryl, unsubstituted cycloalkyl-alkyl, unsubstituted heterocycloalkyl-alkyl, unsubstituted arylalkyl, or unsubstituted heteroarylalkyl. In other embodiments, R²¹ is (C₁-C₁₀)alkyl, 2 to 10 membered heteroalkyl, C₃-C₈ cycloalkyl, 3 to 8 membered heterocycloalkyl, aryl or heteroaryl optionally substituted with halogen, —OH, —CN, —NH₂, unsubstituted alkyl, unsubstituted heteroalkyl, unsubstituted cycloalkyl, unsubstituted heterocycloalkyl, unsubstituted aryl, unsubstituted heteroaryl, unsubstituted cycloalkyl-alkyl, unsubstituted heterocycloalkyl-alkyl, unsubstituted arylalkyl, or unsubstituted heteroarylalkyl.

In some embodiments, R¹ is phenyl substituted at the meta and para positions, or substituted at the meta and meta positions. That is, R¹ is a 4,5-substituted phenyl or a 3,5-substituted phenyl. In some related embodiments, the 4,5-substituted phenyl or 3,5-substituted phenyl is substituted, independently, with R²¹ (as defined in the previous paragraph). In some embodiments, R²¹ is halogen or —OR²². In other embodiments, R²¹ is fluorine and in various embodiments, R²² is hydrogen or unsubstituted C₁-C₄ alkyl (e.g. methyl). In other embodiments, R¹ is phenyl substituted para position (i.e. a 4-substituted phenyl).

In some embodiments, L¹ is substituted or unsubstituted alkylene (e.g. a substituted or unsubstituted alkynylene. In other embodiments, L¹ is substituted or unsubstituted methylene, substituted or unsubstituted ethylene, substituted or unsubstituted propylene, substituted or unsubstituted butylenes, substituted or unsubstituted ethynylene, or substituted or unsubstituted prop-2-ynylene. In some related embodiments, R¹ is —CN, —OR⁵, NR⁷R⁸, R²¹-substituted or unsubstituted cycloalkyl, R²¹-substituted or unsubstituted aryl, R²¹-substituted or unsubstituted heteroaryl, R²¹-substituted or unsubstituted C₁-C₄ alkyl. R²¹ may be halogen, —OR²², —NR²⁴R²⁵, or unsubstituted C₁-C₄ alkyl. R⁵, R⁷, R⁸, R²², R²⁴ and R²⁵ are independently hydrogen or unsubstituted C₁-C₄ alkyl (e.g. methyl).

In some embodiments, R² is halogen, —OH, —CN, —NH₂, unsubstituted alkyl, unsubstituted heteroalkyl, unsubstituted cycloalkyl, unsubstituted heterocycloalkyl, unsubstituted aryl, unsubstituted heteroaryl, unsubstituted cycloalkyl-alkyl, unsubstituted heterocycloalkyl-alkyl, unsubstituted arylalkyl, or unsubstituted heteroarylalkyl. In some embodiments, R² is halogen or unsubstituted alkyl. In some embodiments, R² is fluorine or unsubstituted C₁-C₄ alkyl (e.g. methyl).

In various embodiments, R³ is halogen, —OH, —CN, —NH₂, unsubstituted alkyl, unsubstituted heteroalkyl, unsubstituted cycloalkyl, unsubstituted heterocycloalkyl, unsubstituted aryl, unsubstituted heteroaryl, unsubstituted cycloalkyl-alkyl, unsubstituted heterocycloalkyl-alkyl, unsubstituted arylalkyl, or unsubstituted heteroarylalkyl. In other embodiments, R³ is unsubstituted C₁-C₄ alkyl (e.g. methyl).

In some embodiments, R⁴ is halogen, —OH, —CN, —NH₂, unsubstituted alkyl, unsubstituted heteroalkyl, unsubstituted cycloalkyl, unsubstituted heterocycloalkyl, unsubstituted aryl, unsubstituted heteroaryl, unsubstituted cycloalkyl-alkyl, unsubstituted heterocycloalkyl-alkyl, unsubstituted arylalkyl, or unsubstituted heteroarylalkyl.

In some embodiments, R² and R³ are independently unsubstituted C₁-C₄ alkyl, R⁴ is NH₂, q is 1, and z is 1.

In some embodiments, each substituted group described above in the compound of Formula XV is substituted with at least one substituent group. More specifically, in some embodiments, each substituted alkyl, substituted heteroalkyl, substituted cycloalkyl, substituted heterocycloalkyl, substituted aryl, substituted heteroaryl, substituted cycloalkyl-alkyl, substituted heterocycloalkyl-alkyl, substituted arylalkyl, and/or substituted heteroarylalkyl, described above in the compounds of Formula XV is substituted with at least one substituent group. In other embodiments, at least one or all of these groups are substituted with at least one size-limited substituent group. Alternatively, at least one or all of these groups are substituted with at least one lower substituent group.

In other embodiments of the compounds of Formula XV, each substituted or unsubstituted alkyl is a substituted or unsubstituted C₁-C₂₀ alkyl, including those alkyl groups forming part of a cycloalkyl-alkyl (i.e. a cycloalkyl-(C₁-C₂₀)alkyl), heterocycloalkyl-alkyl (i.e. a heterocycloalkyl-(C₁-C₂₀)alkyl), arylalkyl (i.e. an aryl-(C₁-C₂₀)alkyl), or substituted heteroarylalkyl (i.e. a heteroaryl-(C₁-C₂₀)alkyl). Each substituted or unsubstituted heteroalkyl is a substituted or unsubstituted 2 to 20 membered heteroalkyl. Each substituted or unsubstituted cycloalkyl is a substituted or unsubstituted C₄-C₈ cycloalkyl, including those cycloalkyl groups forming part of a cycloalkyl-alkyl (i.e. a C₄-C₈ cycloalkyl-alkyl, or a C₄-C₈ cycloalkyl-(C₁-C₂₀)alkyl). Each substituted or unsubstituted heterocycloalkyl is a substituted or unsubstituted 4 to 8 membered heterocycloalkyl, including those heterocycloalkyl groups forming part of a heterocycloalkyl-alkyl (i.e. a 4 to 8 membered heterocycloalkyl-alkyl, or a 4 to 8 membered heterocycloalkyl-(C₁-C₂₀)alkyl).

Alternatively, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, each substituted or unsubstituted heterocycloalkyl is a substituted or unsubstituted 5 to 7 membered heterocycloalkyl, including cycloalkyl-alkyl groups, heterocycloalkyl-alkyl groups, heteroarylalkyl groups, and arylalkyl groups, as described in the preceding paragraph.

In some embodiments of Formula XV, R¹ is unsubstituted or substituted aryl. In some embodiments of Formula XV, R¹ is aryl which is substituted at the meta and para positions, or substituted at the meta and meta positions. In some embodiments, the substituents independently are (1) halogen, —CN, —OR²², —C(O)R²³, —NR²⁴R²⁵, —S(O)_{w}NR²⁶R²⁷, or —S(O)_{w}R²⁸, wherein w is an integer from 0 to 2, and R²², R²³, R²⁴, R²⁵, R²⁶, R²⁷, and R²⁸ are independently hydrogen, alkyl, heteroalkyl, cycloalkyl, heterocycloalkyl, aryl, heteroaryl, cycloalkyl-alkyl, heterocycloalkyl-alkyl, arylalkyl, or heteroarylalkyl, optionally substituted with unsubstituted alkyl, unsubstituted heteroalkyl, unsubstituted cycloalkyl, unsubstituted heterocycloalkyl, unsubstituted aryl, unsubstituted heteroaryl, unsubstituted cycloalkyl-alkyl, unsubstituted heterocycloalkyl-alkyl, unsubstituted arylalkyl, or unsubstituted heteroarylalkyl; or (2) (C₁-C₁₀)alkyl, 2 to 10 membered heteroalkyl, C₃-C₈ cycloalkyl, 3 to 8 membered heterocycloalkyl, aryl or heteroaryl optionally substituted with halogen, —OH, —CN, —NH₂, unsubstituted alkyl, unsubstituted heteroalkyl, unsubstituted cycloalkyl, unsubstituted heterocycloalkyl, unsubstituted aryl, unsubstituted heteroaryl, unsubstituted cycloalkyl-alkyl, unsubstituted heterocycloalkyl-alkyl, unsubstituted arylalkyl, or unsubstituted heteroarylalkyl.

In some embodiments of Formula XV, L¹ is a covalent bond. In some embodiments of Formula XV, L¹ is substituted alkylene, and is substituted by unsubstituted or substituted cycloalkyl, unsubstituted or substituted aryl, —OR′, —NR′—C(O)NR″R′″, —NR′R″, —CN, wherein R′, R″, R′″ and R″″ each independently are hydrogen or substituted or unsubstituted alkyl.

In some embodiments of Formula XV, R² is unsubstituted or substituted alkyl. In various embodiments of Formula XV, R³ is unsubstituted or substituted alkyl. In some embodiments of Formula XV, R⁴ is NR⁷R⁸. In other embodiments of Formula XV, R⁴ is NH₂.

### Exemplary Compounds and In-Vitro Assay Results

Exemplary compounds of Formula I include, but are not limited to, any or all of the compounds listed in Table 1 below. Unless otherwise specified in an entry in the table, X═N.

TABLE 1

| Cpd | R¹ | R² |
|---|---|---|
| KS167 | —H | 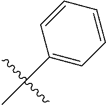 |
| ZK141 | —H | 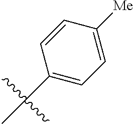 |
| KS84 | 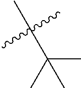 | 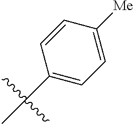 |
| ZK127 | —H | 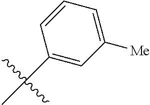 |
| ZK134 | —H | 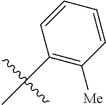 |
| ZK132 | —H | 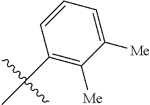 |
| ZK125 | —H | 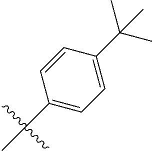 |
| BA56 | 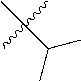 | 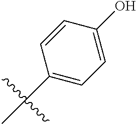 |
| ZK138 | —H | 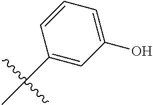 |
| KS287 | 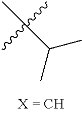 | 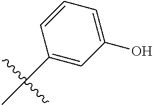 |
| KS288 | 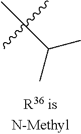 | 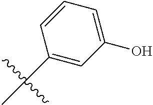 |
| KS284 | 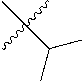 | 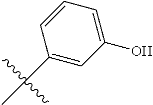 |
| BA60 | 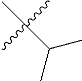 | 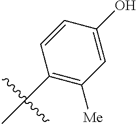 |
| ZK318 | —H | 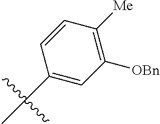 |
| ZK320 | —H | 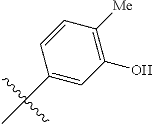 |
| ZK333 | —Me | 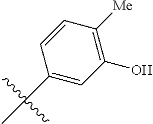 |
| ZK323 | 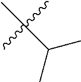 | 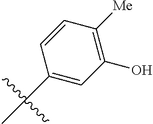 |
| ZK327 | 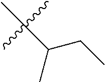 | 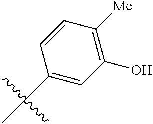 |
| BA77d | 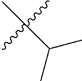 | 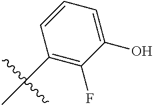 |
| BA78d | 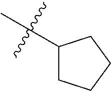 | 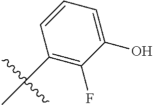 |
| BA22 | 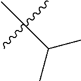 | 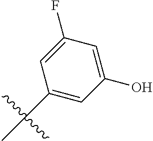 |
| BA79d | 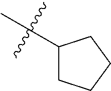 | 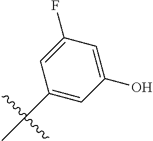 |
| BA85 | 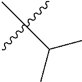 | 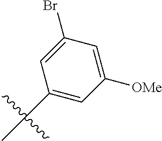 |
| BA87 | 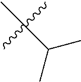 | 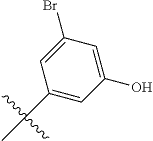 |
| ZK502 | 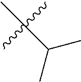 | 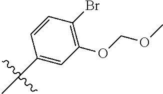 |
| ZK489 | —Me | 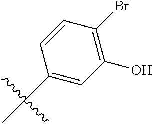 |
| ZK487 | 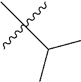 | 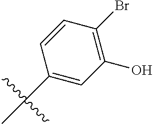 |
| Zk491 | 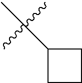 | 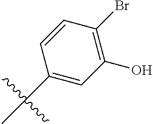 |
| Zk493 | 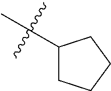 | 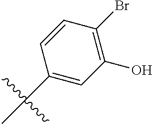 |
| BA62d | 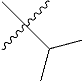 | 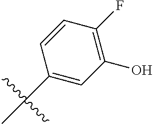 |
| ZK450 | 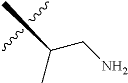 | 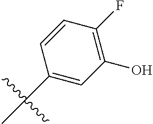 |
| ZK454 | 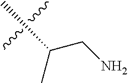 | 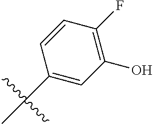 |
| ZK469 | 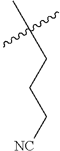 | 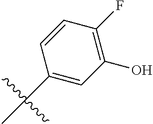 |
| ZK471 | 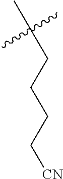 | 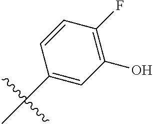 |
| ZK461 | 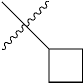 | 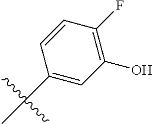 |
| ZK413 | 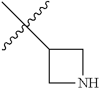 | 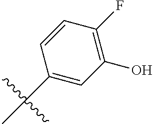 |
| ZK379 | 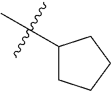 | 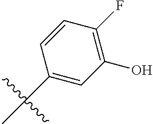 |
| ZK421 | 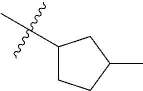 | 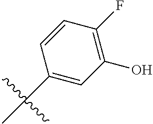 |
| ZK403 | 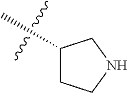 | 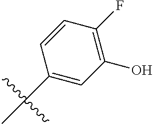 |
| ZK405 | 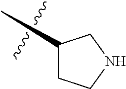 | 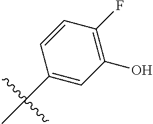 |
| ZK432 | 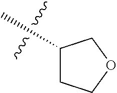 | 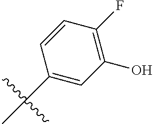 |
| ZK434 | 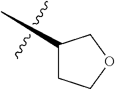 | 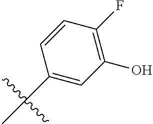 |
| ZK465 | 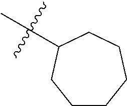 | 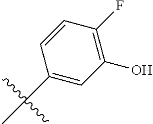 |
| ZK377 | 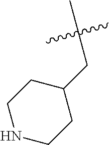 | 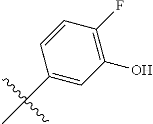 |
| ZK399 | 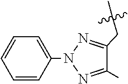 | 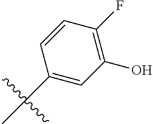 |
| ZK401 | 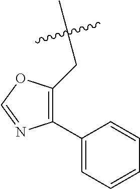 | 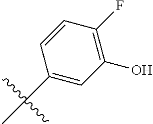 |
| BA62 | 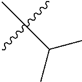 | 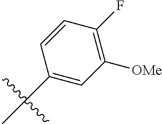 |
| ZK358 | 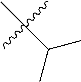 | 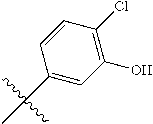 |
| ZK452 | 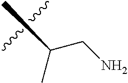 | 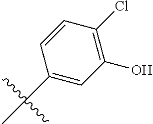 |
| ZK456 | 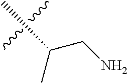 | 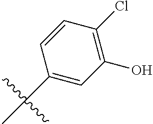 |
| ZK463 | 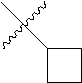 | 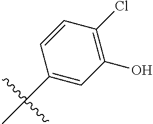 |
| ZK371 | 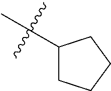 | 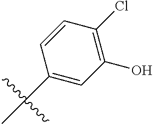 |
| ZK409 | 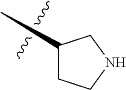 | 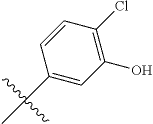 |
| ZK428 | 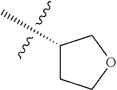 | 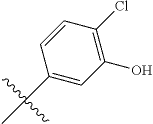 |
| ZK430 | 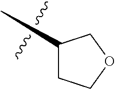 | 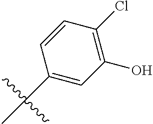 |
| ZK387 | 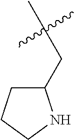 | 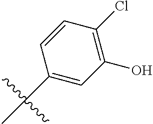 |
| ZK389 | 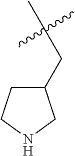 | 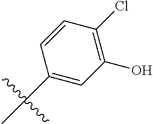 |
| ZK369 | 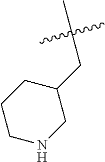 | 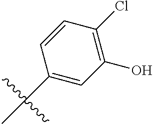 |
| ZK385 | 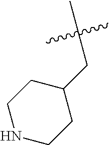 | 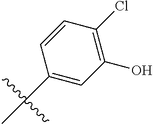 |
| ZK391 | 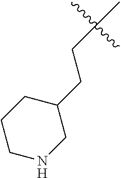 | 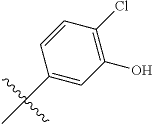 |
| BA155_2 | 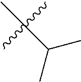 | 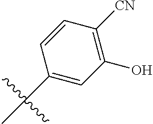 |
| BA157_2 | 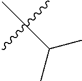 | 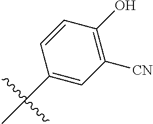 |
| BA59 | 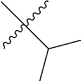 | 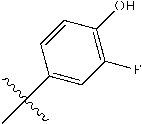 |
| BA63 | 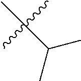 | 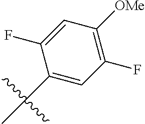 |
| BA93 | 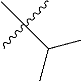 | 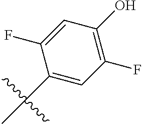 |
| BA49 | 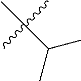 | 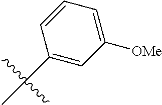 |
| BA15 | 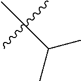 | 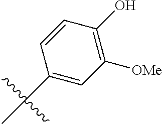 |
| ZK321 | —H | 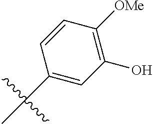 |
| ZK337 | —Me | 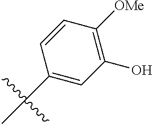 |
| ZK347 | 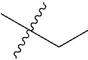 | 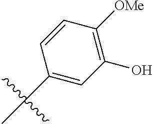 |
| ZK325 | 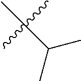 | 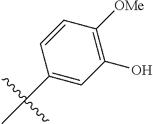 |
| ZK349 | 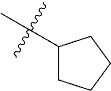 | 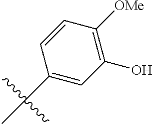 |
| ZK423 | 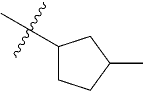 | 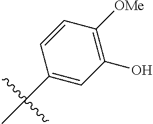 |
| ZK411 | 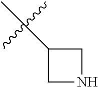 | 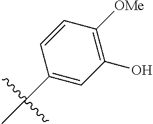 |
| ZK407 | 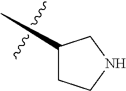 | 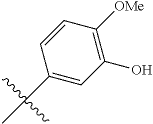 |
| BA98 | 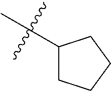 | 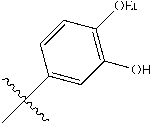 |
| BA23 | 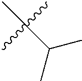 | 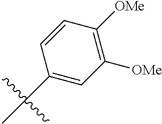 |
| ZK485 | 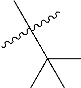 | 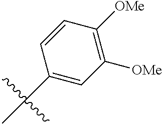 |
| ZK495 | 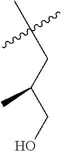 | 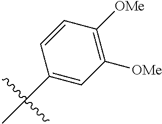 |
| ZK496 | 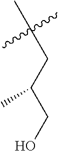 | 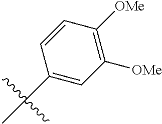 |
| ZK494 | 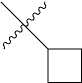 | 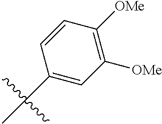 |
| BA90 | 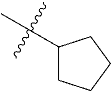 | 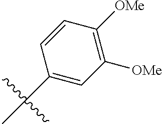 |
| ZK341 | —H | 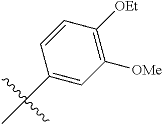 |
| ZK343 | 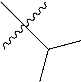 | 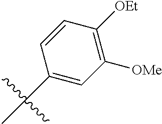 |
| ZK361 | 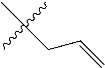 | 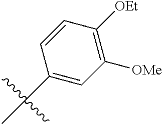 |
| ZK359 | 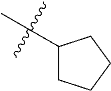 | 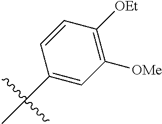 |
| ZK362 | 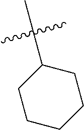 | 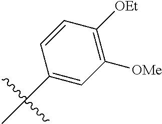 |
| BA64 | 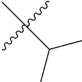 | 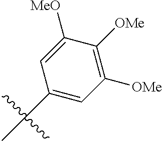 |
| BA65 | 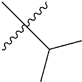 | 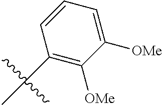 |
| ZK305 | 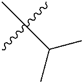 | 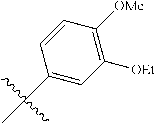 |
| ZK306 | 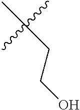 | 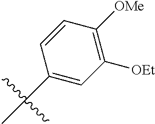 |
| BA66 | 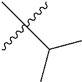 | 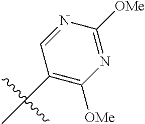 |
| BA48 | 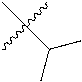 | 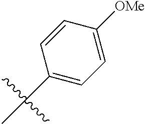 |
| ZK133 | —H | 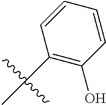 |
| BA20dd | 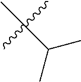 | 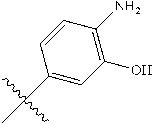 |
| BA20d | 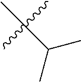 | 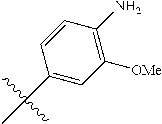 |
| BA20 | 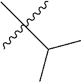 | 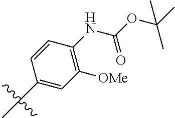 |
| BA99 | 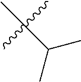 | 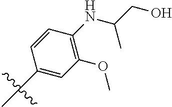 |
| BA81dd | 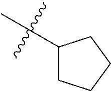 | 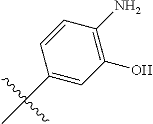 |
| BA81d | 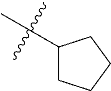 | 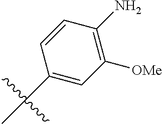 |
| ZK137 | —H | 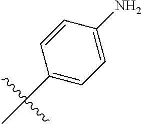 |
| ZK135 | —H | 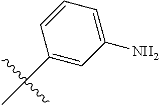 |
| ZK130 | —H | 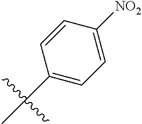 |
| ZK128 | —H | 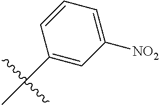 |
| BA26 | 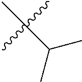 | 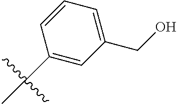 |
| BA46 | 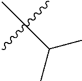 | 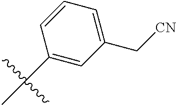 |
| BA45 | 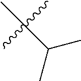 | 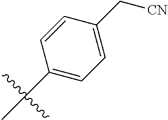 |
| BA39 | 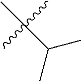 | 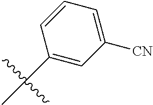 |
| BA150 | 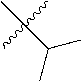 | 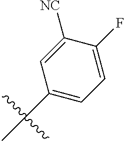 |
| BA151 | 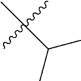 | 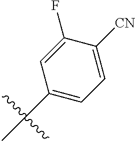 |
| BA21 | 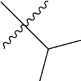 | 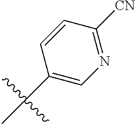 |
| BA52 | 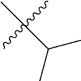 | 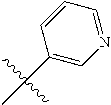 |
| BA53 | 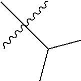 | 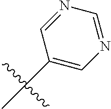 |
| BA31 | 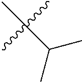 | 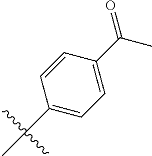 |
| BA152 | 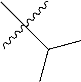 | 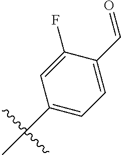 |
| BA14 | 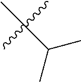 | 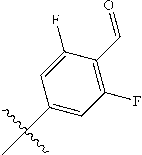 |
| BA32 | 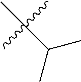 | 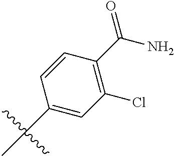 |
| BA55 | 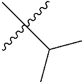 | 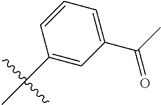 |
| BA35 | 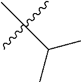 | 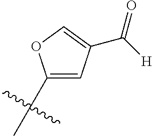 |
| BA34 | 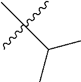 | 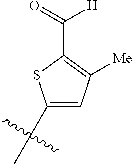 |
| BA38 | 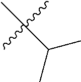 | 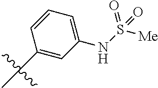 |
| BA40 | 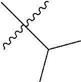 | 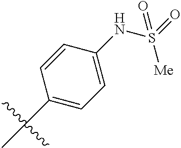 |
| BA41 | 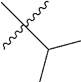 | 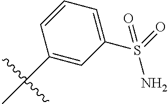 |
| BA14 | 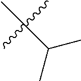 | 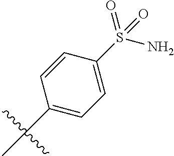 |
| BA12 | 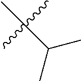 | —I |
| BA30 | 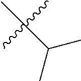 | 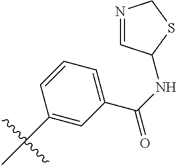 |
| ZK149 | —H | 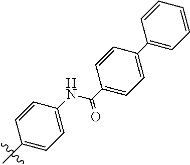 |
| ZK126 | —H | 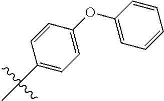 |
| ZK143 | —H | 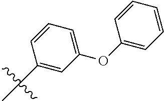 |
| ZK150 | —Me X = CH | 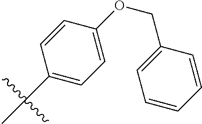 |
| ZK136 | —H | 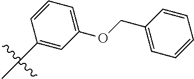 |
| ZK131 | —H | 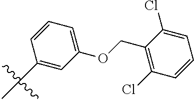 |
| ZK151 | —Me X = CH | 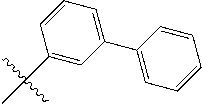 |
| ZK142 | —H | 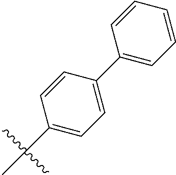 |
| ZK139 | —H | 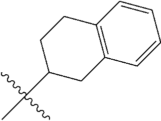 |
| KS207 | —H | 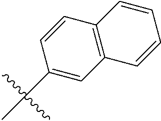 |
| KS208 | —Me | 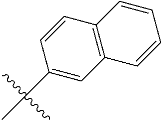 |
| ZK102 | —Me (R³⁶ is Cl) | 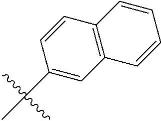 |
| ZK157 | 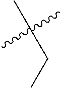 | 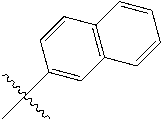 |
| ZK159 | 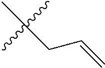 | 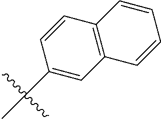 |
| ZK156 | 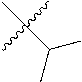 | 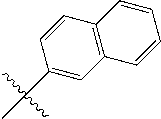 |
| KS63 | 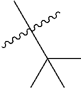 | 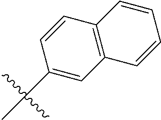 |
| ZK158 | 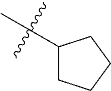 | 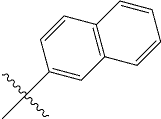 |
| ZK147 | 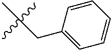 | 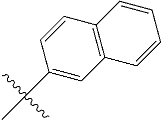 |
| ZK155 | 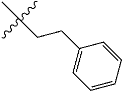 | 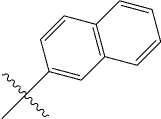 |
| ZK162 | 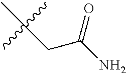 | 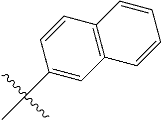 |
| ZK165 | 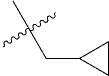 | 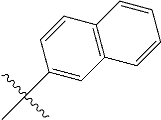 |
| ZK161 | 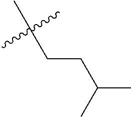 | 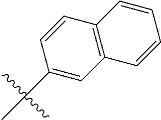 |
| ZK167 | 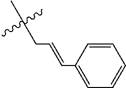 | 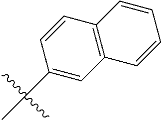 |
| ZK168 | 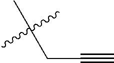 | 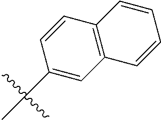 |
| BA116 | —Me | 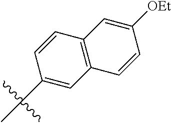 |
| BA17 | 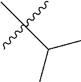 | 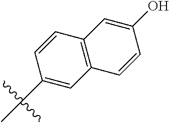 |
| BA134 | 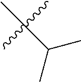 | 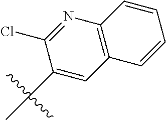 |
| BA105 | 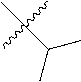 | 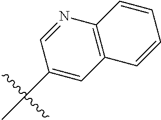 |
| BA122 | 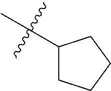 | 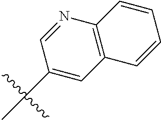 |
| BA111 | —Me | 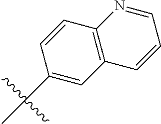 |
| BA102 | 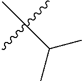 | 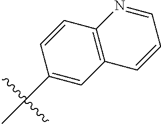 |
| BA112 | 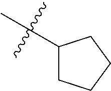 | 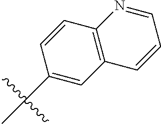 |
| BA118 | 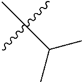 | 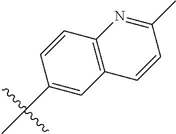 |
| BA130 | 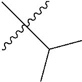 | 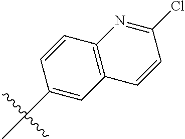 |
| BA132 | 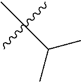 | 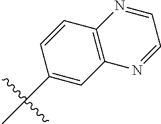 |
| BA139 | 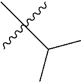 | 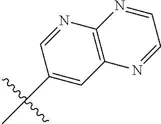 |
| BA158 | 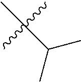 | 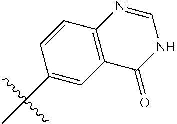 |
| BA140 | 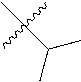 | 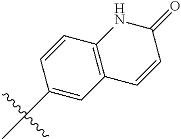 |
| BA141 | 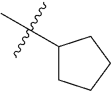 | 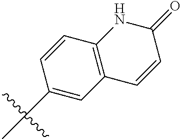 |
| BA146 | 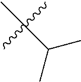 | 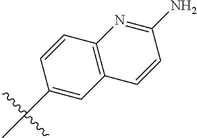 |
| BA142 | 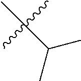 | 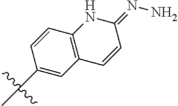 |
| BA145 | 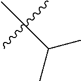 | 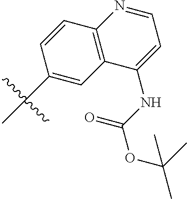 |
| BA147 | 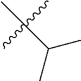 | 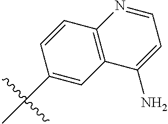 |
| BA148 | —Me | 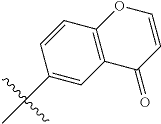 |
| BA143 | 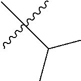 | 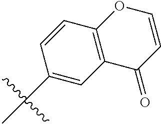 |
| BA144 | 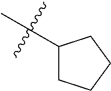 | 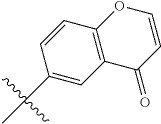 |
| BA129 | 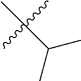 | 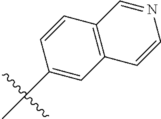 |
| BA131 | 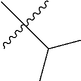 | 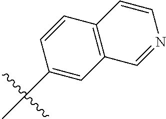 |
| BA133 | 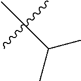 | 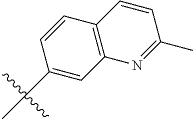 |
| BA120 | —Me | 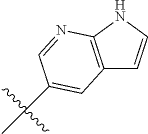 |
| BA108 | 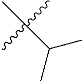 | 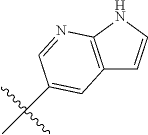 |
| BA121 | 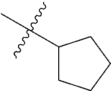 | 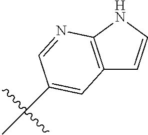 |
| BA89 | 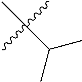 | 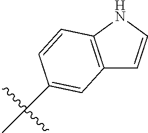 |
| BA94 | 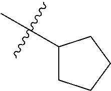 | 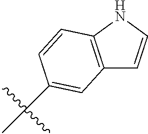 |
| BA135 | 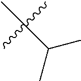 | 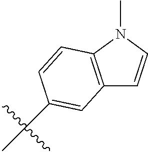 |
| BA137 | 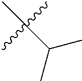 | 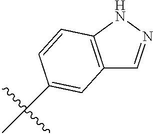 |
| BA138 | 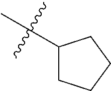 | 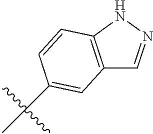 |
| BA160 | 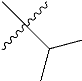 | 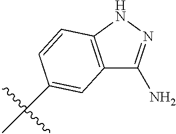 |
| BA157_3 | 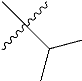 | 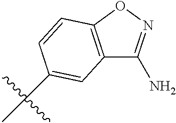 |
| BA154 | 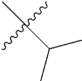 | 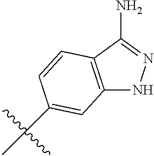 |
| BA110 | 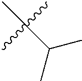 | 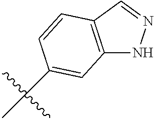 |
| BA115 | 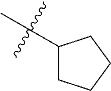 | 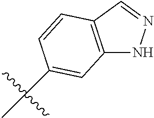 |
| BA159 | 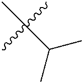 | 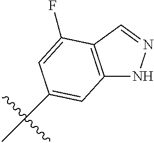 |
| BA119 | —Me | 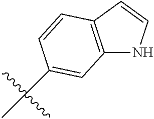 |
| BA107 | 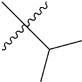 | 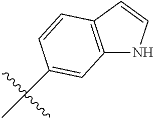 |
| BA124 | 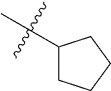 | 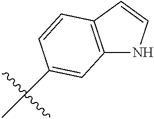 |
| BA161 | 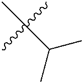 | 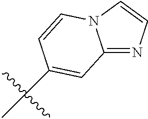 |
| BA162 | 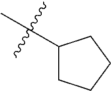 | 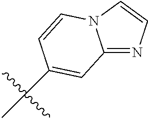 |
| BA24dd | 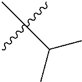 | 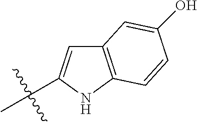 |
| BA43 | 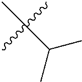 | 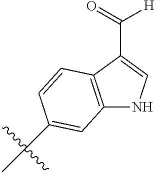 |
| BA91 | 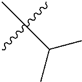 | 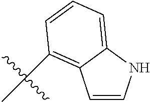 |
| BA92 | 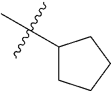 | 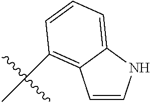 |
| BA86 | 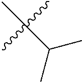 | 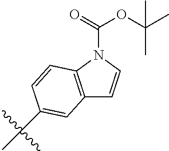 |
| BA88 | 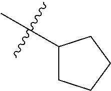 | 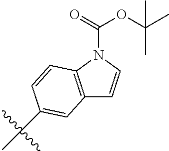 |
| BA96 | 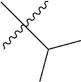 | 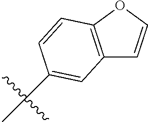 |
| BA97 | 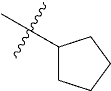 | 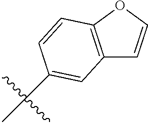 |
| BA44 | 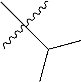 | 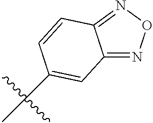 |
| BA156 | 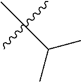 | 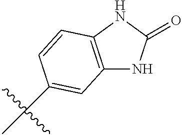 |
| BA95 | 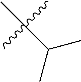 | 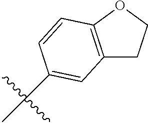 |
| ZK129 | —Me X = CH | 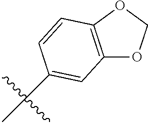 |
| BA54 | 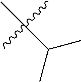 | 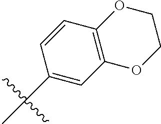 |
| ZK152 | —Me X = CH | 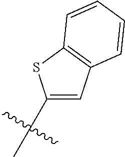 |
| BA42 | 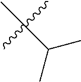 | 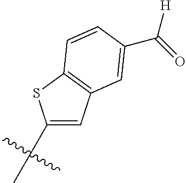 |
| T100 | 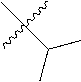 | 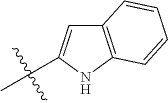 |

TABLE 2
IC50 values for the Compounds of Table 1 in-vitro assays against p110α, p110β, p110δ, p110γ, and DPK.

| Cpd | p110α | p110β | p110δ | p110γ | DPK | Cpd | p110α | p110β | p110δ | p110γ | DPK |
|---|---|---|---|---|---|---|---|---|---|---|---|
| KS167 | ++ | + | ++ | ++ | ++ | BA46 | ++ | ++ | +++ | ++ | +++ |
| ZK141 | ++ | ++ | ++ | ++ | +++ | BA45 | ++ | + | ++ | ++ | ++ |
| KS84 | ++ | + | ++ | + | +++ | BA39 | ++ | | +++ | ++ | ++ |
| ZK127 | ++ | ++ | ++ | ++ | +++ | BA150 | ++ | + | ++ | ++ | ++ |
| ZK134 | ++ | + | ++ | ++ | +++ | BA151 | ++ | + | ++ | ++ | ++ |
| ZK132 | ++ | ++ | ++ | ++ | +++ | BA21 | ++ | ++ | ++ | | ++ |
| ZK125 | ++ | + | ++ | ++ | +++ | BA52 | ++ | ++ | ++ | ++ | ++ |
| BA56 | +++ | +++ | +++ | +++ | +++ | BA53 | ++ | + | + | + | ++ |
| ZK138 | ++ | ++ | ++ | ++ | +++ | BA31 | ++ | ++ | +++ | +++ | ++ |
| KS287 | ++ | ++ | ++ | ++ | ++ | BA152 | ++ | ++ | +++ | ++ | +++ |
| KS288 | | + | + | + | +++ | BA149 | ++ | ++ | ++ | ++ | + |
| KS284 | +++ | ++ | +++ | ++ | +++ | BA32 | ++ | ++ | +++ | | +++ |
| BA60 | ++ | ++ | +++ | ++ | +++ | BA55 | ++ | ++ | +++ | ++ | ++ |
| ZK318 | ++ | + | ++ | ++ | ++ | BA35 | ++ | | +++ | +++ | +++ |
| ZK320 | ++ | ++ | +++ | +++ | ++ | BA34 | ++ | ++ | +++ | ++ | +++ |
| ZK333 | +++ | ++ | +++ | +++ | +++ | BA38 | ++ | | +++ | +++ | +++ |
| ZK323 | +++ | ++ | +++ | +++ | +++ | BA40 | ++ | | ++ | ++ | +++ |
| ZK327 | +++ | ++ | +++ | +++ | +++ | BA41 | ++ | ++ | +++ | +++ | +++ |
| BA77d | ++ | ++ | +++ | ++ | ++ | BA14 | ++ | ++ | +++ | ++ | +++ |
| BA78d | ++ | ++ | +++ | ++ | +++ | BA12 | ++ | ++ | +++ | ++ | ++ |
| BA22 | +++ | +++ | +++ | +++ | +++ | BA30 | ++ | ++ | +++ | +++ | +++ |
| BA79d | +++ | +++ | +++ | +++ | +++ | ZK149 | + | ++ | ++ | ++ | + |
| BA85 | ++ | ++ | ++ | ++ | +++ | ZK126 | ++ | + | ++ | + | ++ |
| BA87 | +++ | +++ | +++ | +++ | +++ | ZK143 | + | + | + | + | + |
| ZK502 | ++ | ++ | +++ | ++ | +++ | ZK150 | ++ | + | + | + | ++ |
| ZK489 | +++ | +++ | +++ | +++ | +++ | ZK136 | ++ | + | + | ++ | +++ |
| ZK487 | +++ | +++ | +++ | +++ | +++ | ZK131 | + | + | + | + | + |
| Zk491 | +++ | +++ | +++ | +++ | ++ | ZK151 | + | + | + | + | +++ |
| Zk493 | +++ | +++ | +++ | +++ | +++ | ZK142 | + | + | + | + | + |
| BA62d | +++ | +++ | +++ | +++ | ++ | ZK139 | + | + | + | ++ | ++ |
| ZK450 | ++ | ++ | ++ | +++ | + | KS207 | ++ | + | +++ | +++ | +++ |
| ZK454 | ++ | ++ | ++ | +++ | +++ | KS208 | ++ | ++ | ++ | +++ | +++ |
| ZK469 | +++ | +++ | ++ | +++ | +++ | ZK102 | + | + | + | + | +++ |
| ZK471 | +++ | +++ | +++ | +++ | ++ | ZK157 | | | +++ | | +++ |
| ZK461 | +++ | ++ | +++ | ++ | ++ | ZK159 | ++ | ++ | ++ | ++ | +++ |
| ZK413 | ++ | +++ | +++ | +++ | +++ | ZK156 | ++ | ++ | ++ | ++ | +++ |
| ZK379 | +++ | ++ | +++ | +++ | +++ | KS63 | + | + | + | + | +++ |
| ZK421 | +++ | ++ | +++ | +++ | +++ | ZK158 | ++ | ++ | ++ | ++ | +++ |
| ZK403 | ++ | +++ | +++ | +++ | ++ | ZK147 | + | ++ | ++ | ++ | ++ |
| ZK405 | ++ | ++ | +++ | ++ | +++ | ZK155 | ++ | ++ | ++ | ++ | +++ |
| ZK432 | +++ | ++ | +++ | +++ | +++ | ZK162 | ++ | + | ++ | ++ | +++ |
| ZK434 | +++ | +++ | +++ | +++ | +++ | ZK165 | ++ | + | ++ | ++ | +++ |
| ZK465 | +++ | + | +++ | +++ | +++ | ZK161 | ++ | + | ++ | ++ | ++ |
| ZK377 | ++ | ++ | ++ | ++ | +++ | ZK167 | | | ++ | | ++ |
| ZK399 | ++ | ++ | +++ | ++ | +++ | ZK168 | | | ++ | | +++ |
| ZK401 | ++ | + | ++ | + | +++ | BA116 | + | ++ | ++ | ++ | +++ |
| BA62 | +++ | ++ | +++ | +++ | +++ | BA17 | +++ | ++ | +++ | ++ | +++ |
| ZK358 | +++ | ++ | +++ | +++ | +++ | BA134 | ++ | + | ++ | + | ++ |
| ZK452 | ++ | ++ | ++ | +++ | +++ | BA105 | ++ | ++ | +++ | ++ | +++ |
| ZK456 | +++ | ++ | ++ | +++ | +++ | BA122 | ++ | ++ | +++ | ++ | +++ |
| ZK463 | +++ | ++ | +++ | +++ | +++ | BA111 | +++ | ++ | +++ | +++ | +++ |
| ZK371 | +++ | +++ | +++ | +++ | +++ | BA102 | +++ | +++ | +++ | +++ | +++ |
| ZK409 | +++ | +++ | +++ | +++ | +++ | BA112 | +++ | ++ | +++ | +++ | +++ |
| ZK428 | +++ | +++ | +++ | +++ | +++ | BA118 | +++ | ++ | +++ | +++ | +++ |
| ZK430 | +++ | +++ | +++ | +++ | +++ | BA130 | +++ | ++ | +++ | +++ | +++ |
| ZK387 | ++ | ++ | ++ | ++ | +++ | BA132 | +++ | +++ | +++ | +++ | +++ |
| ZK389 | ++ | +++ | +++ | ++ | +++ | BA139 | ++ | ++ | +++ | + | ++ |
| ZK369 | ++ | + | ++ | ++ | ++ | BA158 | +++ | +++ | +++ | +++ | +++ |
| ZK385 | ++ | ++ | +++ | ++ | +++ | BA140 | + | ++ | + | + | ++ |
| ZK391 | ++ | ++ | +++ | ++ | +++ | BA141 | + | + | + | + | + |
| BA155_2 | +++ | +++ | +++ | +++ | +++ | BA146 | +++ | +++ | +++ | +++ | +++ |
| BA157_2 | +++ | +++ | +++ | +++ | +++ | BA142 | +++ | ++ | ++ | +++ | +++ |
| BA59 | +++ | ++ | +++ | +++ | +++ | BA145 | ++ | ++ | ++ | ++ | ++ |
| BA63 | | ++ | | +++ | +++ | BA147 | ++ | ++ | ++ | ++ | ++ |
| BA93 | +++ | ++ | +++ | +++ | +++ | BA148 | ++ | ++ | ++ | ++ | +++ |
| BA49 | ++ | + | ++ | ++ | ++ | BA143 | ++ | ++ | ++ | ++ | +++ |
| BA15 | +++ | ++ | +++ | +++ | +++ | BA144 | +++ | ++ | +++ | +++ | +++ |
| ZK321 | +++ | ++ | +++ | +++ | +++ | BA129 | ++ | + | ++ | + | ++ |
| ZK337 | +++ | +++ | +++ | +++ | +++ | BA131 | +++ | ++ | +++ | +++ | +++ |
| ZK347 | +++ | +++ | +++ | +++ | +++ | BA133 | +++ | +++ | +++ | +++ | +++ |
| ZK325 | +++ | +++ | +++ | +++ | +++ | BA120 | +++ | ++ | +++ | +++ | +++ |
| ZK349 | +++ | +++ | +++ | +++ | +++ | BA108 | +++ | ++ | +++ | ++ | +++ |
| ZK423 | +++ | +++ | +++ | +++ | +++ | BA121 | +++ | ++ | +++ | ++ | +++ |
| ZK411 | ++ | +++ | +++ | +++ | +++ | BA89 | +++ | ++ | +++ | +++ | +++ |
| ZK407 | ++ | +++ | +++ | ++ | ++ | BA94 | +++ | ++ | +++ | +++ | +++ |
| BA98 | +++ | +++ | +++ | +++ | +++ | BA135 | +++ | +++ | +++ | +++ | +++ |
| S1 | ++ | + | ++ | + | + | BA136 | +++ | ++ | +++ | ++ | +++ |
| BA23 | +++ | +++ | +++ | +++ | +++ | BA137 | +++ | +++ | +++ | +++ | +++ |
| ZK485 | +++ | +++ | +++ | ++ | ++ | BA138 | +++ | ++ | +++ | +++ | +++ |
| ZK495 | +++ | ++ | +++ | ++ | +++ | BA160 | ++ | ++ | +++ | +++ | +++ |
| ZK496 | ++ | ++ | +++ | ++ | +++ | BA157_3 | +++ | ++ | +++ | +++ | +++ |
| ZK494 | +++ | +++ | +++ | +++ | +++ | BA154 | +++ | +++ | +++ | +++ | +++ |
| BA90 | +++ | +++ | +++ | +++ | +++ | BA110 | +++ | ++ | +++ | +++ | +++ |
| ZK341 | ++ | ++ | +++ | +++ | ++ | BA115 | +++ | ++ | +++ | +++ | +++ |
| ZK343 | +++ | ++ | +++ | ++ | +++ | BA159 | +++ | +++ | +++ | +++ | ++ |
| ZK361 | ++ | ++ | +++ | +++ | +++ | BA119 | ++ | ++ | +++ | ++ | +++ |
| ZK359 | +++ | +++ | +++ | +++ | +++ | BA107 | ++ | ++ | +++ | ++ | +++ |
| ZK362 | +++ | +++ | +++ | +++ | ++ | BA124 | ++ | ++ | +++ | ++ | +++ |
| BA64 | + | + | ++ | + | ++ | BA161 | + | + | + | + | +++ |
| BA65 | + | + | ++ | + | ++ | BA162 | ++ | ++ | ++ | ++ | +++ |
| ZK305 | +++ | +++ | +++ | +++ | +++ | BA24dd | ++ | ++ | +++ | ++ | +++ |
| ZK306 | ++ | + | ++ | ++ | ++ | BA43 | +++ | | +++ | +++ | +++ |
| BA66 | ++ | ++ | +++ | ++ | +++ | BA91 | ++ | ++ | ++ | ++ | +++ |
| BA48 | ++ | ++ | +++ | ++ | +++ | BA92 | ++ | ++ | +++ | ++ | +++ |
| ZK133 | + | + | + | ++ | ++ | BA86 | ++ | ++ | ++ | +++ | +++ |
| BA20dd | ++ | ++ | ++ | ++ | +++ | BA88 | + | + | ++ | +++ | +++ |
| BA20d | ++ | ++ | +++ | +++ | +++ | BA96 | ++ | + | +++ | +++ | +++ |
| BA20 | ++ | | ++ | ++ | ++ | BA97 | ++ | ++ | +++ | +++ | +++ |
| BA99 | ++ | + | ++ | + | ++ | BA44 | ++ | +++ | ++ | ++ | ++ |
| BA81dd | +++ | ++ | +++ | +++ | + | BA156 | ++ | ++ | ++ | ++ | +++ |
| BA81d | +++ | ++ | +++ | +++ | +++ | BA95 | ++ | ++ | +++ | +++ | +++ |
| ZK137 | ++ | + | ++ | ++ | +++ | ZK129 | ++ | ++ | ++ | ++ | +++ |
| ZK135 | ++ | + | + | ++ | ++ | BA54 | ++ | ++ | ++ | ++ | +++ |
| ZK130 | + | + | + | + | + | ZK152 | ++ | ++ | ++ | ++ | +++ |
| ZK128 | ++ | + | ++ | ++ | ++ | BA42 | ++ | ++ | ++ | ++ | +++ |
| BA26 | ++ | ++ | +++ | +++ | ++ | PIK294 | ++ | +++ | +++ | +++ | ++ |
| SU11248 | ++ | + | ++ | ++ | + | Iressa | + | + | + | + | + |
| BAY43-9006 | ++ | + | + | + | + | PIK103 | +++ | +++ | +++ | +++ | +++ |
| Dasatinib | ++ | + | ++ | + | ++ | PIK90 | +++ | +++ | +++ | +++ | +++ |

TABLE 3
IC50 values for the Compounds of Table 1 in-vitro assays against
Abl, Hck, Src, Src (T/1), VEGFR, EGFR, and EphB4..

| | | | | Src | | | |
|---|---|---|---|---|---|---|---|
| Cpd | Abl | Hck | Src | (T/I) | VEGFR | EGFR | EphB4 |
| KS84 | +++ | +++ | +++ | ++ | +++ | +++ | +++ |
| BA56 | ++ | +++ | +++ | ++ | ++ | +++ | ++ |
| KS284 | +++ | +++ | +++ | ++ | +++ | +++ | +++ |
| BA60 | ++ | +++ | +++ | +++ | ++ | + | ++ |
| ZK318 | ++ | ++ | +++ | ++ | + | | ++ |
| ZK320 | +++ | +++ | +++ | ++ | +++ | ++ | ++ |
| ZK333 | +++ | +++ | +++ | ++ | +++ | ++ | ++ |
| ZK323 | +++ | +++ | +++ | +++ | +++ | +++ | +++ |
| ZK327 | +++ | +++ | +++ | ++ | +++ | ++ | ++ |
| BA77d | +++ | +++ | +++ | ++ | ++ | ++ | +++ |
| BA78d | +++ | +++ | +++ | ++ | +++ | ++ | ++ |
| BA22 | +++ | +++ | +++ | ++ | +++ | +++ | +++ |
| BA79d | +++ | +++ | +++ | ++ | +++ | +++ | +++ |
| BA85 | ++ | +++ | +++ | ++ | ++ | ++ | ++ |
| BA87 | +++ | +++ | +++ | +++ | +++ | +++ | +++ |
| ZK502 | +++ | +++ | +++ | ++ | ++ | ++ | + |
| ZK489 | +++ | +++ | +++ | + | ++ | + | ++ |
| ZK487 | +++ | +++ | +++ | ++ | +++ | +++ | +++ |
| Zk491 | +++ | +++ | +++ | ++ | +++ | +++ | +++ |
| Zk493 | +++ | +++ | +++ | ++ | +++ | +++ | +++ |
| BA62d | +++ | +++ | +++ | +++ | +++ | ++ | +++ |
| ZK450 | +++ | +++ | +++ | + | ++ | + | ++ |
| ZK454 | +++ | ++ | +++ | + | ++ | ++ | + |
| ZK469 | +++ | +++ | +++ | + | ++ | ++ | ++ |
| ZK471 | +++ | +++ | +++ | + | ++ | ++ | + |
| ZK461 | +++ | +++ | +++ | + | +++ | ++ | ++ |
| ZK413 | +++ | +++ | +++ | + | ++ | ++ | |
| ZK379 | +++ | +++ | +++ | ++ | +++ | +++ | +++ |
| ZK421 | +++ | +++ | +++ | +++ | +++ | +++ | +++ |
| ZK403 | +++ | +++ | +++ | ++ | +++ | ++ | +++ |
| ZK405 | +++ | +++ | +++ | ++ | ++ | ++ | + |
| ZK432 | +++ | +++ | +++ | ++ | +++ | ++ | +++ |
| ZK434 | +++ | +++ | +++ | ++ | +++ | ++ | +++ |
| ZK465 | +++ | +++ | +++ | + | +++ | +++ | +++ |
| ZK377 | ++ | ++ | +++ | + | ++ | + | + |
| ZK399 | +++ | +++ | +++ | + | + | + | ++ |
| ZK401 | +++ | +++ | +++ | + | + | + | + |
| BA62 | +++ | +++ | +++ | + | + | ++ | +++ |
| ZK358 | +++ | +++ | +++ | ++ | +++ | +++ | +++ |
| ZK452 | +++ | +++ | +++ | ++ | +++ | ++ | ++ |
| ZK456 | +++ | +++ | +++ | + | ++ | ++ | ++ |
| ZK463 | +++ | +++ | +++ | ++ | +++ | ++ | ++ |
| ZK371 | +++ | +++ | +++ | ++ | +++ | +++ | +++ |
| ZK409 | +++ | +++ | +++ | ++ | +++ | ++ | +++ |
| ZK428 | +++ | +++ | +++ | ++ | +++ | ++ | +++ |
| ZK430 | +++ | +++ | +++ | ++ | +++ | ++ | +++ |
| ZK387 | +++ | +++ | +++ | + | ++ | ++ | ++ |
| ZK389 | +++ | +++ | +++ | ++ | +++ | ++ | +++ |
| ZK369 | ++ | ++ | +++ | + | ++ | ++ | + |
| ZK385 | ++ | +++ | +++ | + | ++ | + | ++ |
| ZK391 | ++ | +++ | +++ | + | ++ | ++ | ++ |
| BA155_2 | ++ | +++ | +++ | ++ | ++ | ++ | ++ |
| BA157_2 | ++ | ++ | ++ | ++ | ++ | ++ | ++ |
| BA59 | +++ | +++ | +++ | +++ | ++ | ++ | +++ |
| BA63 | ++ | +++ | +++ | ++ | ++ | ++ | ++ |
| BA93 | ++ | +++ | +++ | ++ | ++ | ++ | ++ |
| BA49 | ++ | +++ | +++ | ++ | + | ++ | +++ |
| BA15 | ++ | +++ | +++ | ++ | + | +++ | ++ |
| ZK321 | +++ | +++ | +++ | ++ | ++ | + | ++ |
| ZK337 | +++ | +++ | +++ | | | + | ++ |
| ZK347 | +++ | +++ | +++ | ++ | +++ | ++ | ++ |
| ZK325 | +++ | +++ | +++ | ++ | +++ | ++ | +++ |
| ZK349 | +++ | +++ | +++ | +++ | +++ | +++ | +++ |
| ZK423 | +++ | +++ | +++ | ++ | +++ | +++ | +++ |
| ZK411 | ++ | ++ | +++ | + | ++ | ++ | ++ |
| ZK407 | ++ | +++ | +++ | + | +++ | ++ | + |
| BA98 | +++ | +++ | +++ | ++ | +++ | +++ | ++ |
| S1 | +++ | + | ++ | + | + | + | + |
| BA23 | ++ | ++ | +++ | ++ | + | ++ | ++ |
| ZK485 | ++ | +++ | +++ | + | + | ++ | ++ |
| ZK495 | ++ | +++ | ++ | ++ | + | ++ | ++ |
| ZK496 | ++ | ++ | ++ | ++ | ++ | ++ | ++ |
| ZK494 | +++ | +++ | +++ | ++ | ++ | ++ | ++ |
| BA90 | ++ | +++ | +++ | ++ | ++ | ++ | ++ |
| ZK341 | ++ | ++ | + | + | + | + | + |
| ZK343 | ++ | +++ | +++ | + | ++ | ++ | ++ |
| ZK361 | ++ | ++ | ++ | + | + | ++ | + |
| ZK359 | +++ | +++ | +++ | ++ | ++ | ++ | +++ |
| ZK362 | ++ | +++ | +++ | + | + | ++ | + |
| BA64 | +++ | +++ | +++ | ++ | + | ++ | +++ |
| BA65 | ++ | +++ | +++ | ++ | ++ | ++ | ++ |
| ZK305 | ++ | ++ | ++ | ++ | + | + | ++ |
| ZK306 | + | ++ | ++ | ++ | ++ | ++ | + |
| BA66 | ++ | ++ | ++ | + | | + | ++ |
| BA48 | ++ | +++ | +++ | ++ | ++ | ++ | ++ |
| BA20dd | +++ | +++ | +++ | ++ | ++ | ++ | ++ |
| BA20d | +++ | +++ | +++ | ++ | ++ | ++ | ++ |
| BA20 | +++ | +++ | +++ | +++ | ++ | +++ | + |
| BA99 | ++ | +++ | +++ | ++ | +++ | ++ | ++ |
| BA81dd | +++ | +++ | +++ | ++ | +++ | +++ | +++ |
| BA81d | +++ | +++ | +++ | ++ | +++ | +++ | +++ |
| BA26 | ++ | ++ | +++ | ++ | + | + | + |
| BA46 | ++ | ++ | ++ | + | ++ | ++ | ++ |
| BA45 | ++ | +++ | +++ | ++ | ++ | ++ | + |
| BA39 | ++ | ++ | ++ | ++ | ++ | + | ++ |
| BA150 | ++ | ++ | ++ | + | ++ | + | ++ |
| BA151 | ++ | ++ | ++ | + | + | ++ | ++ |
| BA21 | ++ | ++ | ++ | + | + | + | + |
| BA52 | ++ | ++ | ++ | + | + | + | + |
| BA53 | ++ | ++ | ++ | + | + | + | + |
| BA31 | ++ | ++ | ++ | + | + | ++ | + |
| BA152 | +++ | +++ | +++ | + | ++ | ++ | ++ |
| BA149 | ++ | +++ | +++ | ++ | ++ | ++ | ++ |
| BA32 | ++ | ++ | ++ | + | + | + | ++ |
| BA55 | ++ | ++ | ++ | + | + | ++ | ++ |
| BA35 | ++ | ++ | ++ | ++ | ++ | ++ | ++ |
| BA34 | ++ | ++ | ++ | ++ | ++ | ++ | ++ |
| BA38 | ++ | ++ | ++ | ++ | + | + | + |
| BA40 | ++ | ++ | ++ | ++ | ++ | ++ | + |
| BA41 | ++ | ++ | ++ | ++ | ++ | + | ++ |
| BA14 | ++ | ++ | ++ | ++ | + | + | ++ |
| BA12 | ++ | ++ | ++ | ++ | ++ | ++ | ++ |
| BA30 | ++ | ++ | ++ | ++ | ++ | +++ | ++ |
| KS208 | ++ | +++ | ++ | ++ | ++ | +++ | ++ |
| BA116 | ++ | +++ | ++ | | +++ | ++ | ++ |
| BA17 | +++ | +++ | +++ | +++ | +++ | +++ | +++ |
| BA134 | ++ | ++ | ++ | ++ | + | + | ++ |
| BA105 | ++ | +++ | ++ | ++ | ++ | ++ | ++ |
| BA122 | +++ | +++ | +++ | ++ | ++ | +++ | +++ |
| BA111 | + | + | ++ | ++ | ++ | ++ | ++ |
| BA102 | +++ | +++ | +++ | ++ | +++ | +++ | +++ |
| BA112 | ++ | +++ | +++ | ++ | ++ | ++ | ++ |
| BA118 | ++ | ++ | + | ++ | ++ | + | ++ |
| BA130 | ++ | +++ | ++ | ++ | +++ | + | ++ |
| BA132 | ++ | +++ | ++ | + | ++ | ++ | ++ |
| BA139 | ++ | ++ | ++ | + | ++ | ++ | + |
| BA158 | +++ | +++ | +++ | ++ | ++ | ++ | ++ |
| BA140 | ++ | ++ | ++ | ++ | + | ++ | ++ |
| BA141 | + | ++ | + | + | + | ++ | + |
| BA146 | ++ | ++ | ++ | + | ++ | ++ | ++ |
| BA142 | ++ | ++ | +++ | + | ++ | ++ | ++ |
| BA145 | + | ++ | ++ | + | + | + | ++ |
| BA147 | ++ | ++ | ++ | + | ++ | + | ++ |
| BA148 | + | ++ | ++ | + | + | ++ | + |
| BA143 | ++ | ++ | ++ | ++ | + | ++ | ++ |
| BA144 | ++ | ++ | +++ | + | ++ | + | ++ |
| BA129 | ++ | + | +++ | + | ++ | ++ | ++ |
| BA131 | ++ | ++ | 11 | + | ++ | + | ++ |
| BA133 | ++ | ++ | +++ | + | + | ++ | ++ |
| BA120 | +++ | +++ | +++ | ++ | +++ | ++ | ++ |
| BA108 | +++ | +++ | +++ | +++ | +++ | +++ | +++ |
| BA121 | +++ | +++ | +++ | +++ | +++ | +++ | +++ |
| BA89 | +++ | +++ | +++ | +++ | +++ | +++ | +++ |
| BA94 | +++ | +++ | +++ | +++ | +++ | +++ | +++ |
| BA135 | +++ | +++ | +++ | ++ | +++ | +++ | + |
| BA136 | +++ | +++ | +++ | ++ | +++ | | ++ |
| BA137 | +++ | +++ | +++ | +++ | +++ | ++ | ++ |
| BA138 | +++ | +++ | +++ | +++ | +++ | +++ | ++ |
| BA160 | +++ | +++ | +++ | ++ | ++ | +++ | ++ |
| BA157_3 | ++ | +++ | +++ | ++ | + | ++ | ++ |
| BA154 | +++ | +++ | +++ | ++ | ++ | ++ | ++ |
| BA110 | +++ | +++ | +++ | ++ | +++ | ++ | ++ |
| BA115 | +++ | +++ | +++ | +++ | +++ | +++ | |
| BA159 | +++ | +++ | +++ | ++ | +++ | ++ | ++ |
| BA119 | ++ | +++ | +++ | ++ | ++ | ++ | ++ |
| BA107 | +++ | +++ | +++ | ++ | +++ | +++ | +++ |
| BA124 | +++ | +++ | +++ | +++ | +++ | +++ | +++ |
| BA161 | ++ | + | ++ | + | + | ++ | + |
| BA162 | ++ | ++ | ++ | + | + | ++ | ++ |
| BA24dd | ++ | ++ | ++ | ++ | ++ | ++ | ++ |
| BA43 | ++ | +++ | ++ | ++ | ++ | ++ | ++ |
| BA91 | ++ | ++ | +++ | ++ | ++ | ++ | ++ |
| BA92 | ++ | +++ | +++ | ++ | ++ | ++ | ++ |
| BA86 | +++ | +++ | +++ | +++ | +++ | +++ | +++ |
| BA88 | +++ | +++ | +++ | +++ | +++ | +++ | +++ |
| BA96 | +++ | +++ | +++ | ++ | ++ | +++ | +++ |
| BA97 | +++ | +++ | +++ | ++ | ++ | +++ | +++ |
| BA44 | ++ | ++ | ++ | ++ | + | ++ | + |
| BA156 | ++ | ++ | ++ | ++ | ++ | +++ | ++ |
| BA95 | ++ | +++ | ++ | + | + | ++ | ++ |
| BA54 | ++ | ++ | +++ | ++ | ++ | ++ | ++ |
| BA42 | + | ++ | ++ | ++ | ++ | + | ++ |
| SU11248 | +++ | +++ | +++ | +++ | +++ | ++ | + |
| BAY43-9006 | +++ | +++ | +++ | +++ | +++ | + | ++ |
| Dasatinib | | | | ++ | + | +++ | |
| Iressa | ++ | +++ | +++ | ++ | ++ | | ++ |
| PIK103 | + | + | + | + | + | + | + |
| PIK90 | + | + | + | + | + | + | + |
| PIK294 | ++ | + | ++ | + | + | + | + |

TABLE 4
IC50 Values for the Compounds of Table 1 in-vitro assays
against cKIT, Tie2, FLT3, PDGFR, RET, IT, and mTOR

| Cpd | cKIT | Tie2 | FLT3 | PDGFR | RET | IR | mTOR |
|---|---|---|---|---|---|---|---|
| ZK358 | +++ | ++ | +++ | +++ | +++ | ++ | +++ |
| ZK487 | +++ | +++ | +++ | +++ | +++ | + | +++ |
| ZK349 | +++ | +++ | +++ | +++ | +++ | ++ | +++ |
| ZK494 | +++ | ++ | ++ | +++ | +++ | + | +++ |
| BA102 | +++ | +++ | +++ | +++ | +++ | + | +++ |
| BA121 | +++ | +++ | +++ | +++ | | ++ | +++ |
| KS84 | +++ | + | +++ | +++ | +++ | ++ | ++ |
| SU11248 | +++ | ++ | +++ | +++ | +++ | ++ | + |
| BAY43-9006 | +++ | +++ | + | +++ | +++ | ++ | + |
| Dasatinib | +++ | ++ | +++ | +++ | ++ | + | + |
| Iressa | + | ++ | +++ | +++ | ++ | + | + |

In Tables 2-4 above, a +++ indicates an IC50 of less than 1 μM; a ++ indicates an IC50 of from 1 μM to 50 μM; and a + indicates and IC50 of more than 50 μM.

TABLE 5
Exemplary compounds of Formula XV and their IC50 values for in-vitro assays against the
p110α, p110β, p110χ, p110δ isoforms of PI3 kinase.

| Compound | | IC50 | | | |
|---|---|---|---|---|---|
| MW | Structure | p110α | p110β | p110γ | p110δ |
| S1 (509.9464) | 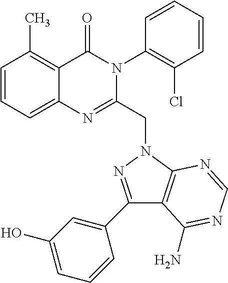 | + | ++ | +++ | +++ |
| S2 (523.33) | 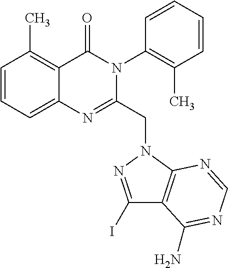 | ++ | ++ | +++ | +++ |
| S3 (489.53) | 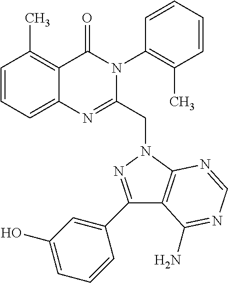 | + | +++ | +++ | +++ |
| S4 (557.63) | 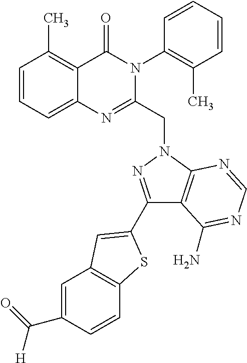 | + | + | ++ | +++ |
| S5 (521.59) | 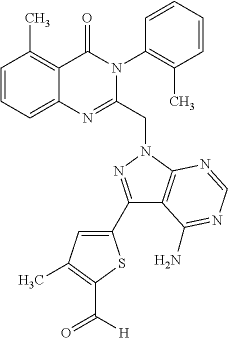 | + | ++ | +++ | +++ |
| S6 (533.58) | 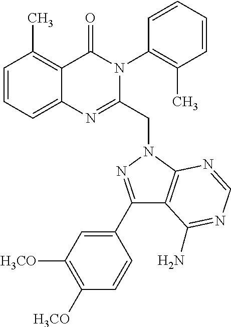 | + | ++ | +++ | +++ |
| S7 (565.62) | 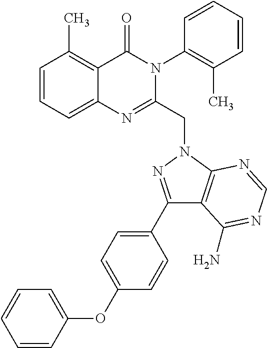 | + | ++ | ++ | +++ |
| S8 (579.65) | 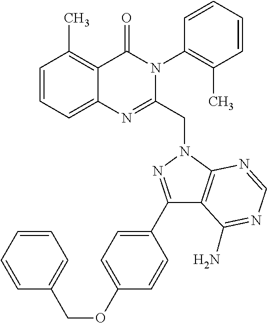 | ++ | ++ | ++ | +++ |
| S9 (507.52) | 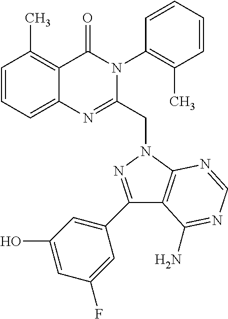 | ++ | +++ | +++ | +++ |
| S10 (507.52) | 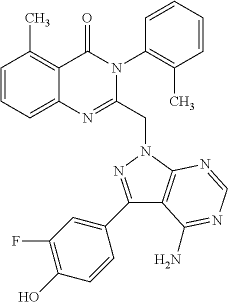 | ++ | +++ | +++ | +++ |
| S11 (503.55) | 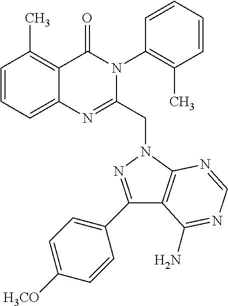 | ++ | ++ | +++ | +++ |
| S12 (523.97) | 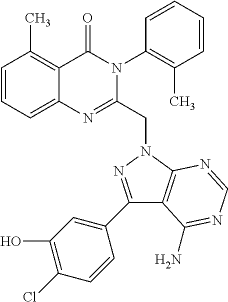 | ++ | ++ | +++ | +++ |
| S13 (507.52) | 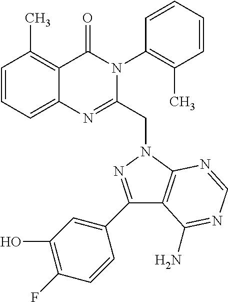 | ++ | ++ | +++ | +++ |
| S14 (512.56) | 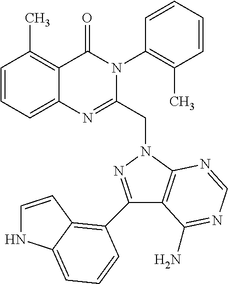 | + | ++ | +++ | +++ |
| S15 (512.56) | 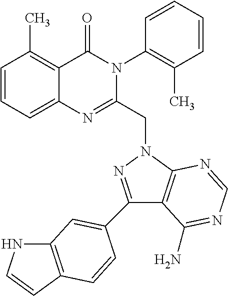 | + | ++ | ++ | +++ |
| S16 (521.55) | 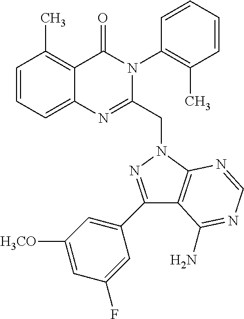 | + | ++ | ++ | +++ |
| S17 (521.55) | 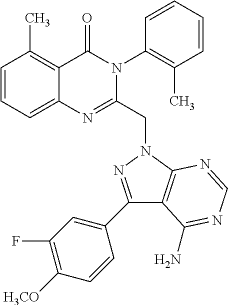 | ++ | ++ | +++ | +++ |
| S18 (608.52) | 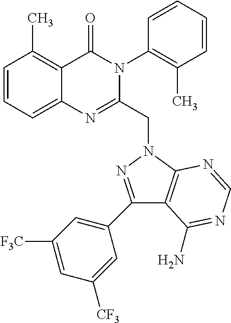 | + | + | + | ++ |
| S19 (461.52) | 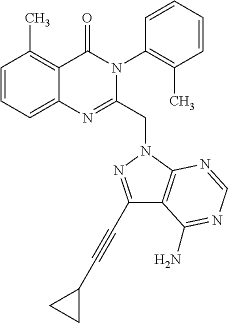 | + | ++ | ++ | +++ |
| S20 (513.55) | 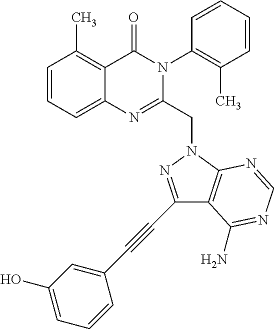 | ++ | ++ | +++ | +++ |
| S21 (512.56) | 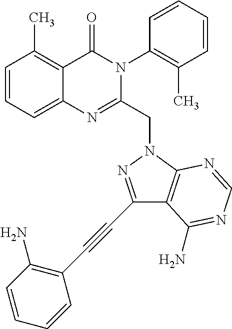 | + | ++ | ++ | +++ |
| S22 (498.54) | 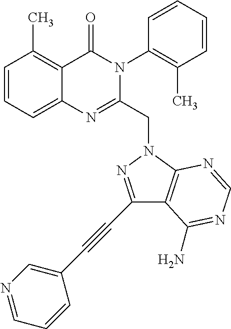 | + | ++ | ++ | +++ |
| S23 (498.54) | 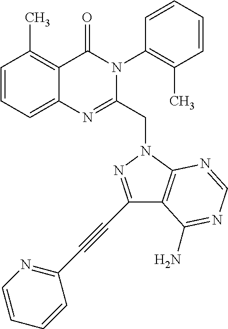 | + | ++ | +++ | +++ |
| S24 (524.58) | 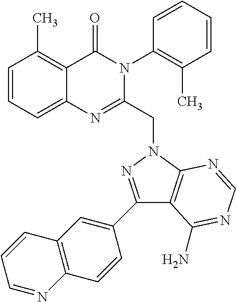 | ++ | ++ | +++ | +++ |
| S25 (451.48) | 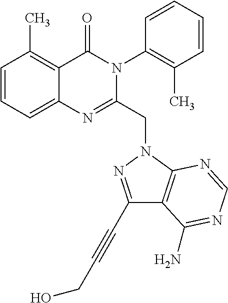 | ++ | +++ | ++ | +++ |
| S26 (464.52) | 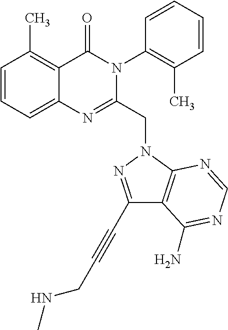 | | | | +++ |
| S27 (478.55) | 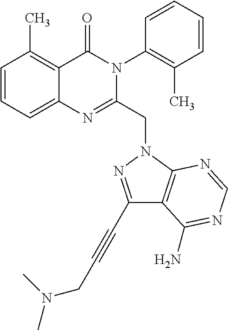 | | | | ++ |
| S28 (465.51) | 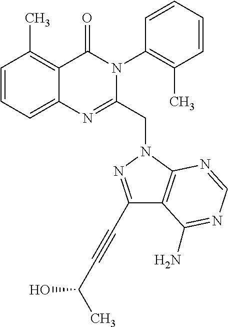 | + | ++ | ++ | +++ |
| S29 (465.51) | 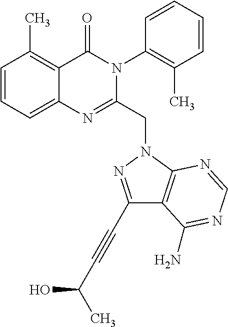 | ++ | +++ | +++ | +++ |
| S30 (493.52) | 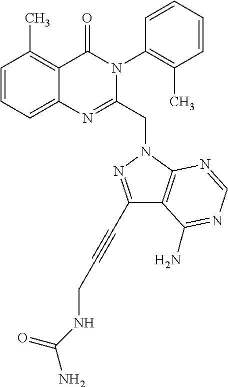 | ++ | + | ++ | +++ |
| S31 (488.54) | 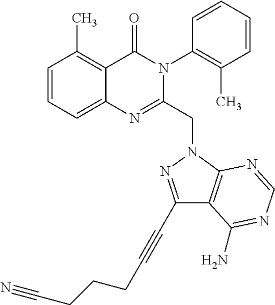 | | | | +++ |
| S32 | 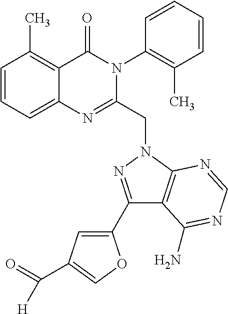 | | | | |
| S33 | 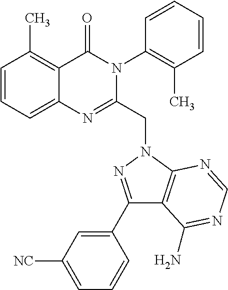 | | | | |
| S34 | 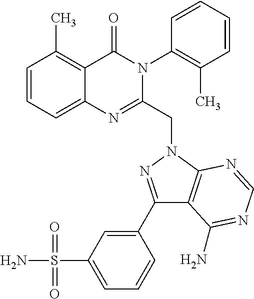 | | | | |

The symbol +++ represents an IC50 of less than 1 μM; the symbol ++ represents an IC 50 value from 1 μM to 100 μM; and + represents an IC50 value of more than 100 μM.

### SYNTHETIC PREPARATION AND EXAMPLES

### Pyrazolopyrimidine Inhibitors

The synthesis of one class of compounds disclosed herein as useful in the methods of the invention, are synthesized as illustrated in the following schemes. The examples are described for specific embodiments but the synthetic methods can be used for the other compounds of Formula I.

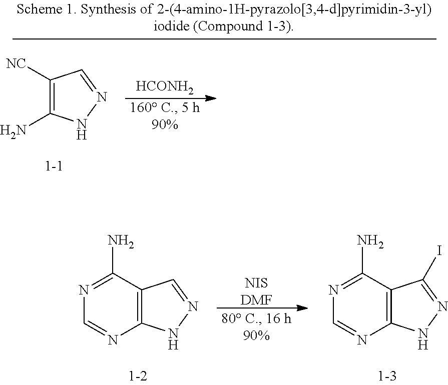

Scheme 1 depicts the synthesis of 2-(4-amino-1H-pyrazolo[3,4-d]pyrimidin-3-yl)iodide. Cyano substituted aminopyrazole 1-1 is heated with formamide at 160° C. for 5 hours to yield 2-(4-amino-1H-pyrazolo[3,4-d]pyrimidine (compound 1-2) in 90% yield. This intermediate is reacted with N-iodosuccinimide in dimethylformamide at 80° C. for 16 hours, to produce 2-(4-amino-1H-pyrazolo[3,4-d]pyrimidin-3-yl)iodide (Cpd. 1-3) in 90% yield.

### Synthesis of 2-(4-amino-1-isopropyl-1H-pyrazolo[3,4-d]pyrimidin-3-yl)-1H-indol-5-ol (Compound 2-4)

Compound 2-4 is synthesized as shown in Scheme 2. Compound 1-3 is reacted with isopropyl bromide in dimethylformamide with potassium carbonate at 80° C., to provide the 1-isopropyl pyrazolopyrimidine intermediate, compound 2-1. This intermediate is reacted with the protected indolyl boronic acid species 2-2, using tetrakistriphenylphosphine palladium catalysis in DME-water solvent at 80° C. for 4-5 hours, to produce the Suzuki coupling product, compound 2-3. Removal of the protecting groups with acid in dioxane yields the product (Cpd. 2-4).

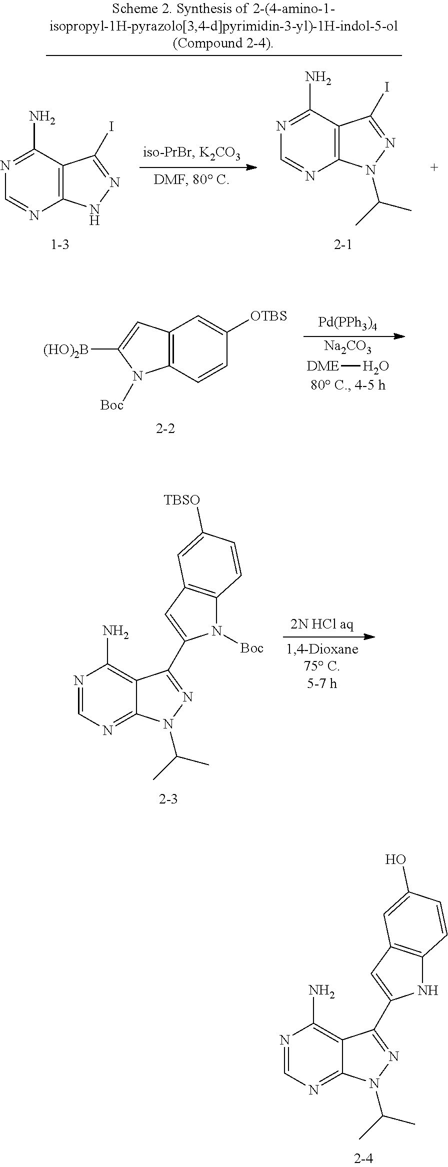

### 2-(4-amino-1-isopropyl-1H-pyrazolo[3,4-d]pyrimidin-3-yl)-1H-indole (Compound 3-2)

Synthesis of 2-(4-amino-1-isopropyl-1H-pyrazolo[3,4-d]pyrimidin-3-yl)-1H-indole (Compound 3-3) is accomplished via the same reactions except that boronic acid 3-1 is used, as shown in Scheme 3.

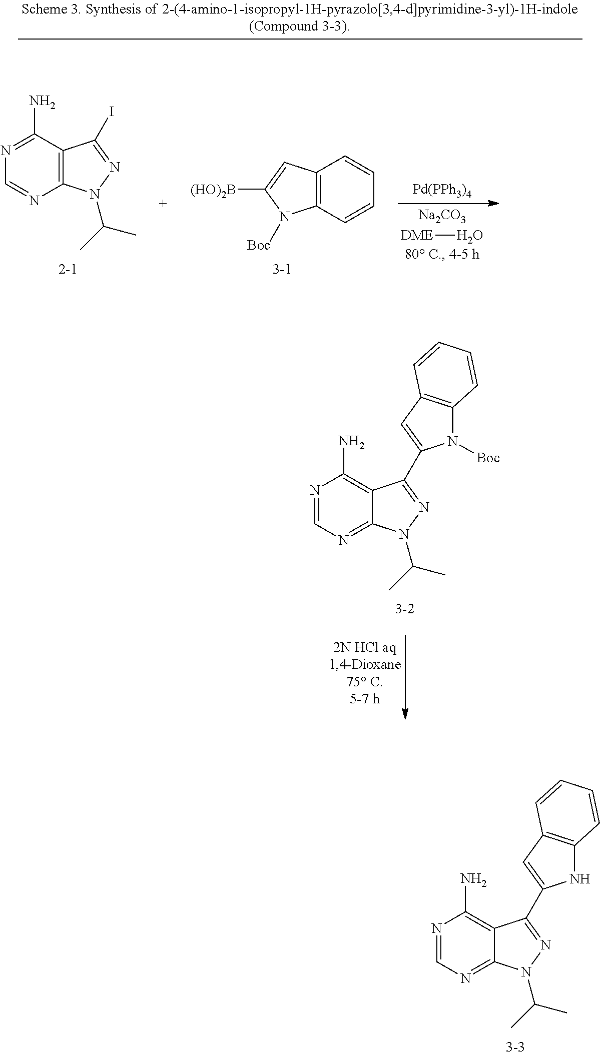

The synthesis of 2-(4-amino-1-isopropyl-1H-pyrazolo[3,4-d]pyrimidin-3-yl)-1H-indol-7-ol is accomplished via the same reactions as in Schemes 1 and 2, using a 7-tert-butyldimethylsilyloxy (TBS) indolyl boronic acid instead of the 5-TBSO indolyl species illustrated.

Alternatively, 2-(4-amino-1-isopropyl-1H-pyrazolo[3,4-d]pyrimidin-3-yl)-1H-indol-5-ol (Compound 2-4 and 3-4) is synthesized via methoxy protected intermediates as shown in Scheme 3-B. 5-Methoxy indolyl boronic acid, compound 3-1 is coupled to pyrazolopyrimidine iodide (compound 2-1) using palladium acetate and triphenylphosphine in the presence of sodium carbonate base to provide intermediate 3-6. Along with the desired product, some partially deprotected product is also formed. The crude mixture is taken into the next step for complete Boc deprotection. Deprotection is accomplished with aqueous HCl in ethanol solution and compound 3-7 is isolated as the HCl salt. In the last step, the salt is brought to pH 8 in aqueous potassium carbonate to obtain the free base. This material is treated with boron tribromide to remove the methyl ether protection and yield the final product, Compound 3-4.

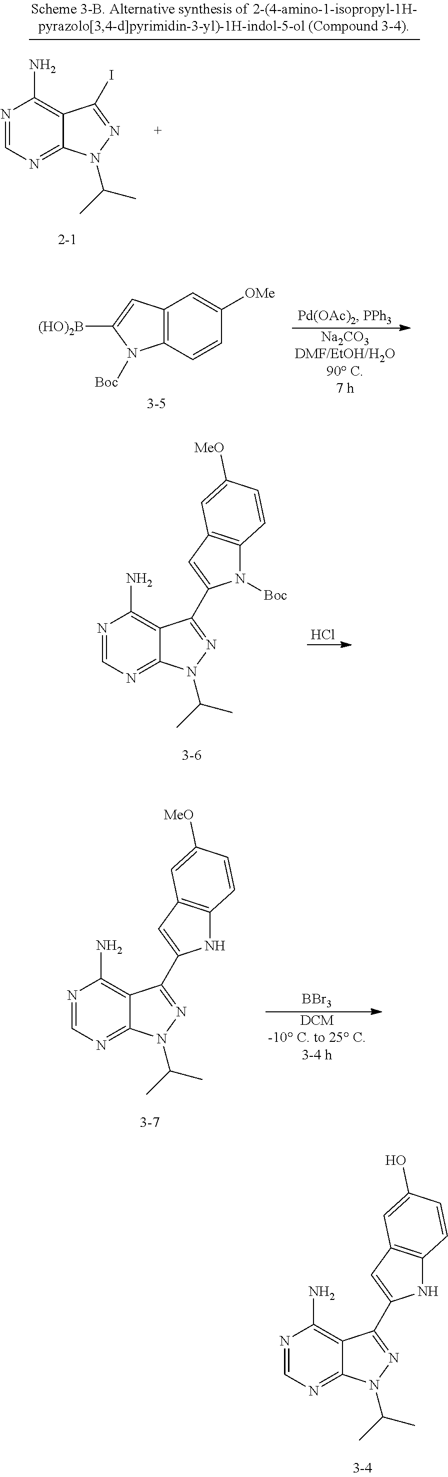

The synthesis of 2-(4-amino-1-isopropyl-1H-pyrazolo[3,4-d]pyrimidin-3-yl)-1H-indol-6-ol is accomplished via the same reactions as in Schemes 1 and 2, using a 6-tert-butyldimethylsilyloxy (TBS) indolyl boronic acid instead of the 5-TBSO indolyl species illustrated or it is synthesized via reactions as shown in Scheme 3-B, using a 6-methoxy indolyl boronic acid instead of the 5-methoxy indolyl boronic acid illustrated.

### Quinazolinone Inhibitors.

The following examples are meant to illustrate certain embodiments of the compounds of Formula XV, and not to limit the scope of the invention.

### A. Exemplary Synthesis Scheme

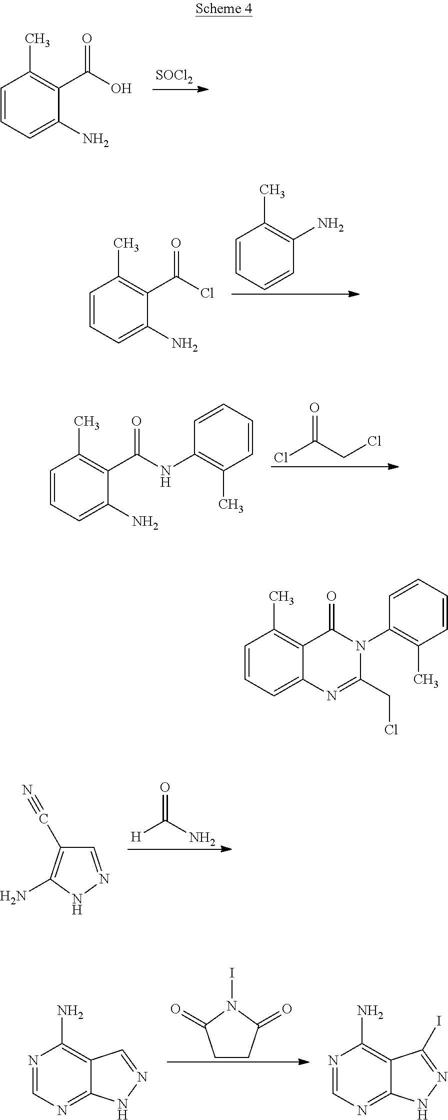

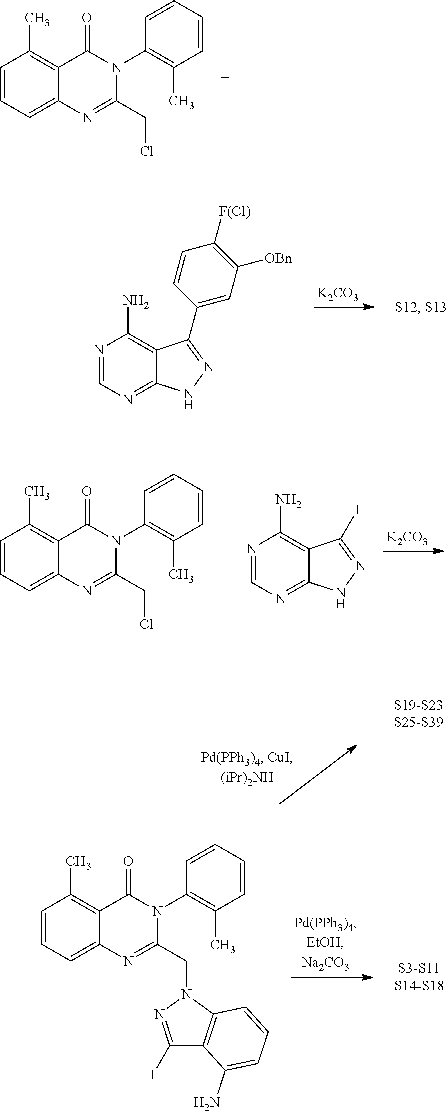

Scheme 4 illustrates synthetic routes to certain compounds listed in Table 5. Using the information provided in Scheme 4, and the detailed synthesis information of certain compounds provided below, one skilled in the art would immediately recognize the synthetic routes to the compounds of the present invention.

### B. Detailed Synthesis of Certain Compounds

### Synthesis of 2-amino-6-methyl-N-o-tolylbenzamide

2-amino-6-methylbenzoic acid (25 g, 165 mmol) was dissolved in benzene (250 mL). Thionyl chloride (37.5 mL, 500 mmol) was added and the reaction heated to reflux overnight. The following day the reaction was concentrated in vacuo, and then taken up twice in benzene (200 mL) and solvent removed in vacuo again to give a black oil. The oil was dissolved in CHCl₃ (400 mL), o-toluidine (44 mL, 412 mmol) was added and the reaction heated to reflux. Reaction was complete after two hours, and the product was purified by three silica gel chromatographies (15% EtOAc/Hexanes) to yield a tan solid (29 g, 73.4% yield). LR-ESI MS (M+H)⁺ m/z calcd 241.1, found 240.9.

### Synthesis of 2-(chloromethyl)-5-methyl-3-o-tolylquinazolin-4(3H)-one

Chloroacetylchloride (29 mL, 363 mmol) was added to a solution of 2-amino-6-methyl-N-o-tolylbenzamide (29 g, 121 mmol) in acetic acid (600 mL) and the reaction heated to reflux. After two hours the reaction was cooled to RT, and concentrated in vacuo. The product was purified by three silica gel chromatographies (twice in 15% EtOAc/Hexanes followed by 10% diethylether/hexanes) to yield a white solid (8.3 g, 23% yield). LR-ESI MS (M+H)⁺ m/z calcd 299.1, found 298.8.

### Synthesis of 2-((4-amino-1H-pyrazolo[3,4-d]pyrimidin-1-yl)methyl)-5-methyl-3-o-tolylquinazolin-4(3H)-one

2-(chloromethyl)-5-methyl-3-o-tolylquinazolin-4(3H)-one (0.15 g, 0.5 mmol) and 1H-pyrazolo[3,4-d]pyrimidin-4-amine (0.101 g, 0.05 mmol) were added to DMF (10 mL) and K₂CO₃ (0.138 g, 1 mmol) and allowed to stir at RT in the dark for 24 hours. The product was precipitated by addition of water (800 mL) and collected by filtration. The precipitate was further purified by RP-HPLC (MeCN:H₂O:0.1% TFA). LR-ESI MS (M+H)⁺ m/z calcd 398.2, found 398.1.

### Synthesis of Compound S2

2-(chloromethyl)-5-methyl-3-o-tolylquinazolin-4(3H)-one (3 g, 10.0 mmol) and 3-iodo-1H-pyrazolo[3,4-d]pyrimidin-4-amine (3.91 g, 15.05 mmol) were added to DMF (50 mL) and K₂CO₃ (2.77 g, 20 mmol) and allowed to stir at RT in the dark for 24 hours. The product was precipitated by addition of water (900 mL) and collected by filtration. The precipitate was further purified by silica gel chromatography (2% MeOH/CH₂Cl₂). LR-ESI MS (M+H)⁺ m/z calcd 524.1, found 523.9.

### Synthesis of Compound S3

2-((4-amino-3-iodo-1H-pyrazolo[3,4-d]pyrimidin-1-yl)methyl)-5-methyl-3-o-tolylquinazolin-4(3H)-one (50 mg, 0.096 mmol), m-phenol boronic acid (14.5 mg, 0.105 mmol) and tetrakis(triphenylphosphine)palladium (22 mg, 0.019 mmol) were dissolved in a solution of DME (10 mL), EtOH (1.6 mL) and saturated aqueous Na₂CO₃ (2.75 mL). The reaction was heated to reflux overnight under an argon atmosphere. The following day the reaction was poured into water, and the aqueous phase extracted three times with CH₂Cl₂. The organic extract was concentrated in vacuo and purified by RP-HPLC (MeCN:H₂O:0.1% TFA). LR-ESI MS (M+H)⁺ m/z calcd 490.2, found 490.1.

### Synthesis of Compound S4

2-((4-amino-3-iodo-1H-pyrazolo[3,4-d]pyrimidin-1-yl)methyl)-5-methyl-3-o-tolylquinazolin-4(3H)-one (50 mg, 0.096 mmol), 5-formylbenzo[b]thiophene-2-boronic ester (30.3 mg, 0.105 mmol) and tetrakis(triphenylphosphine)palladium (22 mg, 0.019 mmol) were dissolved in a solution of DME (10 mL), EtOH (1.6 mL) and saturated aqueous Na₂CO₃ (2.75 mL). The reaction was heated to reflux overnight under an argon atmosphere. The following day the reaction was poured into water, and the aqueous phase extracted three times with CH₂Cl₂. The organic extract was concentrated in vacuo and purified by RP-HPLC (MeCN:H₂O:0.1% TFA). LR-ESI MS (M+H)⁺ m/z calcd 558.2, found 558.0.

### Synthesis of Compound S5

2-((4-amino-3-iodo-1H-pyrazolo[3,4-d]pyrimidin-1-yl)methyl)-5-methyl-3-o-tolylquinazolin-4(3H)-one (50 mg, 0.096 mmol), 5-formyl-3-methylthiophene-2-boronic acid (18.9 mg, 0.105 mmol) and tetrakis(triphenylphosphine)palladium (22 mg, 0.019 mmol) were dissolved in a solution of DME (10 mL), EtOH (1.6 mL) and saturated aqueous Na₂CO₃ (2.75 mL). The reaction was heated to reflux overnight under an argon atmosphere. The following day the reaction was poured into water, and the aqueous phase extracted three times with CH₂Cl₂. The organic extract was concentrated in vacuo and purified by RP-HPLC (MeCN:H₂O:0.1% TFA). LR-ESI MS (M+H)⁺ m/z calcd 522.2, found 522.0.

### Synthesis of S6

2-((4-amino-3-iodo-1H-pyrazolo[3,4-d]pyrimidin-l-yl)methyl)-5-methyl-3-o-tolylquinazolin-4(3H)-one (100 mg, 0.192 mmol), 3,4-dimethoxyphenyl boronic ester (38.2 mg, 0.21 mmol) and tetrakis(triphenylphosphine)palladium (44 mg, 0.038 mmol) were dissolved in a solution of DME (20 mL), EtOH (3.2 mL) and saturated aqueous Na₂CO₃ (5.5 mL). The reaction was heated to reflux overnight under an argon atmosphere. The following day the reaction was poured into water, and the aqueous phase extracted three times with CH₂Cl₂. The organic extract was concentrated in vacuo and purified by RP-HPLC (MeCN:H₂O:0.1% TFA). LR-ESI MS (M+H)⁺ m/z calcd 534.2, found 534.0.

### Synthesis of S7

2-((4-amino-3-iodo-1H-pyrazolo[3,4-d]pyrimidin-1-yl)methyl)-5-methyl-3-o-tolylquinazolin-4(3H)-one (100 mg, 0.192 mmol), 4-phenoxyphenyl boronic acid (44.9 mg, 0.21 mmol) and tetrakis(triphenylphosphine)palladium (44 mg, 0.038 mmol) were dissolved in a solution of DME (20 mL), EtOH (3.2 mL) and saturated aqueous Na₂CO₃ (5.5 mL). The reaction was heated to reflux overnight under an argon atmosphere. The following day the reaction was poured into water, and the aqueous phase extracted three times with CH₂Cl₂. The organic extract was concentrated in vacuo and purified by RP-HPLC (MeCN:H₂O:0.1% TFA). LR-ESI MS (M+H)⁺ m/z calcd 566.2, found 566.0.

### Synthesis of S8

2-((4-amino-3-iodo-1H-pyrazolo[3,4-d]pyrimidin-1-yl)methyl)-5-methyl-3-o-tolylquinazolin-4(3H)-one (100 mg, 0.192 mmol), 4-benzyloxyphenyl boronic acid (47.9 mg, 0.21 mmol) and tetrakis(triphenylphosphine)palladium (44 mg, 0.038 mmol) were dissolved in a solution of DME (20 mL), EtOH (3.2 mL) and saturated aqueous Na₂CO₃ (5.5 mL). The reaction was heated to reflux overnight under an argon atmosphere. The following day the reaction was poured into water, and the aqueous phase extracted three times with CH₂Cl₂. The organic extract was concentrated in vacuo and purified by RP-HPLC (MeCN:H₂O:0.1% TFA).

### Synthesis of S33

2-((4-amino-3-iodo-1H-pyrazolo[3,4-d]pyrimidin-1-yl)methyl)-5-methyl-3-o-tolylquinazolin-4(3H)-one (50 mg, 0.096 mmol), 3-cyanophenyl boronic acid (15.8 mg, 0.105 mmol) and tetrakis(triphenylphosphine)palladium (22 mg, 0.019 mmol) were dissolved in a solution of DME (10 mL), EtOH (1.6 mL) and saturated aqueous Na₂CO₃ (2.75 mL). The reaction was heated to reflux overnight under an argon atmosphere. The following day the reaction was poured into water, and the aqueous phase extracted three times with CH₂Cl₂. The organic extract was concentrated in vacuo and purified by RP-HPLC (MeCN:H₂O:0.1% TFA). LR-ESI MS (M+H)⁺ m/z calcd 499.2, found 499.0.

### Synthesis of 2-amino-N-(2-chlorophenyl)-6-methylbenzamide

2-amino-6-methylbenzoic acid (2.5 g, 16.5 mmol) was dissolved in benzene (75 mL). Thionyl chloride (3.0 mL, 41.1 mmol) was added and the reaction heated to reflux overnight. The following day the reaction was concentrated in vacuo, and then taken up twice in benzene (75 mL) and solvent removed in vacuo again to give a black oil. The oil was dissolved in CHCl₃ (75 mL), 2-chloroaniline (3.5 mL) was added and the reaction heated to reflux. Reaction was complete after four hours, at which point the reaction was filtered, the filtrate concentrated in vacuo, and the product was purified by silica gel chromatography (25% EtOAc/Hexanes) to yield a brown oil (1.94 g, 45% yield). HR-EI MS (M)⁺ m/z calcd 260.07, found 260.0715.

### Synthesis of 2-(chloromethyl)-3-(2-chlorophenyl)-5-methylquinazolin-4(3H)-one

Chloroacetylchloride (0.72 mL, 9 mmol) was added to a solution of 2-amino-N-(2-chlorophenyl)-6-methylbenzamide (0.8 g, 3.06 mmol) in acetic acid (10 mL) and the reaction heated to reflux. After 2.5 hours the reaction was cooled to RT, and concentrated in vacuo. The product was purified by silica gel chromatography (10% EtOAc/Hexanes) to yield a white solid (0.353 g, 36% yield). HR-EI MS (M)⁺ m/z calcd 318.0327, found 318.0321.

### Synthesis of 2-((4-amino-3-iodo-1H-pyrazolo[3,4-d]pyrimidin-1-yl)methyl)-3-(2-chlorophenyl)-5-methylquinazolin-4(3H)-one

2-(chloromethyl)-3-(2-chlorophenyl)-5-methylquinazolin-4(3H)-one (0.112 g, 0.35 mmol) and 3-iodo-1H-pyrazolo[3,4-d]pyrimidin-4-amine (0.138 g, 0.053 mmol) were added to DMF (5 mL) and K₂CO₃ (0.096 g, 0.7 mmol) and allowed to stir at RT in the dark for 72 hours. The product was precipitated by addition of water (50 mL) and collected by filtration. The precipitate was further purified by RP-HPLC (MeCN:H₂O:0.1% TFA).

### Synthesis of S1

2-((4-amino-3-iodo-1H-pyrazolo[3,4-d]pyrimidin-1-yl)methyl)-3-(2-chlorophenyl)-5-methylquinazolin-4(3H)-one (60 mg, 0.11 mmol), m-phenol boronic acid (17 mg, 0.121 mmol) and tetrakis(triphenylphosphine)palladium (25 mg, 0.022 mmol) were dissolved in a solution of DME (10 mL), EtOH (1.6 mL) and saturated aqueous Na₂CO₃ (2.75 mL). The reaction was heated to reflux overnight under an argon atmosphere. The following day the reaction was poured into water, and the aqueous phase extracted three times with CH₂Cl₂. The organic extract was concentrated in vacuo and purified by RP-HPLC (MeCN:H₂O:0.1% TFA). LR-ESI MS (M+H)⁺ m/z calcd 510.1, found 510.0.

### Synthesis of S34

2-((4-amino-3-iodo-1H-pyrazolo[3,4-d]pyrimidin-l-yl)methyl)-5-methyl-3-o-tolylquinazolin-4(3H)-one (50 mg, 0.096 mmol), benzene 3-sulphonamide boronic ester (29.7 mg, 0.105 mmol) and tetrakis(triphenylphosphine)palladium (22 mg, 0.019 mmol) were dissolved in a solution of DME (10 mL), EtOH (1.6 mL) and saturated aqueous Na₂CO₃ (2.75 mL). The reaction was heated to reflux overnight under an argon atmosphere. The following day the reaction was poured into water, and the aqueous phase extracted three times with CH₂Cl₂. LR-ESI MS (M+H)⁺ m/z calcd 553.2, found 553.0.

### II. Compositions

Pharmaceutical compositions for ocular administration In some embodiments, the invention provides a pharmaceutical composition for ocular administration containing a compound of the present invention, and a pharmaceutical excipient suitable for ocular administration.

In some embodiments, the invention provides a liquid pharmaceutical composition for ocular administration containing: (i) an effective amount of a compound of the present invention; (ii) an effective amount of a second agent; and (iii) a pharmaceutical excipient suitable for ocular administration. In some embodiments, the composition further contains: (iv) an effective amount of a third agent.

Pharmaceutical compositions of the invention suitable for ocular administration can be presented as discrete dosage forms, such as drops or sprays each containing a predetermined amount of an active ingredient a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then if necessary, dissolving or diluting in a liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an ocular dosage form, any of the usual pharmaceutical media can be employed as carriers, such as, for example, water, glycols, oils, alcohols, preservatives, coloring agents, and the like in the case of liquid preparations (such as suspensions, solutions, and elixirs) or aerosols; or carriers such as starches, sugars, micro-crystalline cellulose, diluents, granulating agents, lubricants, binders, and disintegrating agents can be used.

Carriers suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for ocular administration, the essential active ingredient therein may be combined with various coloring matter or dyes and, if so desired, emulsifying and/or suspending agents, together with such diluents as water, ethanol, propylene glycol, glycerin and various combinations thereof.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, preferred ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, .epsilon.-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

In some embodiments, the composition includes an ophthalmic emulsion to increase the bioavailability or intraocular penetrance of the compounds by electrostatic attraction between the emulsions's positive charge and the negative charges carried at the eye surface. Often emulsions must be stabilized to provide a useful shelf-life. One known approach to stabilize an emulsion is to confer an electrostatic charge to the droplets surface which will result in droplet repulsion and less droplet coalescence. Colloidal particles dispersed in a solution are electrically charged due to their ionic characteristics and dipole attributes.

Of particular interest are the following patents dealing with cationic emulsions for topical ocular administration: U.S. Pat. No. 6,007,826 discloses a cationic oil-in-water emulsion which comprises colloid particles with a positively charged interfacial film. The interfacial film is formed by cationic lipids (0.05-3% by weight) such as C10-C14 primary alkylamines (disclosed are stearylamine or oleylamine), C10-C24 primary alkanolamine or a cholesterol betainate; phospholipids (0.5-3%) and non-ionic surfactants from the group consisting of poloxamers, tyloxapol, polysorbate, and polyoxyethylene fatty acid esters (0.05-3%). The concentration of the oily core is maintained within the 3-20% range. U.S. patent application Ser. No. 12/089,609 discloses a cationic oil-in-water emulsion which provides enhanced stability of the drug compounds emulsified therein.

It is generally accepted that in order to show good ocular tolerability the cation content within the formulation should not exceed 0.1%, preferably not exceed 0.05% and even more preferably should not exceed 0.03%. Quaternary amines such as benzalkonium chloride, benzododecinium bromide and benzethonium chloride are allowed by health authorities for ophthalmic administration up to concentration of approximately 0.03% (Furrer et al., Eur. J. Pharm. Biopharm. 2002, 53:263-280).

In some embodiments cationic-oil-in-water emulsions comprise one or more PI3K antagonists or mTORC1/mTORC2 inhibitors, at least one cationic agent and at least one non ionic surfactant selected from selected from the group consisting of poloxamers, tyloxapol, polysorbates, polyoxyethylene castor oil derivatives, sorbitan esters, polyoxyl stearates and a mixture of two or more thereof.

According to a specific embodiment of the invention, the emulsion may further comprise an anti-inflammatory compound, preferably a non steroidal anti-inflammatory compound or a omega-3 fatty acid. Anti-inflammatory agents may be chosen in the group comprising COX-2 inhibitors, salicylates, 2-arylpropionic acids, N-arylanthranilic acids, oxicams, sulphonanilides, pyrazolidines derivatives, arylalkanoic acids, 3-benzolphenylacetic acids and derivatives; steroids such as cortisone, hydrocortisone, prednisone, prednisolone, methylprednisone, fluoromethalone, medrysone, betamethasone, loteprednol, flumethasone, mometasone, testosterone, methyltestosterone, danazol, beclomethasone, dexamethasone, dexamethasone palmitate, tramcinolone, triamcinolone acetonide, fluocinolone, fluocinolone acetonide, difluprednate, rimexolone.

According to an embodiment of the invention, the oil phase comprises one or more components selected from the group comprising or consisting of mineral oil such as for example petrolatum and liquid paraffin, and light mineral oil, medium chain triglycerides (MCT) which is generally defined as a triglyceride oil in which the carbohydrate chain has about 8-12 carbon atoms, coconut oil; hydrogenated oils comprising hydrogenated cottonseed oil, hydrogenated palm oil, hydrogenate castor oil or hydrogenated soybean oil; polyoxyethylene hydrogenated castor oil derivatives comprising poluoxyl-40 hydrogenated castor oil, polyoxyl-60 hydrogenated castor oil or polyoxyl-100 hydrogenated castor oil.

### III. Methods

### Methods of Treating Ophthalmic Diseases

In one embodiment, the present invention provides methods and compositions for treating diseases and conditions related to angiogenesis, especially angiogenesis mediated by ocular cells or taking place inside a subject's eye. Angiogenesis can be regulated by signal transduction pathways which include a variety of signaling molecules such as kinases. Kinases can generally be classified into protein kinases and lipid kinases, and certain kinases exhibit dual specificities. Protein kinases are enzymes that phosphorylate other proteins and/or themselves (i.e., autophosphorylation). Protein kinases can be generally classified into three major groups based upon their substrate utilization: tyrosine kinases which predominantly phosphorylate substrates on tyrosine residues (e.g., erb2, PDGF receptor, EGF receptor, VEGF receptor, src, abl), serine/threonine kinases which predominantly phosphorylate substrates on serine and/or threonine residues (e.g., mTorC 1, mTorC2, ATM, ATR, DNA-PK, Akt), and dual-specificity kinases which phosphorylate substrates on tyrosine, serine and/or threonine residues.

Lipid kinases are enzymes that catalyze the phosphorylation of lipids within cells. These enzymes, and the resulting phosphorylated lipids and lipid derived biologically active organic molecules, play a role in many different physiological processes, including cell proliferation, migration, adhesion, and differentiation. A particular group of lipid kinases comprises membrane lipid kinases, i.e., kinases that catalyze the phosphorylation of lipids contained in or associated with cell membranes. Examples of such enzymes include phosphinositide(s) kinases (such as PI3-kinases, PI4-Kinases), diacylglycerol kinases, and sphingosine kinases.

The phosphoinositide 3-kinases (PI3Ks) signaling pathway is one of the most highly mutated systems in human cancers. PI3Ks are members of a unique and conserved family of intracellular lipid kinases that phosphorylate the 3′-OH group on phosphatidylinositols or phosphoinositides. The PI3K family comprises 15 kinases with distinct substrate specificities, expression patterns, and modes of regulation (Katso et al., 2001). The class I PI3Ks (p110α, p110β, p110δ, and p110γ) are typically activated by tyrosine kinases such as VEGF-R or G-protein coupled receptors to generate PIP3, which engages downstream effectors such as the Akt/PDK1 pathway, mTOR, the Tec family kinases, and the Rho family GTPases. The class II and III PI3-Ks play a key role in intracellular trafficking through the synthesis of PI(3)P and PI(3,4)P2. The PIKKs are protein kinases that control cell growth (mTORC1) or monitor genomic integrity (ATM, ATR, DNA-PK, and hSmg-1).

Downstream mediators of the PI3K signal transduction pathway include Akt and mammalian target of rapamycin (mTOR). Akt posseses a pleckstrin homology (PH) domain that binds PIP3, leading to Akt kinase activation. Akt phosphorylates many substrates and is a central downstream effector of PI3K for diverse cellular responses. Full activation of Akt typically requires phosphorylation of T308 in the activation loop and S473 in a hydrophobic motif. One important function of Akt is to augment the activity of mTOR, through phosphorylation of TSC2 and other mechanisms. mTOR is a serine-threonine kinase related to the lipid kinases of the PI3K family. mTOR has been implicated in a wide range of biological processes including cell growth, cell proliferation, cell motility and survival. Disregulation of the mTOR pathway has been reported in various types of cancer. mTOR is a multifunctional kinase that integrates growth factor and nutrient signals to regulate protein translation, nutrient uptake, autophagy, and mitrochondrial function.

Dysregulation of signaling pathways mediated by many kinases is a key factor in the development of human diseases. Aberrant or excessive protein kinase activity or expression has been observed in many disease states including ocular diseases and conditions, immune disorders, benign and malignant proliferative diseases, disorders such as allergic contact dermatitis, rheumatoid arthritis, osteoarthritis, inflammatory bowel diseases, chronic obstructive pulmonary disorder, psoriasis, multiple sclerosis, asthma, disorders related to diabetic complications, and inflammatory complications of the cardiovascular system such as acute coronary syndrome.

In one embodiment of the present invention, methods are provided for treating diseases and conditions related to aberrant growth and/or permeability of the vasculature of the eye. Diseases and conditions related to aberrant growth and/or permeability of the vasculature of the eye include but are not limited to age-related macular degeneration, retinopathy of prematurity, ischemic retinal vein occlusion, diabetic retinopathy, and neovascular glaucoma.

In some embodiments, methods for treating aberrant growth and/or permeability of the vasculature of the eye include the use of inhibitors of the vascular endothelial growth factor (VEGF) signal transduction pathway and/or the hypoxia inducible factor (HIF) signal transduction pathway. In some cases, said methods include the use of, or administration of, antagonists (e.g. inhibitors) of PI3K, which is an important downstream mediator of VEGF and HIF-α signaling. PI3K antagonists of the present invention include compounds that are specific for a single class of PI3K such as PI3Kα, PI3Kβ, PI3Kγ, or PI3Kδ. Compounds of the present invention that specifically inhibit a single class of PI3K may in some cases provide enhanced treatment modalities by leading to a lower number, severity, and/or frequency of side-effects. In some embodiments of the present invention antagonists are administered that are specific for a single class of PI3K, for example, they inhibit one class of PI3K with an IC50 that is at least 2-fold lower than for another class of PI3K. In other cases, specific PI3K antagonists of the present invention may be administered that inhibit a first class of PI3K such as, for example, PI3Kδ with a IC50 of less than about 1, 5, 10, 50, 100, 150, 200, 300, 400, 500, 750, or less than about 1000 nM, while at that antagonist concentration exhibiting less than about 1%, 2%, 3%, 4%, 5%, 10%, 20%, 25%, 30%, 35%, or less than about 40% inhibition of another class of PI3K. In some embodiments, PI3Kδ antagonists are administered to the eye of a subject to inhibit inflammatory responses that lead to activation of the VEGF signaling pathway.

In some cases, compounds of the present invention are administered and they are specific for more than one class of PI3K. For example, PI3K antagonists of the present invention may substantially and specifically inhibit classes I, II, or III of PI3K. As used herein, the term “pan-specific PI3K inhibitors” refers to PI3K antagonist compounds of the present invention that inhibit two or more classes or isoforms of PI3Ks. The administration of antagonists of different classes of PI3Ks provides the ability to inhibit angiogenesis at multiple points in the signal transduction pathway. Such multi-point inhibition may in some cases provide additive or even synergistic effects as compared to the administration of compounds that are directed to a single isoform.

In some embodiments, the administered compounds may selectively inhibit one or more members of type I or class I phosphatidylinositol 3-kinases (PI3-kinase) with an IC50 value of about 100 nM, 50 nM, 10 nM, 5 nM, 100 pM, 10 pM or even 1 pM, or less as ascertained in an in vitro kinase assay.

In other embodiments, the administered compounds may selectively inhibit one or two members of type I or class I phosphatidylinositol 3 -kinases (PI3-kinase) consisting of PI3-kinase α, PI3-kinase β, PI3-kinase γ, and PI3-kinase δ. In some aspects, some of the subject compounds selectively inhibit PI3-kinase δ as compared to all other type I PI3-kinases. In some aspects, the administered compounds selectively inhibit PI3-kinase α as compared to all other type I PI3-kinases. In some aspects, the administered compounds selectively inhibit PI3-kinase γ as compared to all other type I PI3-kinases. In other aspects, some of the subject compounds selectively inhibit PI3-kinase δ and PI3-kinase y as compared to the rest of the type I PI3-kinases. In yet other aspects, some of the subject compounds selectively inhibit PI3-kinase a and PI3-kinase β as compared to the rest of the type I PI3-kinases. In still yet some other aspects, some of the subject compounds selectively inhibit PI3-kinase δ and PI3-kinase β as compared to the rest of the type I PI3-kinases.

In yet another aspect, an inhibitor selectively inhibits one or more members of type I PI3-kinase inhibitor, or an inhibitor that selectively inhibits one or more members of the type I PI3-kinases mediated signaling, alternatively can be understood to refer to a compound that exhibits a 50% inhibitory concentration (IC₅₀) with respect to a given type I PI3-kinase, that is at least at least 10-fold, at least 20-fold, at least 50-fold, at least 100-fold, at least 1000-fold, at least 10,100-fold, or lower, than the inhibitor's IC₅₀ with respect to the rest of the other type I PI3-kinases.

By way of example only, compounds that antagonize PI3Kδ and PI3Kγ can inhibit inflammatory responses that lead to or exacerbate aberrant neovascularization and/or vascular permeability. Agonist against PI3Kα can have a direct inhibitory effect on angiogenesis. The use of antagonists that inhibit multiple isoforms of class I PI3K provides methods for inhibiting signal transduction pathways that lead to neovascularization and/or vascular permeability at multiple points along the pathway. Similarly, in some cases, the compounds of the present invention that are administered inhibit kinases other than PI3Ks including but not limited to mTOR, Akt, Src, MAPK, MEK, Jun Kinases, receptor tyrosine kinases such as VEGF-R, DNA-PK, Abl, Hck, EGFR, EphB4, and PDGFR. In some embodiments, methods for administering a combination of compounds of the present invention are provided that inhibit VEGF signaling at several points such as but not limited to PI3K signaling, VEGF-R signaling, GPCR signaling, and inflammatory-mediated vascular permeability and neovascularization.

In some embodiments methods are provided for the administration of inhibitors of downstream mediators of the VEGF signaling pathway such as PI3K inhibitors to modulate VEGF (VEGF) signaling in cells of ocular origin. Cells of ocular origin include but are not limited to extrafoveal cells, subfoveal cells, juxtafoveal cells, scleral cells, conjunctival cells, retinal cells, retinal pigmented epithelial cells, lens cells, muscle cells, neurons, rod cells, cone cells, cells of the cornea, cells of the pupil, cells of the iris, cells of the optic nerve, cells of the choroidal layer, and vascular epithelial cells within and surrounding the eye. In some cases, cells of ocular origin may be assayed in vitro to determine the effect of the methods and compounds of the present invention on VEGF signaling, in other cases, cells of ocular origin are assayed in vivo to determine the effect of the methods and compounds of the present invention on VEGF signaling.

Methods for assaying the effect of PI3K or mTOR inhibitors on VEGF signaling are well known in the art and include, but are not limited to, the use of angiogenesis assays such as described in Auerbach, R. et al. Clinical Chemistry. 49:1, 32-40; the use of VEG-F signaling assays such as the HTScan® VEGF Receptor 3 Kinase Assay Kit from cell signaling technologies; cell permeability assays; the assays described in US Patent Application Publication No. 2003/020,208; the cornea micropocket angiogenesis assay. In some embodiments, modulation of VEGF signaling or reducing angiogenesis is assayed by measuring kinase activity in vitro or in vivo by the use of anti-phospho protein specific antibodies such as, but not limited to, anti-phospho VEGF-R, anti-phsopho Akt, anti-phospho S6, anti-phospho PRAS40, and anti-phospho 4EBP; anti-phospho RET, anti-phospho FOXO3A, anti-phospho FOXO1, and anti-phospho GSK3b. In some embodiments, inhibition of VEGF and/or HIF signaling is indicated by the successful inhibition of aberrant angiogenesis and/or vascular permeability in a subject suffering from an eye disease or condition.

In another embodiment of the present invention, methods are provided for modulating, mitigating, or treating an ophthalmic disease by administering one or more compounds that inhibit both mTORC 1 and mTORC2. In some cases, the compounds are administered to the eye; and in other cases, the compounds are administered systemically. In some embodiments, some the compounds of the present invention to be administered inhibit both mTORC1 and mTORC2. In some cases, the compounds inhibit mTORC1 and mTORC2 with an IC50 of between less than about 1nM to less than about 1 μM, including about 1nM, 2nM, 4 nM, 8 nM, 10 nM, 15 nM, 20 nM, 40 nM, 80 nM, 100 nM, 150 nM, 200 nM, 250 nM, 500 nM, 750 nM, and about 1000 nM. Methods for determining the level of kinase inhibition are provided herein and include for example in vitro kinase assays, in vivo kinase assays, and cell proliferation assays. In some cases, inhibition of mTORC1 and mTORC2 is determined by measuring Akt phosphorylation. In some cases, the administration of mTORC1/mTORC2 inhibitors antagonizes or inhibits the VEGF and/or HIF signaling pathway. In some cases, the antagonists of mTOR reduce angiogenesis in the eye or vascular permeability in cells or tissues of ocular origin.

In another embodiment of the present invention, methods are provided for inhibiting angiogenesis in the eye by administering a combination of an mTORC1/mTORC2 inhibitor and one or more PI3K antagonists. In some cases the combination of mTORC1/mTORC2 inhibitor and one or more PI3K antagonists inhibits mTORC1/mTORC2 and one or more of PI3Kα, PI3KIβ, PI3Kγ, or PI3K6 with an IC50 of less than about 1 μM. In some cases, the combination inhibits mTORC1/mTORC2 and one or more of PI3Kα, PI3KIβ, PI3Kγ, or PI3Kδ with an IC50 of less than about 750 nM, 500 nM, 350 nM, 250 nM, 200 nM, 150 nM, 100 nM, 50 nM, 25 nM, 10 nM, 5 nM, or less than about 1 nM.

In another embodiment of the present invention, methods are provided for inhibiting the inflammatory response in the eye mediated by white blood cells such as but not limited to macrophages, neutrophils, and microglia. White blood cell induced inflammatory responses have been implicated in such ocular diseases and conditions as, for example, sarcoidosis, sympathetic ophthalmia, Vogt-Koyanagi-Harada disease, age-related macular degeneration, uveitis, and other ocular diseases mediated by inflammation, vascular permeability, and/or angiogenesis.

In some cases, white blood cell induced inflammatory responses lead to aberrant neovascularization and/or vascular permeability due to activation of the VEGF signaling pathway. Accordingly, the inflammatory response mediated by white blood cells may in some cases be inhibited by reducing white blood cell activation (e.g. proliferation and/or cytokine release), or by inhibiting the VEGF signaling pathway. In some cases, methods are provided for administration of the compounds of the present invention to the eye such that B-cell activation, leukocyte activation, macrophage activation, neutrophil activation, or microglial cell activation is inhibited. In some cases, the compounds administered inhibit the activation or proliferation of one or more types of white blood cell with an IC50 of less than about 250 nM, including less than about 1 nM, 5 nM, 10 nM, 25 nM, 50 nM, 100 nM, 150 nM, 200 nM, and less than about 250 nM.

Methods for assaying B-cell proliferation include the method described by Hashimoto et al. J Immunol Methods. 1986 Jun. 10;90(1):97-103. Hashimoto et al. describe a method for rapid colorimetric determination of alkaline phosphatase (APase) activity that can be used in conjunction with a multiwell scanning photometer to quantitatively measure lymphokine-dependent B cell proliferation. Other methods for B-cell proliferation assays include Storek et al. J Immunol Methods. 1992 Jul. 6;151(1-2):261-7. Storek et al. describe a simple assay consisting of identifying the cultured B cells with anti-CD19/CD20 fluorescein-conjugated antibody and simultaneously using propidium iodide DNA staining to determine the percent of S/G2/M phase cells among the gated B cells. Still other methods for assaying lymphocyte proliferation include tritiated thymidine incorporation, BrDu incorporation, or EdU incorporation.

Methods for assaying neutrophil activation include those described in Lieberman et al. CLINICAL AND DIAGNOSTIC LABORATORY IMMUNOLOGY, November 1996, p. 654-662. Lieberman et al. describe two methods for assaying neutrophil activation. In the first method, neutrophils activation is measured by assaying the cellular debris fraction of neutrophil lysate in a microtiter plate for NADPH oxidase activity using luminol as a substrate. In the second method, the uptake and oxidation of the non-fluorescent dye dihydrorhodamine 123 to the fluorescent rhodamine 123 by activated neutrophils is monitored by flow cytometry.

In some embodiments, white blood cell activation may be measured using any one of a number of general techniques known in the art for assaying cell proliferation. Such techniques include the MTT assay, the XTT assay, and measuring uptake of BrdU or an analogue thereof such as EdU, and tritiated thymidine incorporation assays. In some embodiments, white blood cell activation in vivo may be measured by administration of the compounds of the present invention, such as for example daily for about two weeks and harvesting white blood cells and/or lymphoid tissues for analysis. In some embodiments, a reagent (e.g. BrDu or EdU) for detecting proliferation is administered approximately 2 hours before harvesting to assay for lymphocyte proliferation.

### Methods of Administration

The compositions for use in the methods of the invention may be administered via any viable delivery method or route, however in some cases, local administration is preferred. It is contemplated that all local routes to the eye may be used including topical, subconjunctival, periocular, retrobulbar, subtenon, intracameral, intravitreal, intraocular, subretinal, juxtascleral and suprachoroidal administration. Systemic or parenteral administration may be feasible including but not limited to intravenous, subcutaneous, and oral delivery. The most preferred method of administration will be intravitreal or subtenon injection of solutions or suspensions, or intravitreal or subtenon placement of bioerodible or non-bioerodible devices, or by topical ocular administration of solutions or suspensions, or posterior juxtascleral administration of a gel or cream formulation.

For topical administration, all the formulations for topical ocular administration used in the field of ophthalmology (e.g., eye drops, inserts, eye packs, impregnated contact lenses, pump delivery systems, dimethylsulfoxide (DMSO)-based solutions suspensions, liposomes, and eye ointment) and all the formulations for external use in the fields of dermatology and otolaryngology (e.g., ointment, cream, gel, powder, salve, lotion, crystalline forms, foam, and spray) may be utilized as is known in the art.

Eye drops may be prepared by dissolving the active ingredient in a sterile aqueous solution such as physiological saline, buffering solution, etc., or by combining powder compositions to be dissolved before use. Other vehicles may be chosen, as is known in the art, including but not limited to: balance salt solution, saline solution, water soluble polyethers such as polyethyene glycol, polyvinyls, such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate. If desired, additives ordinarily used in the eye drops can be added. Such additives include isotonizing agents (e.g., sodium chloride, etc.), buffer agent (e.g., boric acid, sodium monohydrogen phosphate, sodium dihydrogen phosphate, etc.), preservatives (e.g., benzalkonium chloride, benzethonium chloride, chlorobutanol, etc.), thickeners (e.g., saccharide such as lactose, mannitol, maltose, etc.; e.g., hyaluronic acid or its salt such as sodium hyaluronate, potassium hyaluronate, etc.; e.g., mucopolysaccharide such as chondroitin sulfate, etc.; e.g., sodium polyacrylate, carboxyvinyl polymer, crosslinked polyacrylate, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents known to those skilled in the art).

Another preferred method of delivery is intravitreal administration of a bioerodible implant administered through a device such as that described in U.S. application Ser. No. 60/710,046, filed Aug. 22, 2005. Another preferred method of delivery is with a microfluidic implant device such as that described in U.S. Pat. No. 6,976,982. Additionally, the pharmaceutical compositions of the present invention may be administered to the ocular surface via a pump-catheter system, or released from within a continuous or selective release device such as, e.g., membranes such as, but not limited to, those employed in the Ocusert™ System (Alza Corp, Palo Alto, Calif.). The pharmaceutical compositions can be incorporated within, carried by or attached to contact lenses which are then worn by the subject. The pharmaceutical compositions can be sprayed onto ocular surface.

It is envisioned additionally, that the compounds of the invention may be attached releasably to biocompatible polymers for use in sustained release formulations on, in or attached to inserts for topical or systemic administration. The controlled release from a biocompatible polymer may be utilized with a water soluble polymer to form a instillable formulation, as well. Ocular conditions, diseases and disorders, may in some cases be treated by introducing slow release drug-containing microcapsules or implants directly into the anterior and/or posterior chambers of the eye. The microcapsules may be formulated to include one or more drugs which may be released over an extended period of time at a therapeutically effective dosage into the vitreous humor. In this manner, drug released from microcapsules placed into the anterior chamber will reach the cornea, aqueous humor, trabecular mesh work, iris, lens, and related structures in the anterior chamber. Microcapsules introduced into the posterior chamber are diffused throughout the vitreous in the chamber and into the entire retina (which consists of 10 different layers), into the choroid and the apposed sclera. Thus, the drug will be available at the site(s) where the drug is needed and will be maintained at an effective dosage.

In some cases, the primary element of the capsule may be the polymeric encapsulating agent or lipid encapsulating agent. The compositions may further be biocompatible, and in some cases biodegradable. In some embodiments, the most part, the polymeric compositions may be organic esters or ethers, which when degraded result in physiologically acceptable degradation products, including the monomers. However, anhydrides, amides, orthoesters or the like, by themselves or in combination with other monomers may also find use. The polymers may be addition or condensation polymers, particularly condensation polymers. The polymers may be cross-linked or non-cross-linked, usually not more than lightly cross-linked, generally less than 5%, usually less than 1%. For the most part, besides carbon and hydrogen, the polymers will include oxygen and nitrogen, particularly oxygen. The oxygen may be present as oxy, e.g. hydroxy or ether, carbonyl, e.g. non-oxo-carbonyl, such as carboxylic acid ester, and the like. The nitrogen may be present as amide, cynao and amino.

In some embodiments, the drug eluting devices may be made of biodegradable polymers such as polyglycolide, polylactide, poly ε-caprolactone, polyglyconate, polyhydroxybutyrate, polyhydroxyvalerate, and polydioxanone polymers. These biodegradable polymers as well as others described herein provide for slow release of compounds as the device degrades in vivo.

In some embodiments drug eluting devices may be made using polymers of hydroxyaliphatic carboxylic acids, either homo- or copolymers, and polysaccharides, Included among the polyesters of interest are polymers of D-lactic acid, L-lactic acid, racemic lactic acid, glycolic acid, polycaprolactone, and combinations thereof. In some cases, by employing the L-lactate, a slowly eroding polymer may be achieved, while erosion may be substantially enhanced with the lactate racemate.

In other embodiments, the drug eluting device may include a polysaccharides such as for example calcium alginate, or functionalized celluloses, particularly carboxymethylecellulose esters characterized by being water insoluble, with a molecular weight of about 5 kD to 500 kD, etc. Other polymers of interest include polyvinyl alcohol, esters and ethers, which are biocompatible and may be biodegradable. In some cases, characteristics of the polymers include biocompatibility, compatibility with the drug, ease of encapsulation, a halflife in the physiological environment of at least 6 hrs, preferably greater than one day, polymers that do not significantly enhance the viscosity of the vitreous, water insoluble, and the like.

In other embodiments, the drug eluting device may include a protein polymer such as for example collagen, matrigel, gelatin or other large molecular weight protein. Protein polymers have the advantage of being in some cases particularly biocompatible because they are derived from natural sources.

The biodegradable polymers which form the microencapsulated particles may in some cases be subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, where the polymers may be employed as varying layers or mixed.

By employing a biodegradable polymer, particularly one where the biodegradation is relatively slow, the rate of release of the drug will be primarily diffusion controlled, depending upon the surrounding membrane or monolithic polymer structure, rather than breakdown of the particle. For the most part, the selected particles will have lifetimes at least equal to the desired period of administration, preferably at least twice the desired period of administration, and may have lifetimes of 5 to 10 times the desired period of administration. The period of administration may be at least 3 days, more usually at least 7 days, generally at least about 15 days and may be 20 days or more.

In other embodiments, the drug eluting device may be made up of non-biodegradable polymers or constituents including but not limited to silicone, acrylates, polyethylenes, polyurethane, polyester, polypropylene, polytetrafluoroethylene, poly ether ketone, nylon, collagen, polyethylene terepthalate, polycarbonate, and polyimide polymers, or non-ferrous metals including biocompatible allows of steel and titanium. Non-biodegradable devices may be constructed to contain a drug containing reservoir and one or more pores for release of therapeutic agents. In other cases, non-biodegradable devices are swelled with a solution of therapeutic agent which is then released upon implantation. In still other cases, non-biodegradable devices may be coated with a biodegradable or other coating containing therapeutic agent. The coating may further possess suitable characteristics for slow release of the therapeutic compound contained therein. For example, the coating may be biodegradable or bioerodable, or the coating may contain a plurality of micropores, or the coating may have an reversible affinity for the therapeutic agent such that the therapeutic agent is slowly released at equillibrium.

The particles may be substantially homogeneous as to composition and physical characterstics or heterogeneous. Thus, particles can be prepared where the center may be of one material and the surface have one or more layers of the same or different composition, where the layers may be cross-linked, of different molecular weight, different density or porosity, or the like. For example, the center could be a polylactate coated with a polylactate-polyglycolate copolymer, so as to enhance the rate of initial degradation. Most ratios of lactate to glycolate employed will be in the range of about 1:0-1. Alternatively, the center could be polyvinyl alcohol coated with polylactate, so that on degradation of the polylactate the center would dissolve and be rapidly washed out of the eye.

Any pharmacologically active agent for which sustained release is desirable may be employed. Desirably, the drug will be sufficiently soluble in the vitreous to be presented at a pharmacologically effective dose. Pharmacologic agents which may find use include the compounds of the present invention as well as those found in U.S. Pat. Nos. 4,474,451, columns 4-6 and 4,327,725, columns 7-8, which disclosures are incorporated herein by reference in their entirety.

Drugs of particular interest include the compounds of the present invention as well as hydrocortisone (5-20 mcg/l as plasma level), gentamycin (6-10 mcg/ml in serum), 5-fluorouracil (.about.30 mg/kg body weight in serum), sorbinil, IL-2, TNF, Phakan-a (a component of glutathione), thioloa-thiopronin, Bendazac, acetylsalicylic acid, trifluorothymidine, interferon (α, β and γ), immune modulators, e.g. lymphokines, monokines, and growth factors, etc.

Other drugs of interest include anti-glaucoma drugs, such as the beta-blockers: timolol maleate, betaxolol and metipranolol; mitotics: pilocarpine, acetylcholine chloride, isofluorophate, demacarium bromide, echothiophate iodide, phospholine iodide, carbachol, and physostigimine; epinephrine and salts, such as dipivefrin hydrochloride; and dichlorphenamide, acetazolamide and methazolamide; anti-cataract and anti-diabetic retinopathy drugs, such as aldose reductase inhibitors: tolrestat, lisinopril, enalapril, and statil; thiol cross-linking drugs other than those considered previously; anti-cancer drugs, such as retinoic acid, methotrexate, adriamycin, bleomycin, triamcinoline, mitomycin, cis-platinum, vincristine, vinblastine, actinomycin-D, ara-c, bisantrene, CCNU, activated cytoxan, DTIC, HMM, melphalan, mithramycin, procarbazine, VM26, VP16, and tamoxifen; immune modulators, other than those indicated previously; anti-clotting agents, such as tissue plasminogen activator, urokinase, and streptokinase; anti-tissue damage agents, such as superoxide dismutase; proteins and nucleic acids, such as mono- and polyclonal antibodies, enyzmes, protein hormones and genes, gene fragments and plasmids; steriods, particularly antiinflammatory or anti-fibrous drugs, such as cortisone, hydrocortisone, prednisolone, prednisome, dexamethasone, peogesterone-like compounds, medrysone (HMS) and fluorometholone; non-steroidal anti-inflammatory drugs, such as ketrolac tromethamine, dichlofenac sodium and suprofen; antibiotics, such as loridine (cephaloridine), chloramphenicol, clindamycin, amikacin, tobramycin, methicillin, lincomycin, oxycillin, penicillin, amphotericin B, polymyxin B, cephalosporin family, ampicillin, bacitracin, carbenicillin, cepholothin, colistin, erythromycin, streptomycin, neomycin, sulfacetamide, vancomycin, silver nitrate, sulfisoxazole diolamine, and tetracycline; other antipathogens, including anti-viral agents, such as idoxuridine, trifluorouridine, vidarabine (adenine arabinoside), acyclovir (acycloguanosine), pyrimethamine, trisulfapyrimidine-2, clindamycin, nystatin, flucytosine, natamycin, miconazole and piperazie derivatives, e.g. diethylcarbamazine; cycloplegic and mydriatic agents, such as atropine, cyclogel, scopolamine, homatropine and mydriacyl.

Other agents include anticholinergics, anticoagulants, antifibrinolytic agents, antihistamines, antimalarials, antitoxins, chelating agents, hormones, immunosuppressives, thrombolytic agents, vitamines, salts, desensitizing agents, prostaglandins, amino acids, metabolites and antiallergenics.

The amount of drug employed in the device may vary widely depending on the effective dosage required and rate of release. Usually the drug may be from about 1 to 80, more usually 20 to 40 weight percent of the microcapsule.

Other agents may be employed in the formulation for a variety of purposes. In addition to the drug agent, buffering agents and preservatives may be employed. Water soluble preservatives include sodium bisulfate, sodium thiosulfate, ascorbate, benzalkonium chloride, chlorobutanol, thimerosal, phenylmercuric borate, parabens, benzyl alcohol and phenylethanol. These agents may be present in amounts of from 0.001 to 5% by weight and preferably 0.01 to 2%. Suitable water soluble buffering agents are alkali or alkaline earth, carbonates, phosphates, bicarbonates, citrates, borates, acetates, succinates and the like, such as sodium phosphate, citrate, borate, acetate, bicarbonate and carbonate. These agents may be present in amounts sufficient to maintain a pH of the system of between 2 to 9 and preferably 4 to 8. As such the buffering agent may be as much as 5% on a weight to weight basis of the total composition.

The particles may be of a narrow or broad range in size, normally not exceeding 300 μm, so as to be capable of being administered with an 18 gauge needle. Usually, the particle range will not differ by greater than about 200% of the average particle size, more usually not greater than about 100%. The average particle size will usually be in the range of 5 μm to 2 mm, more usually in the range of 10 μm to 1 mm. In some instances the particles will be selected to have an average diameter in the range of 1-2 mm to provide large depots, while in other instances the particles will have average diameter in the range of about 25-500 μm, to provide smaller depots. The size of the particle can be used to control the rate of release, period of treatment and drug concentration in the eye. In some situations mixtures of particles may be employed employing the same or different pharmacological agent. In this way in a single administration a course of drug treatment may be achieved, where the pattern of release may be greatly varied.

Various techniques may be employed to produce the encapsulated drugs. Useful techniques include solvent-evaporation methods, phase separation methods, interfacial methods and the like.

In preparing the encapsulated drugs, in some embodiments solvent-evaporation methods may be employed. Towards this end, the preformed rate controlling polymer may be dissolved in a volatile substantially water-immiscible solvent, such as chloroform, methylene chloride, or benzene. Sometimes, the water immiscible sovlent may be modified with a small amount of a water-miscible organic cosolvent, particularly an oxygenated solvent, such as acetone, methanol, ethanol, etc. Usually, the water-miscible organic cosolvent will be less than about 40 vol %, usually less than about 25 vol %. The drug may then be added to the polymer-solvent solution. Depending upon the nature of the drug, one may have the drug dispersed in the viscous polymer-solvent mixture or a solid dispersion of drug particles, where the drug may in some cases have been pulverized to obtain a fine powder, sometimes a microfine powder particularly of a size of less than about 1 mm, usually less than about 0.5 mm, and may be about 0.5 μm or smaller.

The amount of polymer employed in the medium may vary with the size of the particle desired, whether additional coatings will be added, the viscosity of the solution, the solubility of the polymer and the like. Usually, the concentration of polymer may be in the range of 10 to 80 weight percent. The ratio of drug to polymer may vary with the desired rate of release, the amount of drug generally varying in the range of 1 to 80 weight percent of the polymer.

The dispersion or solution obtained above may be added to a rapidly stirred aqueous solution comprising water and a dispersing agent, which may be a protective colloid. Of particular interest as macromolecular dispersing agents are agents such as poly(vinyl alcohol) (1-5%) or non-ionic detergents, such as Span detergents.

The volume of the organic phase may be smaller than the aqueous phase, generally being in a volume ratio of from about 1:1 to 10:3 of organic to aqueous phase, and an oil-in-water emulsion is produced. The rate of stirring may selected to produce the appropriate droplet size and stirring may be continued throughout the next step.

In the third step, the microencapsulation vessel may be closed and a mild vacuum is applied to the system to evaporate the volatile organic solvent. The solvent may be evaporated slowly, since too rapid evaporation results in bubbles and blow holes formed in the microcapsule walls. The rate of evaporation may be determined empirically, using the experience reported in the literature. Usually the vacuum may be in the range of about 3 to 10 mm Hg. After evaporation has been completed, the resulting microcapsules can be centrifuged, washed completely with water, collected, e.g., filtration, and drained. Usually, the microcapsules may then be subdivided with sieves to isolate particles of a size range of the desired diameter.

The process may be carried out conveniently at room temperature, but cooling or heating may be employed in specific situations to optimize the process. The ratio of drug to polymer may adjusted to produce optimized compositions, since the final product will normally result in the initial ratio. By manipulating the initial bulk viscosity of the drug-polymer-solvent mixture and of the aqueous dispersing medium, along with the stir rate, production of microcapsules with the desired size may be optimized. Moreover, the composition of dissolved organic solvent and the rate of solvent evaporation can be tested to produce microcapsules with larger or smaller crystals of drug in the microcapsules. For polymers which are hydrolytically sensitive, the microcapsules should not be exposed to the aqueous dispersing medium for excessively long periods during the solvent-evaporation step.

The particle size distribution of each batch of microcapsules may be relatively narrow. However, when desired, the size-fractions may be further refined by a physical separation process such as dry or wet sieving.

In order to define the potential drug-release behavior of the microcapsules in vivo, a weighed sample of microcapsules may be added to a measured volume of a solution containing four parts by weight of ethanol and six parts by weight of deionized water. The mixture may be maintained at 37° C. and stirred slowly to maintain the microcapsules suspended. The appearance of the dissolved drug as a function of time may be followed spectrophotometrically until the absorbance becomes constant or until greater than 90% of the drug has been released. The drug concentration after 1 h in the medium is indicative of the amount of free unencapsulated drug in the dose, while the time required for 90% drug to be released is related to the expected duration of action of the dose in vivo. As a general rule, one day of drug release in vitro is approximately equal to 35 days of release in vivo. While release may not be uniform, preferably the release will be free of larger fluctuations from some average value which allows for a relatively uniform release.

The microcapsules may be administered into the eye in a variety of ways, including injection, infusion, trocar, etc. Various techniques for introducing materials into the anterior and/or posterior chambers are well known, see, for example, Liu et al., 1987, supra, and references cited therein.

In general, the doses of the active agents in the compositions used for the above described purposes will vary, but will be in effective amounts to inhibit or cause regression of neovascularization or angiogenesis and to provide neuroprotection to the retinal tissues. In general, the doses of the one or more kinase inhibitor in the compositions of the invention will be in an effective amount to treat or prevent the progression of AMD, DR, sequela associated with retinal ischemia, and macular and/or retinal edema. As used herein, the term “pharmaceutically effective amount” refers to an amount of one or more kinase inhibitor which will effectively treat AMD, DR, and/or retinal edema, or inhibit or cause regression of neovascularization or angiogenesis, in a human patient. The doses used for any of the above-described purposes will generally be from about 0.01 to about 100 milligrams per kilogram of body weight (mg/kg), administered one to four times per day. When the compositions are dosed topically, they will generally be in a concentration range of from 0.001 to about 5% w/v, with 1-2 drops administered 1-4 times per day. For intravitreal, posterior juxtascleral, subtenon, or other type of local delivery, the compounds will generally be in a concentration range of from 0.001% to about 10% w/v.

### EXAMPLES

### Example 1

### Expression and Inhibition Assays of p110α/p85α, p110β/p85α, p110δ/p85α, and p110γ

Class I PI3-Ks can be either purchased (p110α/p85α, p110β/p85α, p110δ/p85α from Upstate, and p110γ from Sigma) or expressed as previously described (Knight et al., 2004). IC50 values are measured using either a standard TLC assay for lipid kinase activity (described below) or a high-throughput membrane capture assay. Kinase reactions are performed by preparing a reaction mixture containing kinase, inhibitor (2% DMSO final concentration), buffer (25 mM HEPES, pH 7.4, 10 mM MgCl2), and freshly sonicated phosphatidylinositol (100 μg/ml). Reactions are initiated by the addition of ATP containing 10 μCi of γ-32P-ATP to a final concentration 10 or 100 μM and allowed to proceed for 5 minutes at room temperature. For TLC analysis, reactions are then terminated by the addition of 105 μl 1N HCl followed by 160 μl CHCl3:MeOH (1:1). The biphasic mixture is vortexed, briefly centrifuged, and the organic phase is transferred to a new tube using a gel loading pipette tip precoated with CHCl₃. This extract is spotted on TLC plates and developed for 3-4 hours in a 65:35 solution of n-propano1:1M acetic acid. The TLC plates are then dried, exposed to a phosphorimager screen (Storm, Amersham), and quantitated. For each compound, kinase activity is measured at 10-12 inhibitor concentrations representing two-fold dilutions from the highest concentration tested (typically, 200 μM). For compounds showing significant activity, IC50 determinations are repeated two to four times, and the reported value is the average of these independent measurements.

Other commercial kits or systems for assaying PI3-K activities are avaiable. The commercially available kits or systems can be used to screen for inhibitors and/or agonists of PI3-Ks including but not limited to PI3-Kinase α, β, δ, and γ. Anr exemplary system is PI3-Kinase (human) HTRF™ Assay from Upstate. The assay can be carried out according to the procedures suggested by the manufacturer. Briefly, the assay is a time resolved FRET assay that indirectly measures PIP3 product formed by the activity of a PI3-K. The kinase reaction is performed in a microtitre plate (e.g., a 384 well microtitre plate). The total reaction volume is approximately 20 ul per well. In the first step, each well receives 2 ul of test compound in 20% dimethylsulphoxide resulting in a 2% DMSO final concentration. Next, approximately 14.5 ul of a kinase/PIP2 mixture (diluted in 1× reaction buffer) is added per well for a final concentration of 0.25-0.3 ug/ml kinase and 10 uM PIP2. The plate is sealed and incubated for 15 minutes at room temperature. To start the reaction, 3.5 ul of ATP (diluted in 1× reaction buffer) is added per well for a final concentration of 10 μM ATP. The plate is sealed and incubated for 1 hour at room temperature. The reaction is stopped by adding 5 ul of Stop Solution per well and then 5 ul of Detection Mix is added per well. The plate is sealed, incubated for 1 hour at room temperature, and then read on an appropriate plate reader. Data is analyzed and IC50s are generated using GraphPad Prism 5.

### Example 2

### Expression and Inhibition Assays of Abl

The compounds described herein can be assayed in triplicate against recombinant full-length Abl or Abl (T315I) (Upstate) in an assay containing 25 mM HEPES, pH 7.4, 10 mM MgCl2, 200 μM ATP (2.5 μCi of γ-32P-ATP), and 0.5 mg/mL BSA. The optimized Abl peptide substrate EAIYAAPFAKKK is used as phosphoacceptor (200 μM). Reactions are terminated by spotting onto phosphocellulose sheets, which are washed with 0.5% phosphoric acid (approximately 6 times, 5-10 minutes each). Sheets are dried and the transferred radioactivity quantitated by phosphorimaging.

### Example 3

### Expression and Inhibition Assays of Hck

The compounds described herein can be assayed in triplicate against recombinant full-length Hck in an assay containing 25 mM HEPES, pH 7.4, 10 mM MgCl2, 200 μM ATP (2.5 μCi of γ-32P-ATP), and 0.5 mg/mL BSA. The optimized Src family kinase peptide substrate EIYGEFKKK is used as phosphoacceptor (200 μM). Reactions are terminated by spotting onto phosphocellulose sheets, which are washed with 0.5% phosphoric acid (approximately 6 times, 5-10 minutes each). Sheets are dried and the transferred radioactivity quantitated by phosphorimaging.

### Example 4

### Expression and Inhibition Assays of Src

The compounds described herein can be assayed in triplicate against recombinant full-length Src or Src (T338I) in an assay containing 25 mM HEPES, pH 7.4, 10 mM MgCl2, 200 μM ATP (2.5 μCi of γ-32P-ATP), and 0.5 mg/mL BSA. The optimized Src family kinase peptide substrate EIYGEFKKK is used as phosphoacceptor (200 μM). Reactions are terminated by spotting onto phosphocellulose sheets, which are washed with 0.5% phosphoric acid (approximately 6 times, 5-10 minutes each). Sheets were dried and the transferred radioactivity quantitated by phosphorimaging.

### Example 5

### Expression and Inhibition Assays of DNA-PK (DNAK)

DNA-PK can be purchased from Promega and assayed using the DNA-PK Assay System (Promega) according to the manufacturer's instructions.

### Example 6

### Expression and Inhibition Assays mTOR

The compounds described herein can be tested against recombinant mTOR (Invitrogen) in an assay containing 50 mM HEPES, pH 7.5, 1mM EGTA, 10 mM MgCl2, 2.5 mM, 0.01% Tween, 10 μM ATP (2.5 μCi of μ-32P-ATP), and 3 μg/mL BSA. Rat recombinant PHAS-1/4EBP1 (Calbiochem; 2 mg/mL) is used as a substrate. Reactions are terminated by spotting onto nitrocellulose, which is washed with 1M NaCl/1% phosphoric acid (approximately 6 times, 5-10 minutes each). Sheets are dried and the transferred radioactivity quantitated by phosphorimaging.

Other kits or systems for assaying mTOR activity are commercially available. For instance, one can use Invitrogen's LanthaScreen™ Kinase assay to test the inhibitors of mTOR disclosed herein. This assay is a time resolved FRET platform that measures the phosphorylation of GFP labeled 4EBP1 by mTOR kinase. The kinase reaction is performed in a white 384 well microtitre plate. The total reaction volume is 20 ul per well and the reaction buffer composition is 50 mM HEPES pH7.5, 0.01% Polysorbate 20, 1 mM EGTA, 10 mM MnCl2, and 2 mM DTT. In the first step, each well receives 2 ul of test compound in 20% dimethylsulphoxide resulting in a 2% DMSO final concentration. Next, 8 ul of mTOR diluted in reaction buffer is added per well for a 60 ng/ml final concentration. To start the reaction, 10 ul of an ATP/GFP-4EBP1 mixture (diluted in reaction buffer) is added per well for a final concentration of 10 uM ATP and 0.5 uM GFP-4EBP1. The plate is sealed and incubated for 1 hour at room temperature. The reaction is stopped by adding 10 ul per well of a Tb-anti-pT46 4EBP1 antibody/EDTA mixture (diluted in TR-FRET buffer) for a final concentration of 1.3 nM antibody and 6.7 mM EDTA. The plate is sealed, incubated for 1 hour at room temperature, and then read on a plate reader set up for LanthaScreen™ TR-FRET. Data is analyzed and IC50s are generated using GraphPad Prism 5.

### Example 7

### In Vivo Effect of mTor Inhibitors on Kinase Substrate Phosphorylation

To explore the tissue specific roles of mTorC1 and mTorC2, mice are treated with an mTORC1/mTORC2 inhibitor and rapamycin, and the acute effect of these drugs on insulin signaling in fat, skeletal muscle and liver tissue are examined.

Approximately 6 week old male C57BL/6 mice are starved of food overnight. Drugs are prepared in about 100 μl of vehicle containing approximately 20% DMSO, 40% PEG-400 and 40% Saline; mTORC1/mTORC2 inhibitor (e.g. 0.4 mg), rapamycin (e.g. 0.1 mg) or vehicle alone is injected IP. After about 30 minutes for the rapamycin-treated mouse or about 10 min for the mTORC1/mTORC2 and vehicle treated mice, approximately 250 mU of insulin in about 100 μl of saline is injected IP. Typically 15 minutes after the insulin injection, the mice are sacrificed by CO2 asphyxiation followed by cervical dislocation. Tissues are harvested and frozen on liquid nitrogen in about 200 μl of cap lysis buffer. The frozen tissue is thawed on ice, manually disrupted with a mortar and pestle, and then further processed with a micro tissue-homogenizer (Fisher PowerGen 125 with Omni-Tip probe). Protein concentration of the cleared lysate is measured by Bradford assay and 5-10 μg of protein is analyzed by western blot.

It is expected that the results will show that in fat and liver, mTORC1/mTORC2 is able to completely inhibit the phosphorylation of Akt at S473 and T308, under the conditions tested, mTORC1/mTORC2 is only partially able to inhibit the phosphorylation of Akt in skeletal muscle. Consistent with this finding, a muscle specific knockout of the integral mTorC2 component rictor also resulted in only a partial loss of Akt phosphorylation at S473.

Rapamycin often stimulates the phosphorylation of Akt, probably by relieving feedback inhibition from S6K to the insulin receptor substrate 1 (IRS1), a key signaling molecule that links activation of the insulin receptor to PI3K activation. In all tissues examined, and especially in fat and muscle, acute rapamycin treatment activates the phosphorylation of Akt at S473 and T308. In contrast to rapamycin, by inhibiting both mTorC2 and mTorC1, mTORC1/mTORC2 inhibitor suppresses rather than enhances Akt activation under the conditions being tested.

Rapamycin and mTORC1/mTORC2 inhibitors differentially affect the mTorC1 substrates S6K and 4EBP1 in vivo. S6 phosphorylation is equally inhibited by rapamycin and mTORC1/mTORC2 inhibitor in all tissues examined. mTORC1/mTORC2 inhibitor is effective at blocking the phosphorylation of 4EBP1 on both T37/46 and S65 in all tissues examined. While rapamycin is more effective at inhibiting the phosphorylation of 4EBP1 in vivo than in cell culture experiments, rapamycin never blocks 4EBP1 phosphorylation as completely as mTORC1/mTORC2 inhibitor.

Rapamycin has been a powerful pharmacological tool allowing the discovery of mTor's central role in the control of protein synthesis. Since the discovery of a rapamycin-insensitive mTor complex there has been a significant effort to develop pharmacological tools for studying this complex. Here mTORC1/mTORC2 inhibitors used to chemically dissect the effects of mTor kinase inhibition toward mTorC1 and mTorC2 activity. The results of the present invention are expected to shown through the use of these inhibitors that the inhibition of mTor kinase activity is sufficient to prevent the phosphorylation of Akt at S473, under the conditions tested, providing further evidence that mTorC2 may be the kinase responsible for Akt hydrophobic motif phosphorylation. The results of the present invention further are expected to provide that phosphorylation at T308 is probably linked to phosphorylation at S473, as had been observed in experiments where mTorC2 was disabled by RNAi, but not homologous recombination. However, inhibition of mTorC2 does not result in a complete block of Akt signaling, as T308P is partially maintained and Akt substrate phosphorylation is only modestly affected when S473 is not phosphorylated under the conditions tested. Despite its modest effect on Akt substrates, the mTORC1/mTORC2 inhibitor is a more effective anti-proliferative agent than rapamycin. These results are reproduced even in cells lacking mTorC2 (SIN1−/−), suggesting that downstream mTorC1 substrates might be responsible for the mTORC1/mTORC2 inhibitor's strong anti-proliferative effects. The results of the present invention are expected to provide that phosphorylation of the mTorC1 substrate 4EBP1 is partially resistant to rapamycin treatment at concentrations that fully inhibit p70S6K while mTORC1/mTORC2 inhibitor completely inhibits both p70S6K and 4EBP1. Consequently, the enhanced block of cell proliferation by the mTORC1/mTORC2 inhibitor compared with rapamycin may reflect in part its ability to more efficiently inhibit eIF4E-dependent translation control.

### Example 8

### Expression and Inhibition Assays of Vascular Endothelial Growth Receptor

The compounds described herein can be tested against recombinant KDR receptor kinase domain (Invitrogen) in an assay containing 25 mM HEPES, pH 7.4, 10 mM MgCl2, 0.1% BME, 10 μM ATP (2.5 μCi of μ-32P-ATP), and 3 μg/mL BSA. Poly E-Y (Sigma; 2 mg/mL) is used as a substrate. Reactions are terminated by spotting onto nitrocellulose, which is washed with 1M NaCl/1% phosphoric acid (approximately 6 times, 5-10 minutes each). Sheets are dried and the transferred radioactivity quantitated by phosphorimaging.

### Example 9

### Expression and Inhibition Assays of Ephrin Receptor B4 (EphB4)

The compounds described herein can be tested against recombinant Ephrin receptor B4 kinase domain (Invitrogen) in an assay containing 25 mM HEPES, pH 7.4, 10 mM MgCl2, 0.1% BME, 10 μM ATP (2.5 μCi of μ-32P-ATP), and 3 μg/mL BSA. Poly E-Y (Sigma; 2 mg/mL) is used as a substrate. Reactions are terminated by spotting onto nitrocellulose, which is washed with 1M NaCl/1% phosphoric acid (approximately 6 times, 5-10 minutes each). Sheets are dried and the transferred radioactivity quantitated by phosphorimaging.

### Example 10

### Expression and Inhibition Assays of Epidermal Growth Factor Receptor (EGFR)

The compounds described herein can be tested against recombinant EGF receptor kinase domain (Invitrogen) in an assay containing 25 mM HEPES, pH 7.4, 10 mM MgCl2, 0.1% BME, 10 μM ATP (2.5 μCi of μ-32P-ATP), and 3 μg/mL BSA. Poly E-Y (Sigma; 2 mg/mL) is used as a substrate. Reactions are terminated by spotting onto nitrocellulose, which is washed with 1M NaCl/1% phosphoric acid (approximately 6 times, 5-10 minutes each). Sheets are dried and the transferred radioactivity quantitated by phosphorimaging.

### Example 11

### Expression and Inhibition Assays of KIT

The compounds described herein can be tested against recombinant KIT kinase domain (Invitrogen) in an assay containing 25 mM HEPES, pH 7.4, 10 mM MgCl2, 1 mM DTT, 10 mM MnCl2, 10 μM ATP (2.5 μCi of μ-32P-ATP), and 3 μg/mL BSA. Poly E-Y (Sigma; 2 mg/mL) is used as a substrate. Reactions are terminated by spotting onto nitrocellulose, which is washed with 1M NaCl/1% phosphoric acid (approximately 6 times, 5-10 minutes each). Sheets are dried and the transferred radioactivity quantitated by phosphorimaging.

### Example 12

### Expression and Inhibition Assays of RET

The compounds described herein can be tested against recombinant RET kinase domain (Invitrogen) in an assay containing 25 mM HEPES, pH 7.4, 10 mM MgCl2, 2.5 mM DTT, 10 μM ATP (2.5 μCi of μ-32P-ATP), and 3 μg/mL BSA. The optimized Abl peptide substrate EAIYAAPFAKKK is used as phosphoacceptor (200 μM). Reactions are terminated by spotting onto phosphocellulose sheets, which are washed with 0.5% phosphoric acid (approximately 6 times, 5-10 minutes each). Sheets are dried and the transferred radioactivity quantitated by phosphorimaging.

### Example 13

### Expression and Inhibition Assays of Platelet Derived Growth Factor Receptor (PDGFR)

The compounds described herein can be tested against recombinant PDG receptor kinase domain (Invitrogen) in an assay containing 25 mM HEPES, pH 7.4, 10 mM MgCl2, 2.5 mM DTT, 10 μM ATP (2.5 μCi of μ-32P-ATP), and 3 μg/mL BSA. The optimized Abl peptide substrate EAIYAAPFAKKK is used as phosphoacceptor (200 μM). Reactions are terminated by spotting onto phosphocellulose sheets, which are washed with 0.5% phosphoric acid (approximately 6 times, 5-10 minutes each). Sheets are dried and the transferred radioactivity quantitated by phosphorimaging.

### Example 14

### Expression and Inhibition Assays of FMS-Related Tyrosine Kinase 3 (FLT-3)

The compounds described herein can be tested against recombinant FLT-3 kinase domain (Invitrogen) in an assay containing 25 mM HEPES, pH 7.4, 10 mM MgCl2, 2.5 mM DTT, 10 μM ATP (2.5 μCi of μ-32P-ATP), and 3 μg/mL BSA. The optimized Abl peptide substrate EAIYAAPFAKKK is used as phosphoacceptor (200 μM). Reactions are terminated by spotting onto phosphocellulose sheets, which are washed with 0.5% phosphoric acid (approximately 6 times, 5-10 minutes each). Sheets are dried and the transferred radioactivity quantitated by phosphorimaging.

### Example 15

### Expression and Inhibition Assays of TEK Receptor Tyrosine Kinase (TIE2)

The compounds described herein can be tested against recombinant TIE2 kinase domain (Invitrogen) in an assay containing 25 mM HEPES, pH 7.4, 10 mM MgCl2, 2 mM DTT, 10 mM MnCl2, 10 μM ATP (2.5 μCi of μ-32P-ATP), and 3 μg/mL BSA. Poly E-Y (Sigma; 2 mg/mL) is used as a substrate. Reactions are terminated by spotting onto nitrocellulose, which is washed with 1M NaCl/1% phosphoric acid (approximately 6 times, 5-10 minutes each). Sheets are dried and the transferred radioactivity quantitated by phosphorimaging.

### Example 16

### Akt Kinase Assay

Cells comprising components of the Akt/mTOR pathway, including but not limited to L6 myoblasts, B-ALL cells, B-cells, T-cells, leukemia cells, bone marrow cells, p190 transduced cells, philladelphia chromosome positive cells (Ph+), and mouse embryonic fibroblasts, are typically grown in cell growth media such as DMEM supplemented with fetal bovine serum and/or antibiotics, and grown to confluency.

In order to compare the effect of one or more compounds disclosed herein on Akt activation, said cells are serum starved overnight and incubated with one or more compounds disclosed herein or about 0.1% DMSO for approximately 1 minute to about 1 hour prior to stimulation with insulin (e.g. 100 nM) for about 1 minutes to about 1 hour. Cells are lysed by scraping into ice cold lysis buffer containing detergents such as sodium dodecyl sulfate and protease inhibitors (e.g., PMSF). After contacting cells with lysis buffer, the solution is briefly sonicated, cleared by centrifugation, resolved by SDS-PAGE, transferred to nitrocellulose or PVDF and immunoblotted using antibodies to phospho-Akt 5473, phospho-Akt T308, Akt, and (3-actin (Cell Signaling Technologies).

It is expected that one or more compounds of the present disclosure inhibit insulin stimulated phosphorylation of Akt at S473. Alternatively, some compounds disclosed herein additionally inhibit insulin stimulated phosphorylation of Akt at T308. Such class of compounds can inhibit Akt more effectively than rapamycin and may be indicative of mTORC2 inhibitors or inhibitors of upstream kinases such as PI3K or Akt.

### Example 17

### B Cell Activation and Proliferation Assay

The ability of one or more subject compounds to inhibit B cell activitation and proliferation is determined according to standard procedures known in the art. For example, an in vitro cellular proliferation assay is established that measures the metabolic activity of live cells. The assay is performed in a 96 well microtiter plate using Alamar Blue reduction. Balb/c splenic B cells are purified over a Ficoll-Paque™ PLUS gradient followed by magnetic cell separation using a MACS B cell Isolation Kit (Miletenyi). Cells are plated in 90 ul at 50,000 cells/well in B Cell Media (RPMI+10% FBS+Penn/Strep+50 uM bME+5 mM HEPES). A compound disclosed herein is diluted in B Cell Media and added in a 10 ul volume. Plates are incubated for 30 min at 37 C and 5% CO₂ (0.2% DMSO final concentration). A 50 ul B cell stimulation cocktail is then added containing either 10 ug/ml LPS or 5 ug/ml F(ab′)2 Donkey anti-mouse IgM plus 2 ng/ml recombinant mouse IL4 in B Cell Media. Plates are incubated for 72 hours at 37° C. and 5% CO₂. A volume of 15 uL of Alamar Blue reagent is added to each well and plates are incubated for 5 hours at 37 C and 5% CO₂. Alamar Blue fluoresce is read at 560 Ex/590 Em, and IC50 values are calculated using GraphPad Prism 5.

### Example 18

### Cell Culture of Epithelial Cells of Ocular Origin

Ocular epithelial cells are obtained within 5 days postmortem post-mortem from corneas preserved under cold storage conditions in Optisol (Bausch and Lomb, Irvine, Calif.) or from corneal biopsy from living donors. The tissue is washed with phosphate-buffered saline and incubated in Dispase II (Roche Diagnostics, Basel, Switzerland) at 37° C. for 30 minutes, and the epithelial surface is gently scraped to separate the epithelium from the underlying stroma. The separated epithelium is then incubated and pipetted in trypsin-ethylenediaminetetraacetic acid to obtain a single cell suspension. The trypsin is then neutralized with corneal epithelium culture medium. Corneal epithelium culture medium is composed of Dulbecco modified Eagle medium:F12 basal media in a 2:1 ratio containing 10% irradiated fetal bovine serum, hydrocortisone 0.4 μg/mL, cholera toxin 0.1 nmol, recombinant human insulin 5 μg/mL, and epidermal growth factor 10 ng/mL, and the antimicrobials penicillin (100 IU/mL), streptomycin (100 μg/mL), and amphotericin B (0.25 μg/mL). Cells are mainlined by sub-culturing at a 1:4 ratio after reaching 80% confluency. Ocular epithelial cells are screened for inhibition of proliferation or toxicity by contacting a test compound with the cells and assaying for viability using the commercially available MTT assay (Promega).

### Example 19

### Cell Culture of Endothelial Cells of Ocular Origin

All tissues are maintained at 4° C. in storage medium (Optisol; Chiron Vision, Irvine, Calif.) for less than 10 days before study. The tissue is rinsed three times with DMEM containing 50 mg/mL gentamicin and 1.25 mg/mL amphotericin B. The central cornea is removed by a trephine of 8-mm diameter. Afterward, the Descemet's membrane and corneal endothelial cells are stripped from the posterior surface of the peripheral corneoscleral tissue under a dissecting microscope and digested at 37° C. for 1.5 to 16 hours with 2 mg/mL collagenase A in supplemented hormonal epithelial medium (SHEM), which is made of an equal volume of HEPES-buffered DMEM and Ham's F12 supplemented with 5% FBS, 0.5% dimethyl sulfoxide, 2 ng/mL mouse EGF, 5 μg/mL insulin, 5 μg/mL transferrin, 5 ng/mL selenium, 0.5 μg/mL hydrocortisone, 1 nM cholera toxin, 50 μg/mL gentamicin, and 1.25 μg/mL amphotericin B. After digestion, HCECs formed aggregates, which are collected by centrifugation at 2000 rpm for 3 minutes to remove the digestion solution. As a control, Descemet's membrane strips are also digested in 10 mg/mL Dispase II in SHEM and trypsin/EDTA for up to 3 hours.

### Preservation of Isolated HCEC Aggregates

The resultant aggregates of HCECs are preserved in KSFM with complete supplement (storage medium 1), DMEM/F12 with KSFM supplements (storage medium 2), or DMEM/F12 with SHEM supplements without FBS (storage medium 3). All these media are serum free, one of the major differences among them is the calcium concentration, which is 0.09 mM in storage medium 1, but is 1.05 mM in storage media 2 and 3. HCEC aggregates are stored in a tissue culture incubator at 37° C. for up to 3 weeks. Cell viability is determined (Live and Dead assay; Invitrogen) and also evaluated by subculturing them in SHEM.

### Expansion of Isolated HCEC Aggregates

The resultant HCEC aggregates, either immediately after digestion or after a period of preservation in a storage medium, are then cultured in SHEM with or without additional growth factors such as 40 ng/mL bFGF, 0.1 mg/mL BPE, and 20 ng/mL NGF on a plastic dish under 37° C. and 5% CO2. The media are changed every 2 to 3 days. Some HCEC aggregates are pretreated with trypsin/EDTA at 37° C. for 10 minutes to dissociate endothelial cells before the aforementioned cultivation.

### Immunostaining

HCEC aggregates are embedded in OCT and subjected to frozen sectioning. Cryosections of 4 μm are air-dried at room temperature (RT) for 30 minutes, and fixed in cold acetone for 10 minutes at −20° C. Sections used for immunostaining are rehydrated in PBS, and incubated in 0.2% Triton X-100 for 10 minutes. After three rinses with PBS for 5 minutes each and preincubation with 2% BSA to block nonspecific staining, the sections are incubated with anti-laminin 5, type IV collagen, perlecan, ZO-1, and connexin 43 (all at 1:100) antibodies for 1 hour. After three washes with PBS for 15 minutes, the sections are incubated with a FITC-conjugated secondary antibody (goat anti-rabbit or anti-mouse IgG at 1:100) for 45 minutes. After three additional PBS washes, each for 10 minutes, they are counterstained with propidium iodide (1:1000) or Hoechst 33342 (10 μg/mL), then mounted with an antifade solution and analyzed with a fluorescence microscope. HCECs cultured in 24-well plates or chamber slides are fixed in 4% paraformaldehyde for 15 minutes at RT and stained with anti-ZO-1 and connexin 43 antibodies as just described. For immunohistochemical staining of Ki67, endogenous peroxidase activity is blocked by 0.6% hydrogen peroxide for 10 minutes. Nonspecific staining is blocked by 1% normal goat serum for 30 minutes. Cells are then incubated with anti-Ki67 antibody (1:100) for 1 hour. After three washes with PBS for 15 minutes, cells are incubated with biotinylated rabbit anti-mouse IgG (1:100) for 30 minutes, followed by incubation with ABC reagent for 30 minutes. The reaction product is developed with DAB for 5 minutes and examined by light microscope.

### Cell-Viability and TUNEL Assays

Cell-viability and terminal deoxyribonucleotidyl transferase-mediated FITC-linked dUTP nick-end DNA labeling (TUNEL) assays are used to determine living and apoptotic cells, respectively. HCEC aggregates are incubated with cell-viability assay reagents for 15 minutes at RT. Live cells are distinguished by green fluorescence staining of the cell cytoplasm, and dead cells are stained with red fluorescence in the nuclei. The TUNEL assay is performed according to the manufacturer's instructions. Briefly, cross-sections of HCEC aggregates are fixed in 4% paraformaldehyde for 20 minutes at RT and permeabilized with 1% Triton X-100. Samples are then incubated for 60 minutes at 37° C. with exogenous TdT and fluorescein-conjugated dUTP, for repair of nicked 3′-hydroxyl DNA ends. Cells are treated with DNase I as the positive control, whereas negative control cells are incubated with a buffer lacking the rTdT enzyme. The apoptotic nuclei are labeled with green fluorescence.

### Example 20

### Cell Culture of Retinal Cells

Eyes are cut in half along their equator and the neural retina is dissected from the anterior part of the eye in buffered saline solution, according to standard methods known in the art. Briefly, the retina, ciliary body, and vitreous are dissected away from the anterior half of the eye in one piece, and the retina is gently detached from the clear vitreous. Each retina is dissociated with papain (Worthington Biochemical Corporation, Lakewood, N.J.), followed by inactivation with fetal bovine serum (FBS) and addition of 134 Kunitz units/ml of DNaseI. The enzymatically dissociated cells are triturated and collected by centrifugation, resuspended in Dulbecco's modified Eagle's medium (DMEM)/F12 medium (Gibco BRL, Invitrogen Life Technologies, Carlsbad, Calif.) containing 25 μg/ml of insulin, 100 μg/ml of transferrin, 60 μM putrescine, 30 nM seleni 20 nM progesterone, 100 U/ml of penicillin, 100 μg/ml of streptomycin, 0.05 M Hepes, and 10% FBS. Dissociated primary retinal cells are plated onto Poly-D-lysine- and Matrigel- (BD, Franklin Lakes, N.J.) coated glass coverslips that are placed in 24-well tissue culture plates (Falcon Tissue Culture Plates, Fisher Scientific, Pittsburgh, Pa.). Cells are maintained in culture for 5 days to one month in 0.5 ml of media (as above, except with only 1% FBS) at 37° C. and 5% CO2.

### Immunocytochemistry Analysis

The retina 1 neuronal cells are cultured for 1, 3, 6, and 8 weeks in the presence and absence of test compounds of the present invention, and the cells are analyzed by immunohistochemistry at each time point. Immunocytochemistry analysis is performed according to standard techniques known in the art. Rod photoreceptors are identified by labeling with a rhodopsin-specific antibody (mouse monoclonal, diluted 1:500; Chemicon, Temecula, Calif.). An antibody to mid-weight neurofilament (NFM rabbit polyclonal, diluted 1:10,000, Chemicon) is used to identify ganglion cells; an antibody to β3-tubulin (G7121 mouse monoclonal, diluted 1:1000, Promega, Madison, Wis.) is used to generally identify interneurons and ganglion cells, and antibodies to calbindin (AB1778 rabbit polyclonal, diluted 1:250, Chemicon) and calretinin (AB5054 rabbit polyclonal, diluted 1:5000, Chemicon) are used to identify subpopulations of calbindin- and calretinin-expressing interneurons in the inner nuclear layer. Briefly, the retinal cell cultures are fixed with 4% paraformaldehyde (Polysciences, Inc, Warrington, Pa.) and/or ethanol, rinsed in Dulbecco's phosphate buffered saline (DPBS), and incubated with primary antibody for 1 hour at 37° C. The cells are then rinsed with DPBS, incubated with a secondary antibody (Alexa 488- or Alexa 568-conjugated secondary antibodies (Molecular Probes, Eugene, Oreg.)), and rinsed with DPBS. Nuclei are stained with 4′,6-diamidino-2-phenylindole (DAPI, Molecular Probes), and the cultures are rinsed with DPBS before removing the glass coverslips and mounting them with Fluoromount-G (Southern Biotech, Birmingham, Ala.) on glass slides for viewing and analysis.

### Example 21

### Matrigel Plug Angiogenesis Assay

Matrigel containing test compounds are injected subcutaneously or intraocularly, where it solidifies to form a plug. The plug is recovered after 7-21 days in the animal and examined histologically to determine the extent to which blood vessels have entered it. Angiogenesis is measured by quantification of the vessels in histologic sections. Alternatively, fluorescence measurement of plasma volume is performed using fluorescein isothiocyanate (FITC)-labeled dextran 150. The results are expected to indicate one or more compounds disclosed herein that inhibit angiogenesis and are thus expected to be useful in treating ocular disorders related to aberrant angiogenesis and/or vascular permeability.

### Example 22

### The Corneal Angiogenesis Assay

A pocket is made in the cornea, and a plug containing an angiogenesis inducing formulation (e.g. VEGF, FGF, or tumor cells), when introduced into this pocket, elicits the ingrowth of new vessels from the peripheral limbal vasculature. Slow-release materials such as ELVAX (ethylene vinyl copolymer) or Hydron are used to introduce angiogenesis inducing substances into the corneal pocket. Alternatively, a sponge material is used.

The effect of putative inhibitors on the locally induced (e.g., sponge implant) angiogenic reaction in the cornea (e.g., by FGF, VEGF, or tumor cells). The test compound is administered orally, systemically, or directly to the eye. Systemic administration is by bolus injection or, more effectively, by use of a sustained-release method such as implantation of osmotic pumps loaded with the test inhibitor. Administration to the eye is by any of the methods described herein including but not limited to eye drops, topical administration of a cream, emulsion, or gel, intravitreal injection.

The vascular response is monitored by direct observation throughout the course of the experiment using a stereomicroscope in mice. Definitive visualization of the corneal vasculature is achieved by administration of fluorochrome-labeled high-molecular weight dextran. Quantification is performed by measuring the area of vessel penetration, the progress of vessels toward the angiogenic stimulus over time, or in the case of fluorescence, histogram analysis or pixel counts above a specific (background) threshold.

The results are expected to indicate one or more compounds disclosed herein that inhibit angiogenesis and are thus expected to be useful in treating ocular disorders related to aberrant angiogenesis and/or vascular permeability.

### Example 23

### Microtiter-Plate Angiogenesis Assay

The assay plate is prepared by placing a collagen plug in the bottom of each well with 5-10 cell spheroids per collagen plug each spheroid containing 400-500 cells. Each collagen plug is covered with 1100 μl of storage medium per well and stored for future use (1-3 days at 37° C., 5% CO₂). The plate is sealed with sealing. Test compounds are dissolved in 200₁11 assay medium with at least one well including a VEGF positive control and at least one well without VEGF or test compound as a negative control. The assay plate is removed from the incubator and storage medium is carefully pipeted away. Assay medium containing the test compounds are pipeted onto the collagen plug. The plug is placed in a humidified incubator for (37° C., 5% CO₂) 24-48 hours. Angiogenesis is quantified by counting the number of sprouts, measuring average sprout length, or determining cumulative sprout length. The assay can be preserved for later analysis by removing the assay medium, adding 1 ml of 10% paraformaldehyde in Hanks BSS per well, and storing at 4° C. The results are expected to identify compounds that inhibit angiogenesis in various cell types tested, including cells of ocular origin.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of modulating vascular endothelial growth factor (VEGF) signaling pathway in a cell of ocular origin, comprising administering a phosphoinositide 3-kinase (PI3K) antagonist or pharmaceutically acceptable salt thereof into said cell, wherein said antagonist inhibits one or more of PI3Kα, PI3KIβ, PI3Kγ, or PI3Kδ with an IC50 of less than about 1 μM.

2. A method of mitigating an ophthalmic disease comprising administering an mTOR antagonist or a pharmaceutically acceptable salt thereof to a subject's eye, wherein said antagonist inhibits both mTORC1 and mTORC2 with an IC50 less than about 1 μM.

3. A method of inhibiting angiogenesis in a subject's eye comprising administering a combination of an mTOR antagonist and a PI3K antagonist or pharmaceutically acceptable salts thereof to the eye, wherein said mTOR antagonist inhibits both mTORC1 and mTORC2 with an IC50 less than 1 μM, and wherein said PI3K antagonist inhibits one or more of PI3Kα, PI3KIβ, PI3Kγ, or PI3Kδ with an IC50 of less than 1 μM.

4. The method of claim 1 or 3, wherein the PI3K antagonist inhibits PI3Kδ and PI3Kγ selectively with an IC50 value of less than 100 nM.

5. The method of any one of the preceding claims, wherein said administering comprises the use of an eye drop.

6. The method of any one of the preceding claims, wherein said administering comprises intravitreal injection.

7. The method of any one of the preceding claims, wherein said administering comprises the use of an excipient formulation that increases intraocular penetration of said antagonist.

8. A method of treating an ophthalmic disease comprising administering to a subject in need thereof an agent that inhibits mTORC1 and a PI3K antagonist.

9. The method of claim 8 wherein the agent that inhibits mTORC 1 is rapamycin or a rapalogue, and the PI3K antagonist selectively inhibits PI3Kγ and/or PI3Kδ with an IC50 of less than 1 μM.

10. A drug eluting device for treatment of an ophthalmic disease, wherein said device releases one or more pyrazolopyrimidine kinase inhibitors or pharmaceutically acceptable salts thereof to the eye of a subject over a period of time.

11. The device of claim 10, wherein said device comprises a polymer.

12. The device of claim 11, wherein said polymer comprises a biodegradable polymer selected from the group consisting of polyglycolide, polylactide, poly ε-caprolactone, polyglyconate, polyhydroxybutyrate, polyhydroxyvalerate, and polydioxanone.

13. The device of claim 11, wherein said polymer comprises a non-biodegradable polymer selected from the group consisting of silicone, acrylates, polyethylenes, polyurethane, polyester, polypropylene, polytetrafluoroethylene, poly ether ketone, nylon, collagen, polyethylene terepthalate, polycarbonate, and polyimide.

14. The device of claim 10, wherein the pyrazolopyrimidine kinase inhibitor has a structure of Formula I: 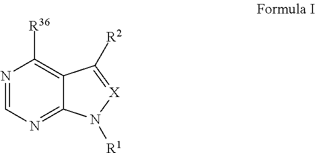
wherein
X is ═N— or ═C(H)—;
R¹ is hydrogen, R³-substituted or unsubstituted alkyl, R³-substituted or unsubstituted heteroalkyl, R³-substituted or unsubstituted cycloalkyl, R³-substituted or unsubstituted heterocycloalkyl, R³-substituted or unsubstituted aryl, or R³-substituted or unsubstituted heteroaryl;
R² is halogen, R⁴-substituted aryl, or substituted or unsubstituted heteroaryl;
R³ is halogen, —CN, —OR⁵, —S(O)ₙR⁶, —NR⁷R⁸, —C(O)R⁹, ═N—NH₂, —NR¹⁰—C(O)R¹¹, —NR¹²—C(O)—OR¹³, —C(O)NR¹⁴R¹⁵, —NR¹⁶S(O)₂R¹⁷, —S(O)₂NR¹⁸, R¹⁹-substituted or unsubstituted alkyl, R¹⁹-substituted or unsubstituted heteroalkyl, R¹⁹-substituted or unsubstituted cycloalkyl, R¹⁹-substituted or unsubstituted heterocycloalkyl, R¹⁹-substituted or unsubstituted aryl, or R¹⁹-substituted or unsubstituted heteroaryl, wherein n is an integer from 0 to 2;
R³⁶ is —NR³⁷R³⁸ or halogen;
R⁴ is halogen, —CN, —OR²⁰, —S(O)_{q}R²¹, —NR²²R²³, —C(O)R²⁴, ═N—NH₂, —NR²⁵—C(O)R²⁶, —NR²⁷—C(O)—OR²⁸, —C(O)NR²⁹R³⁰, —NR³¹S(O)₂R³², —S(O)₂NR³³, R³⁴-substituted or unsubstituted alkyl, R³⁴-substituted or unsubstituted heteroalkyl, R³⁴-substituted or unsubstituted cycloalkyl, R³⁴-substituted or unsubstituted heterocycloalkyl, R³⁴-substituted or unsubstituted aryl, or R³⁴-substituted or unsubstituted heteroaryl, wherein q is an integer from 0 to 2;
R⁵, R⁶, R⁷, R⁸, R⁹, R¹⁰, R¹¹, R¹², R¹³, R¹⁴, R¹⁵, R¹⁶, R¹⁷, R¹⁸, R²⁰, R²¹, R²², R²³, R²⁴, R²⁵, R²⁶, R²⁷, R²⁸, R²⁹, R³⁰, R³¹, R³², and R³³ are independently hydrogen, R³⁵-substituted or unsubstituted alkyl, R³⁵-substituted or unsubstituted heteroalkyl, unsubstituted cycloalkyl, R³⁵-substituted or unsubstituted heterocycloalkyl, R³⁵-substituted or unsubstituted aryl, or R³⁵-substituted or unsubstituted heteroaryl;
R¹⁹, R³⁴ and R³⁵ are independently hydrogen, halogen, unsubstituted alkyl, unsubstituted heteroalkyl, unsubstituted cycloalkyl, unsubstituted heterocycloalkyl, unsubstituted aryl, or unsubstituted heteroaryl; and
R³⁷ and R³⁸ are hydrogen, halogen, or unsubstituted alkyl.

15. The method of claims 1-7, wherein the antagonist has a structure of Formula I: 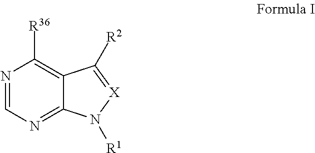
wherein
X is ═N— or ═C(H)—;
R¹ is hydrogen, R³-substituted or unsubstituted alkyl, R³-substituted or unsubstituted heteroalkyl, R³-substituted or unsubstituted cycloalkyl, R³-substituted or unsubstituted heterocycloalkyl, R³-substituted or unsubstituted aryl, or R³-substituted or unsubstituted heteroaryl;
R² is halogen, R⁴-substituted aryl, or substituted or unsubstituted heteroaryl;
R³ is halogen, —CN, —OR⁵, —S(O)ₙR⁶, —NR⁷R⁸, —C(O)R⁹, ═N—NH₂, —NR¹⁰—C(O)R¹¹, —NR¹²—C(O)—OR¹³, —C(O)NR¹⁴R¹⁵, —NR¹⁶S(O)₂R¹⁷, —S(O)₂NR¹⁸, R¹⁹-substituted or unsubstituted alkyl, R¹⁹-substituted or unsubstituted heteroalkyl, R¹⁹-substituted or unsubstituted cycloalkyl, R¹⁹-substituted or unsubstituted heterocycloalkyl, R¹⁹-substituted or unsubstituted aryl, or R¹⁹-substituted or unsubstituted heteroaryl, wherein n is an integer from 0 to 2;
R³⁶ is —NR³⁷R³⁸ or halogen;
R⁴ is halogen, —CN, —OR²⁰, —S(O)_{q}R²¹, —NR²²R²³, —C(O)R²⁴, ═N—NH₂, —NR²⁵—C(O)R²⁶, —NR²⁷—C(O)—OR²⁸, —C(O)NR²⁹R³⁰, —NR³¹S(O)₂R³², —S(O)₂NR³³, R³⁴-substituted or unsubstituted alkyl, R³⁴-substituted or unsubstituted heteroalkyl, R³⁴-substituted or unsubstituted cycloalkyl, R³⁴-substituted or unsubstituted heterocycloalkyl, R³⁴-substituted or unsubstituted aryl, or R³⁴-substituted or unsubstituted heteroaryl, wherein q is an integer from 0 to 2;
R⁵, R⁶, R⁷, R⁸, R⁹, R¹⁰, R¹¹, R¹², R¹³, R¹⁴, R¹⁵, R¹⁶, R¹⁷, R¹⁸, R²⁰, R²¹, R²², R²³, R²⁴, R²⁴, R²⁵, R²⁶, R²⁷, R²⁸, R²⁹, R³⁰, R³¹, R³², and R³³ are independently hydrogen, R³⁵-substituted or unsubstituted alkyl, R³⁵-substituted or unsubstituted heteroalkyl, unsubstituted cycloalkyl, R³⁵-substituted or unsubstituted heterocycloalkyl, R³⁵-substituted or unsubstituted aryl, or R³⁵-substituted or unsubstituted heteroaryl;
R¹⁹, R³⁴ and R³⁵ are independently hydrogen, halogen, unsubstituted alkyl, unsubstituted heteroalkyl, unsubstituted cycloalkyl, unsubstituted heterocycloalkyl, unsubstituted aryl, or unsubstituted heteroaryl; and R³⁷and R³⁸ are hydrogen, halogen, or unsubstituted alkyl.

16. The method of claims 1-7, wherein the antagonist has a structure of Formula XV 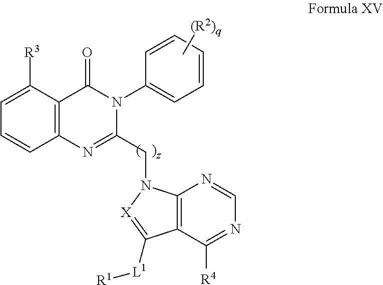
wherein
q is an integer from 0 to 5;
z is an integer from 0 to 10;
X is ═CH— or ═N—;
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ and R² are independently halogen, —CN, —OR⁵, —S(O)ₙR⁶, —NR⁷R⁸, —C(O)R⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein n is independently an integer from 0 to 2;
R³, and R⁴ are independently hydrogen, halogen, —CN, —OR⁵, —S(O)ₙR⁶, —NR⁷R⁸, —C(O)R⁹, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is independently hydrogen, —C(O)R¹⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ is independently hydrogen, —NR¹¹R¹², substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein if n is 1 or 2 then R⁶ is other than hydrogen;
R⁷ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is independently hydrogen, —S(O)ₙR¹³, —C(O)R¹⁴, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁹ is independently —NR¹⁵R¹⁶, hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁰ is independently hydrogen, —NR¹⁷R¹⁸, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁴ is independently hydrogen, —NR¹⁹R²⁰, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
R¹¹, R¹², R¹³, R¹⁵, R¹⁶, R¹⁷, R¹⁸, R¹⁹, and R²⁰ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.


SEQUENCE LISTING (2 sequences)

SEQ ID NO: 1 - protein, 12 aa, Artificial Sequence
  Description of Artificial Sequence Synthetic peptide
EAIYAAPFAKKK

SEQ ID NO: 2 - protein, 9 aa, Artificial Sequence
  Description of Artificial Sequence Synthetic peptide
EIYGEFKKK